SEQUENCE LISTING (113 sequences)

SEQ ID NO: 1 - protein, 832 aa, Thermus aquaticus [sequence omitted]

SEQ ID NO: 2 - DNA, 32 bp, artificial
  oligonucleotide probes or primers or compounds
gcgtaactagaccataagccgatagcttgaac

SEQ ID NO: 3 - DNA, 32 bp, artificial; identical to SEQ ID NO: 2
  oligonucleotide probes or primers or compounds

SEQ ID NO: 4 - DNA, 67 bp, artificial
  oligonucleotide probes or primers or compounds
cggaccagcaagcttagagatcacggtatccagattcgctcatagtacacaactgcctccggttcaa

SEQ ID NO: 5 - DNA, 21 bp, artificial
  oligonucleotide probes or primers or compounds
gcagttgtgtactatgagcga

SEQ ID NO: 6 - DNA, 20 bp, artificial
  oligonucleotide probes or primers or compounds
gcgtaactagaccataagcc

SEQ ID NO: 7 - DNA, 18 bp, artificial
  oligonucleotide probes or primers or compounds
ctacctgagtggcatcct

SEQ ID NO: 8 - DNA, 18 bp, artificial
  oligonucleotide probes or primers or compounds
ctacctgagtggcatcct

SEQ ID NO: 9 - DNA, 18 bp, artificial
  oligonucleotide probes or primers or compounds
cagaaaatacctacctga

SEQ ID NO: 10 - DNA, 18 bp, artificial
  oligonucleotide probes or primers or compounds
gcttttaaccagagtggc

SEQ ID NO: 11 - DNA, 32 bp, artificial; identical to SEQ ID NO: 2
  oligonucleotide probes or primers or compounds

SEQ ID NO: 12 - DNA, 79 bp, artificial
  oligonucleotide probes or primers or compounds
  misc_feature (17)..(20): n is a, c, g, or t
  misc_feature (75)..(79): n is a, c, g, or t
ccgcaattggcgtgatnnnnagatcggaagagcgtcgtgtagtccgcatgtcgcgtgatagggatatcttgcggnnnnn

SEQ ID NO: 13 - DNA, 79 bp, artificial
  oligonucleotide probes or primers or compounds
  misc_feature (17)..(20): n is a, c, g, or t
  misc_feature (75)..(79): n is a, c, g, or t
ccgcaattggacatcgnnnnagatcggaagagcgtcgtgtagtccgcatgtcgcgtgatagggatatcttgcggnnnnn

SEQ ID NO: 14 - DNA, 79 bp, artificial
  oligonucleotide probes or primers or compounds
  misc_feature (17)..(20): n is a, c, g, or t
  misc_feature (75)..(79): n is a, c, g, or t
ccgcaattggcactgtnnnnagatcggaagagcgtcgtgtagtccgcatgtcgcgtgatagggatatcttgcggnnnnn

SEQ ID NO: 15 - DNA, 79 bp, artificial
  oligonucleotide probes or primers or compounds
  misc_feature (17)..(20): n is a, c, g, or t
  misc_feature (75)..(79): n is a, c, g, or t
ccgcaattggattggcnnnnagatcggaagagcgtcgtgtagtccgcatgtcgcgtgatagggatatcttgcggnnnnn

SEQ ID NO: 16 - DNA, 18 bp, artificial
  oligonucleotide probes or primers or compounds
ctatcacgcgacatgcgg

SEQ ID NO: 17 - DNA, 52 bp, artificial
  oligonucleotide probes or primers or compounds
  misc_feature (47)..(52): n is a, c, g, or t
ccgcaagatcggaagagcggttcagcaggaatgcatatgcttgcggnnnnnn

SEQ ID NO: 18 - DNA, 58 bp, artificial
  oligonucleotide probes or primers or compounds
aatgatacggcgaccaccgagatctacactctttccctacacgacgctcttccgatct

SEQ ID NO: 19 - DNA, 62 bp, artificial
  oligonucleotide probes or primers or compounds
caagcagaagacggcatacgagatcggtctcggcattcctgctgaaccgctcttccgatctt

SEQ ID NO: 20 - DNA, 15 bp, artificial
  oligonucleotide probes or primers or compounds
cagcattggtattca

SEQ ID NO: 21 - DNA, 18 bp, artificial
  oligonucleotide probes or primers or compounds
cagcttttaaccagagtg

SEQ ID NO: 22 - DNA, 19 bp, artificial
  Oligonucleotide nucleobase sequence
tgtctagaatgcgcacgtc

SEQ ID NO: 23 - DNA, 19 bp, artificial
  Oligonucleotide nucleobase sequence
tgtctagagcctgcacgtc

SEQ ID NO: 24 - DNA, 19 bp, artificial
  Oligonucleotide nucleobase sequence
tgtctagcatgtgcacgtc

SEQ ID NO: 25 - DNA, 19 bp, artificial
  Oligonucleotide nucleobase sequence
tgtctagatgtagcacgtc

SEQ ID NO: 26 - DNA, 19 bp, artificial
  Oligonucleotide nucleobase sequence
tgtctagcgtacgcacgtc

SEQ ID NO: 27 - DNA, 19 bp, artificial
  Oligonucleotide nucleobase sequence
tgtctaggaatggcacgtc

SEQ ID NO: 28 - DNA, 19 bp, artificial
  Oligonucleotide nucleobase sequence
tgtctaggtaatgcacgtc

SEQ ID NO: 29 - DNA, 19 bp, artificial
  Oligonucleotide nucleobase sequence
tgtctaggcgtggcacgtc

SEQ ID NO: 30 - DNA, 19 bp, artificial
  Oligonucleotide nucleobase sequence
tgtctagacctagcacgtc

SEQ ID NO: 31 - DNA, 19 bp, artificial
  Oligonucleotide nucleobase sequence
tgtctagcaactgcacgtc

SEQ ID NO: 32 - DNA, 19 bp, artificial
  Oligonucleotide nucleobase sequence
tgtctagattcagcacgtc

SEQ ID NO: 33 - DNA, 19 bp, artificial
  Oligonucleotide nucleobase sequence
tgtctaggtcctgcacgtc

SEQ ID NO: 34 - DNA, 19 bp, artificial
  Oligonucleotide nucleobase sequence
tgtctagacttggcacgtc

SEQ ID NO: 35 - DNA, 19 bp, artificial
  Oligonucleotide nucleobase sequence
tgtctagacgctgcacgtc

SEQ ID NO: 36 - DNA, 80 bp, artificial
  Oligonucleotide nucleobase sequence
  misc_feature (17)..(20): n is a, c, g, or t
ccgcaagtggcgtgatnnnnagatcggaagagcgtcgtgtagtccgcatgtcgcgtgatagggatatcttgcgggacgtg

SEQ ID NO: 37 - DNA, 80 bp, artificial
  Oligonucleotide nucleobase sequence
  misc_feature (17)..(20): n is a, c, g, or t
ccgcaagtggacatcgnnnnagatcggaagagcgtcgtgtagtccgcatgtcgcgtgatagggatatcttgcgggacgtg

SEQ ID NO: 38 - DNA, 80 bp, artificial
  Oligonucleotide nucleobase sequence
  misc_feature (17)..(20): n is a, c, g, or t
ccgcaagtgggcctaannnnagatcggaagagcgtcgtgtagtccgcatgtcgcgtgatagggatatcttgcgggacgtg

SEQ ID NO: 39 - DNA, 80 bp, artificial
  Oligonucleotide nucleobase sequence
  misc_feature (17)..(20): n is a, c, g, or t
ccgcaagtggtggtcannnnagatcggaagagcgtcgtgtagtccgcatgtcgcgtgatagggatatcttgcgggacgtg

SEQ ID NO: 40 - DNA, 80 bp, artificial
  Oligonucleotide nucleobase sequence
  misc_feature (17)..(20): n is a, c, g, or t
ccgcaagtggcactgtnnnnagatcggaagagcgtcgtgtagtccgcatgtcgcgtgatagggatatcttgcgggacgtg

SEQ ID NO: 41 - DNA, 80 bp, artificial
  Oligonucleotide nucleobase sequence
  misc_feature (17)..(20): n is a, c, g, or t
ccgcaagtggattggcnnnnagatcggaagagcgtcgtgtagtccgcatgtcgcgtgatagggatatcttgcgggacgtg

SEQ ID NO: 42 - DNA, 21 bp, artificial
  Oligonucleotide nucleobase sequence
ccctatgacgcgacatgcgga

SEQ ID NO: 43 - DNA, 55 bp, artificial
  Oligonucleotide nucleobase sequence
ccgcaaggagatcggaagagcacacgtctgaactccatatgcttgcggcgtctac

SEQ ID NO: 44 - DNA, 58 bp, artificial; identical to SEQ ID NO: 18
  Oligonucleotide nucleobase sequence

SEQ ID NO: 45 - DNA, 61 bp, artificial
  Oligonucleotide nucleobase sequence
caagcagaagacggcatacgagatcggtctcggcattcctgctgaaccgctcttccgatct

SEQ ID NO: 46 - DNA, 19 bp, artificial
  Oligonucleotide nucleobase sequence
tgtctagtgccagcacgtc

SEQ ID NO: 47 - DNA, 25 bp, artificial
  Oligonucleotide nucleobase sequence
starycgtctacatccacccacgtc

SEQ ID NO: 48 - DNA, 25 bp, artificial
  Oligonucleotide nucleobase sequence
starycgtctacgcttgtccacgtc

SEQ ID NO: 49 - DNA, 25 bp, artificial
  Oligonucleotide nucleobase sequence
chstrcgtctacggacttccacgtc

SEQ ID NO: 50 - DNA, 25 bp, artificial
  Oligonucleotide nucleobase sequence
chstrcgtctacaagtccccacgtc

SEQ ID NO: 51 - DNA, 24 bp, artificial
  Oligonucleotide nucleobase sequence
  misc_feature (4)..(4): n is a, c, g, or t
bamncgtctactctctaccacgtc

SEQ ID NO: 52 - DNA, 24 bp, artificial
  Oligonucleotide nucleobase sequence
  misc_feature (4)..(4): n is a, c, g, or t
bamncgtctacctctcgccacgtc

SEQ ID NO: 53 - DNA, 24 bp, artificial
  Oligonucleotide nucleobase sequence
bachcgtctacagttcaccacgtc

SEQ ID NO: 54 - DNA, 24 bp, artificial
  Oligonucleotide nucleobase sequence
bachcgtctacgacctgccacgtc

SEQ ID NO: 55 - DNA, 24 bp, artificial
  Oligonucleotide nucleobase sequence
bacdcgtctaccaagctccacgtc

SEQ ID NO: 56 - DNA, 24 bp, artificial
  Oligonucleotide nucleobase sequence
bacdcgtctactggatcccacgtc

SEQ ID NO: 57 - DNA, 20 bp, artificial
  Oligonucleotide nucleobase sequence
cgtctacccaagtccacgtc

SEQ ID NO: 58 - DNA, 20 bp, artificial
  Oligonucleotide nucleobase sequence
cgtctacttggacccacgtc

SEQ ID NO: 59 - DNA, 20 bp, artificial
  Oligonucleotide nucleobase sequence
cgtctacggcttaccacgtc

SEQ ID NO: 60 - DNA, 24 bp, artificial
  Oligonucleotide nucleobase sequence
tchrcgtctacccgcggccacgtc

SEQ ID NO: 61 - DNA, 24 bp, artificial
  Oligonucleotide nucleobase sequence
tchrcgtctacttataaccacgtc

SEQ ID NO: 62 - DNA, 80 bp, artificial; identical to SEQ ID NO: 36
  Oligonucleotide nucleobase sequence
  misc_feature (17)..(20): n is a, c, g, or t

SEQ ID NO: 63 - DNA, 80 bp, artificial; identical to SEQ ID NO: 37
  Oligonucleotide nucleobase sequence
  misc_feature (17)..(20): n is a, c, g, or t

SEQ ID NO: 64 - DNA, 80 bp, artificial; identical to SEQ ID NO: 38
  Oligonucleotide nucleobase sequence
  misc_feature (17)..(20): n is a, c, g, or t

SEQ ID NO: 65 - DNA, 80 bp, artificial; identical to SEQ ID NO: 39
  Oligonucleotide nucleobase sequence
  misc_feature (17)..(20): n is a, c, g, or t

SEQ ID NO: 66 - DNA, 80 bp, artificial; identical to SEQ ID NO: 40
  Oligonucleotide nucleobase sequence
  misc_feature (17)..(20): n is a, c, g, or t

SEQ ID NO: 67 - DNA, 80 bp, artificial; identical to SEQ ID NO: 41
  Oligonucleotide nucleobase sequence
  misc_feature (17)..(20): n is a, c, g, or t

SEQ ID NO: 68 - DNA, 80 bp, artificial
  Oligonucleotide nucleobase sequence
  misc_feature (17)..(20): n is a, c, g, or t
ccgcaagtgggatctgnnnnagatcggaagagcgtcgtgtagtccgcatgtcgcgtgatagggatatcttgcgggacgtg

SEQ ID NO: 69 - DNA, 80 bp, artificial
  Oligonucleotide nucleobase sequence
  misc_feature (17)..(20): n is a, c, g, or t
ccgcaagtggtcaagtnnnnagatcggaagagcgtcgtgtagtccgcatgtcgcgtgatagggatatcttgcgggacgtg

SEQ ID NO: 70 - DNA, 80 bp, artificial
  Oligonucleotide nucleobase sequence
  misc_feature (17)..(20): n is a, c, g, or t
ccgcaagtggagcgctnnnnagatcggaagagcgtcgtgtagtccgcatgtcgcgtgatagggatatcttgcgggacgtg

SEQ ID NO: 71 - DNA, 80 bp, artificial
  Oligonucleotide nucleobase sequence
  misc_feature (17)..(20): n is a, c, g, or t
ccgcaagtgggatatcnnnnagatcggaagagcgtcgtgtagtccgcatgtcgcgtgatagggatatcttgcgggacgtg

SEQ ID NO: 72 - DNA, 80 bp, artificial
  Oligonucleotide nucleobase sequence
  misc_feature (17)..(20): n is a, c, g, or t
ccgcaagtggcgcagannnnagatcggaagagcgtcgtgtagtccgcatgtcgcgtgatagggatatcttgcgggacgtg

SEQ ID NO: 73 - DNA, 80 bp, artificial
  Oligonucleotide nucleobase sequence
  misc_feature (17)..(20): n is a, c, g, or t
ccgcaagtggtatgagnnnnagatcggaagagcgtcgtgtagtccgcatgtcgcgtgatagggatatcttgcgggacgtg

SEQ ID NO: 74 - DNA, 80 bp, artificial
  Oligonucleotide nucleobase sequence
  misc_feature (17)..(20): n is a, c, g, or t
ccgcaagtggaggtgcnnnnagatcggaagagcgtcgtgtagtccgcatgtcgcgtgatagggatatcttgcgggacgtg

SEQ ID NO: 75 - DNA, 80 bp, artificial
  Oligonucleotide nucleobase sequence
  misc_feature (17)..(20): n is a, c, g, or t
ccgcaagtgggaacatnnnnagatcggaagagcgtcgtgtagtccgcatgtcgcgtgatagggatatcttgcgggacgtg

SEQ ID NO: 76 - DNA, 80 bp, artificial
  Oligonucleotide nucleobase sequence
  misc_feature (17)..(20): n is a, c, g, or t
ccgcaagtggacatagnnnnagatcggaagagcgtcgtgtagtccgcatgtcgcgtgatagggatatcttgcgggacgtg

SEQ ID NO: 77 - DNA, 80 bp, artificial
  Oligonucleotide nucleobase sequence
  misc_feature (17)..(20): n is a, c, g, or t
ccgcaagtgggtgcgannnnagatcggaagagcgtcgtgtagtccgcatgtcgcgtgatagggatatcttgcgggacgtg

SEQ ID NO: 78 - DNA, 80 bp, artificial
  Oligonucleotide nucleobase sequence
  misc_feature (17)..(20): n is a, c, g, or t
ccgcaagtggccaacannnnagatcggaagagcgtcgtgtagtccgcatgtcgcgtgatagggatatcttgcgggacgtg

SEQ ID NO: 79 - DNA, 80 bp, artificial
  Oligonucleotide nucleobase sequence
  misc_feature (17)..(20): n is a, c, g, or t
ccgcaagtggttggtgnnnnagatcggaagagcgtcgtgtagtccgcatgtcgcgtgatagggatatcttgcgggacgtg

SEQ ID NO: 80 - DNA, 80 bp, artificial
  Oligonucleotide nucleobase sequence
  misc_feature (17)..(20): n is a, c, g, or t
ccgcaagtggcgcggtnnnnagatcggaagagcgtcgtgtagtccgcatgtcgcgtgatagggatatcttgcgggacgtg

SEQ ID NO: 81 - DNA, 80 bp, artificial
  Oligonucleotide nucleobase sequence
  misc_feature (17)..(20): n is a, c, g, or t
ccgcaagtggtataacnnnnagatcggaagagcgtcgtgtagtccgcatgtcgcgtgatagggatatcttgcgggacgtg

SEQ ID NO: 82 - DNA, 80 bp, artificial
  Oligonucleotide nucleobase sequence
  misc_feature (17)..(20): n is a, c, g, or t
ccgcaagtggaaggatnnnnagatcggaagagcgtcgtgtagtccgcatgtcgcgtgatagggatatcttgcgggacgtg

SEQ ID NO: 83 - DNA, 80 bp, artificial
  Oligonucleotide nucleobase sequence
  misc_feature (17)..(20): n is a, c, g, or t
ccgcaagtgggcaagcnnnnagatcggaagagcgtcgtgtagtccgcatgtcgcgtgatagggatatcttgcgggacgtg

SEQ ID NO: 84 - DNA, 80 bp, artificial
  Oligonucleotide nucleobase sequence
  misc_feature (17)..(20): n is a, c, g, or t
ccgcaagtggtcgtgannnnagatcggaagagcgtcgtgtagtccgcatgtcgcgtgatagggatatcttgcgggacgtg

SEQ ID NO: 85 - DNA, 80 bp, artificial
  Oligonucleotide nucleobase sequence
  misc_feature (17)..(20): n is a, c, g, or t
ccgcaagtggctacagnnnnagatcggaagagcgtcgtgtagtccgcatgtcgcgtgatagggatatcttgcgggacgtg

SEQ ID NO: 86 - DNA, 80 bp, artificial
  Oligonucleotide nucleobase sequence
  misc_feature (17)..(20): n is a, c, g, or t
ccgcaagtggatattcnnnnagatcggaagagcgtcgtgtagtccgcatgtcgcgtgatagggatatcttgcgggacgtg

SEQ ID NO: 87 - DNA, 80 bp, artificial
  Oligonucleotide nucleobase sequence
  misc_feature (17)..(20): n is a, c, g, or t
ccgcaagtgggcgcctnnnnagatcggaagagcgtcgtgtagtccgcatgtcgcgtgatagggatatcttgcgggacgtg

SEQ ID NO: 88 - DNA, 80 bp, artificial
  Oligonucleotide nucleobase sequence
  misc_feature (17)..(20): n is a, c, g, or t
ccgcaagtggactctannnnagatcggaagagcgtcgtgtagtccgcatgtcgcgtgatagggatatcttgcgggacgtg

SEQ ID NO: 89 - DNA, 80 bp, artificial
  Oligonucleotide nucleobase sequence
  misc_feature (17)..(20): n is a, c, g, or t
ccgcaagtgggtctcgnnnnagatcggaagagcgtcgtgtagtccgcatgtcgcgtgatagggatatcttgcgggacgtg

SEQ ID NO: 90 - DNA, 80 bp, artificial
  Oligonucleotide nucleobase sequence
  misc_feature (17)..(20): n is a, c, g, or t
ccgcaagtggaagacgnnnnagatcggaagagcgtcgtgtagtccgcatgtcgcgtgatagggatatcttgcgggacgtg

SEQ ID NO: 91 - DNA, 80 bp, artificial
  Oligonucleotide nucleobase sequence
  misc_feature (17)..(20): n is a, c, g, or t
ccgcaagtggcgagtannnnagatcggaagagcgtcgtgtagtccgcatgtcgcgtgatagggatatcttgcgggacgtg

SEQ ID NO: 92 - DNA, 80 bp, artificial
  Oligonucleotide nucleobase sequence
  misc_feature (17)..(20): n is a, c, g, or t
ccgcaagtggaaccgcnnnnagatcggaagagcgtcgtgtagtccgcatgtcgcgtgatagggatatcttgcgggacgtg

SEQ ID NO: 93 - DNA, 80 bp, artificial
  Oligonucleotide nucleobase sequence
  misc_feature (17)..(20): n is a, c, g, or t
ccgcaagtgcggttatnnnnagatcggaagagcgtcgtgtagtccgcatgtcgcgtgatagggatatcttgcgggacgtg

SEQ ID NO: 94 - DNA, 55 bp, artificial; identical to SEQ ID NO: 43
  Oligonucleotide nucleobase sequence

SEQ ID NO: 95 - DNA, 60 bp, artificial
  Oligonucleotide nucleobase sequence
caagcagaagacggcatacgagatcggtctcgggagttcagacgtgtgctcttccgatct

SEQ ID NO: 96 - DNA, 26 bp, artificial
  oligonucleotide probes or primers or compounds
gttcagagttctacagtccgacgatc

SEQ ID NO: 97 - DNA, 40 bp, artificial
  oligonucleotide probes or primers or compounds
  misc_feature (1)..(6): n is a, c, g, or t
nnnnnngatcgtcggttcagagttctacagtccgacgatc

SEQ ID NO: 98 - DNA, 40 bp, artificial; identical to SEQ ID NO: 97
  oligonucleotide probes or primers or compounds
  misc_feature (1)..(6): n is a, c, g, or t

SEQ ID NO: 99 - DNA, 39 bp, artificial
  oligonucleotide probes or primers or compounds
  misc_feature (1)..(6): n is a, c, g, or t
nnnnnngatcgcggttcagagttctacagtccgacgatc

SEQ ID NO: 100 - DNA, 40 bp, artificial; identical to SEQ ID NO: 97
  oligonucleotide probes or primers or compounds
  misc_feature (1)..(6): n is a, c, g, or t

SEQ ID NO: 101 - DNA, 49 bp, artificial
  oligonucleotide probes or primers or compounds
  misc_feature (10)..(12): n is a, c, g, or t
  misc_feature (44)..(49): n is a, c, g, or t
ctatccagcnnntggaattctcgggtgccaaggagctggatagnnnnnn

SEQ ID NO: 102 - DNA, 22 bp, artificial
  oligonucleotide probes or primers or compounds
gccttggcacccgagaattcca

SEQ ID NO: 103 - DNA, 50 bp, artificial
  oligonucleotide probes or primers or compounds
aatgatacggcgaccaccgagatctacacgttcagagttctacagtccga

SEQ ID NO: 104 - DNA, 63 bp, artificial
  oligonucleotide probes or primers or compounds
caagcagaagacggcatacgagattacaaggtgactggagttccttggcacccgagaattcca

SEQ ID NO: 105 - DNA, 63 bp, artificial
  oligonucleotide probes or primers or compounds
caagcagaagacggcatacgagataggaatgtgactggagttccttggcacccgagaattcca

SEQ ID NO: 106 - DNA, 63 bp, artificial
  oligonucleotide probes or primers or compounds
caagcagaagacggcatacgagatcttttggtgactggagttccttggcacccgagaattcca

SEQ ID NO: 107 - DNA, 63 bp, artificial
  oligonucleotide probes or primers or compounds
caagcagaagacggcatacgagattagttggtgactggagttccttggcacccgagaattcca

SEQ ID NO: 108 - DNA, 63 bp, artificial
  oligonucleotide probes or primers or compounds
caagcagaagacggcatacgagatccggtggtgactggagttccttggcacccgagaattcca

SEQ ID NO: 109 - DNA, 63 bp, artificial
  oligonucleotide probes or primers or compounds
caagcagaagacggcatacgagatgtagccgtgactggagttccttggcacccgagaattcca

SEQ ID NO: 110 - DNA, 16 bp, artificial
  oligonucleotide probes or primers or compounds
  misc_feature (1)..(16): n is a, c, g, or t
nnnnnnnnnnnnnnnn

SEQ ID NO: 111 - DNA, 14 bp, artificial
  oligonucleotide probes or primers or compounds
agcattggtattca

SEQ ID NO: 112 - DNA, 79 bp, artificial
  capture probe oligonucleotide
  misc_feature (17)..(20): n is a, c, g, or t
  misc_feature (75)..(79): n is a, c, g, or t
ccgcaattgggcctaannnnagatcggaagagcgtcgtgtagtccgcatgtcgcgtgatagggatatcttgcggnnnnn

SEQ ID NO: 113 - DNA, 79 bp, artificial; identical to SEQ ID NO: 112
  capture probe oligonucleotide
  misc_feature (17)..(20): n is a, c, g, or t
  misc_feature (75)..(79): n is a, c, g, or t

(19) United States
(12) Patent Application Publication
(10) Pub. No.: US 2021/0071247 A1
(43) Pub. Date: Mar. 11, 2021

(54) MASSIVELY PARALLEL DISCOVERY METHODS FOR OLIGONUCLEOTIDE THERAPEUTICS

(71) Applicant: Roche Innovation Center Copenhagen A/S, Hørsholm (DK)

(72) Inventors: Mads JENSEN, Farum (DK); Jonas VIKESAA, Kirke Hyllinge (DK); Lukasz KIELPINSKI, Frederiksberg (DK); Lars JOENSON, Viby Sjaelland (DK); Filippo SLADOJEVICH, Rheinfelden (CH)

(73) Assignee: Roche Innovation Center Copenhagen A/S, Hørsholm (DK)

(21) Appl. No.: 17/053,690

(22) PCT Filed: May 6, 2019
(86) PCT No.: PCT/EP2019/061515

§ 371 Date: Nov. 6, 2020

(30) Foreign Application Priority Data
May 7, 2018 (EP) 18171022.9
May 7, 2018 (EP) 18171024.5
May 7, 2018 (EP) 18171029.4

(51) Int. Cl. C12Q 1/6869; C12Q 1/6806

(52) U.S. Cl. CPC C12Q 1/6869; C12Q 1/6806

(57) ABSTRACT

The invention relates to the field of therapeutic oligonucleotide analytics and discovery, and provides methods for primer based parallel sequencing of modified oligonucleotides which provide sequence based quality information which may be used in oligonucleotide therapeutic discovery, manufacture, quality assurance, therapeutic development, and patient monitoring.

### FIELD OF INVENTION

The invention relates to the field of therapeutic oligonucleotide analytics and discovery, and provides methods for primer based parallel sequencing of modified oligonucleotides which provide sequence based quality information which may be used in oligonucleotide therapeutic discovery, manufacture, quality assurance, therapeutic development, and patient monitoring.

### BACKGROUND

EP 1 914 317 A1 discloses a method for the qualitative and quantitative detection of short nucleic acid sequences of about 8 to 50 nucleotides in length. The method employs a hybridization of a overlapping capture probe and polymerase elongation. The example of EP′317 uses a DNA phosphorothioate oligonucleotide G3139.

WO 01/34845 A1 discloses a method for quantitating phosphorothioate oligonucleotides from a bodily fluid or extract. The method employs a capture probe which partially hybridizes to the oligonucleotide, followed by enzymatic labelling of the capture probe/oligonucleotide hybride, followed by detection of the label. The examples of WO′845 uses a DNA phosphorothioate oligonucleotide ISIS 2302.

Caifu et al., NAR 33 (2005) E179 discloses the detection and quantification of un-modified short oligonucleotides such as microRNAs using a capture probe/PCR based amplification system.

Froim et al., VAR (1995) 4219-4223 discloses a method for phosphorothioate antisense DNA sequencing by capillary electrophoresis with UV detection.

Tremblay et al., Bioanalysis (2011) 3(5) discloses a dual ligation based hybridization assay for the specific determination of oligonucleotide therapeutics and for use to specifically determine individual metabolites in complex mixtures implementing quantitative PCR. WO2007/025281 discloses a method for detecting a short oligonucleotide using a capture probe hybridization, ligation and amplification.

Cheng et al., Molecular Therapey Nucleic Acids (2013) e67 discloses on in vivo selex for identification of brain-penetrating aptamers. The aptamers are 2′fluoro modified phosphodiester oligonucleotides which are sequenced using Sanger sequencing or Illumina sequencing using OneStep RT-PCR kit (Qiagen) or Superscript III Reverse transcriptase for first strand synthesis.

Crouzier et al., PLoS ONE (2012) e359900 refers to efficient reverse transcription of locked nucleic acid nucleotides using Superscript III to generate nuclease resistant RNA aptamers. Crouzier et al uses Sanger based sequencing to sequence PCR amplification products obtained from first strand synthesis of LNA aptamer oligonucleotides. Notably the LNA aptamers had single LNA nucleosides in an otherwise RNA phosphodiester nucleoside background.

The present inventors have provided 3′ capture probe ligation and polymerase based detection of modified oligonucleotides, such as 2′-O-MOE and LNA modified oligonucleotides which enable massively parallel sequencing of such modified oligonucleotides. Previously, as described in PCT/EP2017/078695, the inventors have developed a method for detection, quantification, amplification, sequencing or cloning of the nucleoside modified oligonucleotides, such as LNA modified oligonucleotides, based upon the 3′ capture of the modified oligonucleotide using a capture probe, followed by chain elongation and detection, quantification, amplification, sequencing or cloning of the nucleoside modified oligonucleotides. PCT/EP2017/078695 discloses the use of Volcano2G polymerase as a suitable enzyme for chain elongation.

The efficiency of the methods developed by the present inventors not only enable modified oligonucleotide capture and PCR based detection, but for the first time enable massively parallel sequencing of modified oligonucleotide samples, a revolutionary technique which provides a key new tool in oligonucleotide therapeutic discovery, manufacture, quality assurance, therapeutic development, and patient monitoring.

### SUMMARY OF THE INVENTION

The invention provides for a method for sequencing a modified oligonucleotide such as 2′ sugar modified oligonucleotide, such as a LNA oligonucleotide or a 2-O-methoxyethyl oligonucleotide, said method comprising the sequential steps of:

(I) Perform polymerase mediated first strand synthesis of an oligonucleotide template comprising the modified oligonucleotide to produce a nucleic acid sequence comprising the complement of the modified oligonucleotide;

(II) Perform primer based sequencing of the first strand synthesis product of step (I).

The invention provides for a method for parallel sequencing a population of modified oligonucleotides, such as a 2′ sugar modified oligonucleotides, such as a population of LNA oligonucleotides or a population of 2-O-methoxyethyl oligonucleotides, said method comprising the steps of:

(I) Perform polymerase mediated first strand synthesis of a population of oligonucleotide templates, wherein each member of the population is or comprises a modified oligonucleotide, to produce a library of nucleic acid sequences, each comprising the complement of a modified oligonucleotide present in the population of oligonucleotides;

(II) Perform primer based sequencing of the population of first strand synthesis products of step (I).

After step (I) and prior to step (II) an PCR amplification step may be performed: In some embodiments the primer based sequencing step may comprise clonal amplification of the first strand synthesis products prior to or as part of step (II). In some embodiments the clonal amplification employs solid phase amplification or emulsion phase amplification.

The invention provides for a method for sequencing the nucleobase sequence of a modified oligonucleotide said method comprising the steps of:
a. Ligate a 5′ capture probe oligonucleotide to the 5′ terminus of the modified oligonucleotide, wherein the 5′ capture probe comprises a primer binding site;
b. Perform polymerase mediated 5′-3′ first strand synthesis from the capture probe to produce a nucleic acid sequence comprising the complement of the modified oligonucleotide;
c. Perform primer based sequencing of the first strand synthesis product obtained in step b).

This method may be used for example when the 3′ region of the modified oligonucleotide comprises a known sequence, the primer based sequencing (and/or PCR step if included) is performed using a primer which is specific for the 3′ region of the modified oligonucleotide, and a primer which is specific for the 5′ capture probe.

The invention provides for a method for sequencing the nucleobase sequence of a modified oligonucleotide said method comprising the steps of:
a. Ligate a 3′ capture probe to the 3′ terminus of the modified oligonucleotide, wherein the 3′ capture probe comprises a primer binding site
b. Ligate a 5′ capture probe to the 5′ terminus of the modified oligonucleotide, wherein the 5′ capture probe comprises a primer binding site;
c. Perform polymerase mediated 5′-3′ first strand synthesis from the capture probe to produce a nucleic acid sequence comprising the complement of the modified oligonucleotide;
d. Perform primer based sequencing of the first strand synthesis product obtained in step c);
   Wherein step a and step b may be performed in either order or simultaneously.

The primer based sequencing, and/or PCR step if performed by using a primer which is specific for the 3′ capture probe and a primer which is specific for the 5′ capture probe.

The invention provides for a method for parallel sequencing the nucleobase sequences of a population of modified oligonucleotides said method comprising the steps of:
a. Ligate a 5′ capture probe oligonucleotide to the 5′ terminus of the population of modified oligonucleotides, wherein the 5′ capture probe comprises a primer binding site;
b. Perform polymerase mediated 5′-3′ first strand synthesis from the capture probe to produce a population of nucleic acid sequences each comprising the complement of a member of the population of modified oligonucleotides;
c. Perform primer based parallel sequencing of the first strand synthesis products obtained in step b);

Wherein step a and step b may be performed in either order or simultaneously.

The invention provides for a method for the parallel sequencing the nucleobase sequences of a population of modified oligonucleotides said method comprising the steps of:
a. Ligate a 3′ capture probe to the 3′ terminus of the modified oligonucleotides, wherein the 3′ capture probe comprises a primer binding site
b. Ligate a 5′ capture probe to the 5′ terminus of the modified oligonucleotides, wherein the 5′ capture probe comprises a primer binding site;
c. Perform polymerase mediated 5′-3′ first strand synthesis from the capture probe to produce a population of nucleic acid sequences each comprising the complement of a modified oligonucleotide of the population of modified oligonucleotides;
d. Perform primer based parallel sequencing of the first strand synthesis products obtained in step b);

Wherein step a and step b may be performed in either order or simultaneously.

The primer based sequencing, and/or PCR step if performed by using a primer which is specific for the 3′ capture probe and a primer which is specific for the 5′ capture probe.

The invention provides for a method or the parallel sequencing of a population of modified oligonucleotides, wherein the method comprises the steps of:
a. Ligating a 3′ capture probe oligonucleotide to the 3′ terminus of a population of modified oligonucleotides, wherein either the 3′ capture probe comprises a first primer binding site or is a self-priming 3′capture probe;
b. Ligating a 5′ capture probe to the 5′ terminus of the population of modified oligonucleotides;
c. Perform first strand synthesis form the 3′ capture probes;
d. Perform PCR amplification of the first strands obtained in step c, using a pair of PCR primers, one which is specific for the 3′ capture probe oligonucleotide, the other which is specific for the 5′ capture probe to prepare a population of amplification products;
e. Perform primer based parallel sequencing of the PCR amplification products of step d; wherein steps a. and b. may be performed in either order or simultaneously.

To facilitate first strand synthesis, the 3′ capture probe may comprises a first primer binding site or may be a self-priming 3′capture probe.

The invention provides for a method or the sequencing of a modified oligonucleotide, wherein the method comprises the steps of:
a. Ligating a 3′ capture probe oligonucleotide to the 3′ terminus of the modified oligonucleotide, wherein either the 3′ capture probe comprises a first primer binding site or is a self-priming 3′capture probe;
b. Ligating a 5′ capture probe oligonucleotide to the 5′ terminus of the modified oligonucleotide;
c. Perform first strand synthesis form the 3′ capture probe
d. Perform a PCR amplification of the first strand obtained in step c, using a pair of PCR primers, one which is specific for the 3′ capture probe oligonucleotide, the other which is specific for the 5′ capture probe;
e. Perform primer based sequencing of the PCR amplification product of step wherein steps a. and b. may be performed in either order or simultaneously.

The invention provides for a method or the parallel sequencing of a population of modified oligonucleotides, wherein the method comprises the steps of:
a. Ligating a 3′ capture probe oligonucleotide to the 3′ terminus of the population of modified oligonucleotides, wherein either the 3′ capture probe comprises a first primer binding site or is a self-priming 3′capture probe;
b. Ligating a 5′ capture probe oligonucleotide to the 5′ terminus of the population of modified oligonucleotides;
c. Perform first strand synthesis form the 3′ capture probes to generate a library of first strand synthesis products;
d. Perform a PCR amplification of the library of first strand synthesis products obtained in step c, using a pair of PCR primers, one which is specific for the 3′ capture probe oligonucleotide, the other which is specific for the 5′ capture probe;
e. Perform primer based parallel sequencing of the PCR amplification products of step d. wherein steps a. and b. may be performed in either order or simultaneously.

An advantage of using 5′capture probes which are ligated prior to first strand synthesis is that it improves the sequencing signal obtained from the full length first strand synthesis complement of the modified oligonucleotide, as compared to possible truncated first strand synthesis products which may be PCR amplified or sequenced if a 5′capture probe is not used.

The invention provides for a method for sequencing the nucleobase sequence of a modified oligonucleotide said method comprising the steps of:
a. Ligate a capture probe oligonucleotide to the 3′ terminus of the modified oligonucleotide;
b. Perform polymerase mediated 5′-3′ first strand synthesis from the capture probe to produce a nucleic acid sequence comprising the complement of the modified oligonucleotide;
c. Perform primer based sequencing of the first strand synthesis product obtained in step b).

The invention provides for a method for parallel sequencing the base sequence of a population of modified oligonucleotides said method comprising the steps of:
a. Ligate a capture probe oligonucleotide to the 3′ terminus of the modified oligonucleotides present in the population of modified oligonucleotides;
b. Perform polymerase mediated 5′-3′ first strand synthesis from the capture probe to produce a population of nucleic acid sequences, each comprising the complement of base sequence of a modified oligonucleotide present in the population of modified oligonucleotides;
c. Perform primer based parallel sequencing of the population of first strand synthesis products obtained in step b).

The invention provides for a method for sequencing the nucleobase sequence of a modified oligonucleotide, such as 2′ sugar modified phosphorothioate oligonucleotide, said method comprising the steps of:
a. Ligate a capture probe oligonucleotide to the 3′ terminus of the modified oligonucleotide;
b. Perform polymerase mediated 5′-3′ first strand synthesis from the capture probe to produce a nucleic acid sequence comprising the complement of the modified oligonucleotide;
c. Ligate an adapter probe to the 3′ end of the first strand synthesis product obtained in step b; and subsequently either
Perform primer based sequencing of the ligation product obtained in step c); or
Perform PCR amplification of the ligation product obtained in step c) and perform primer based sequencing of the PCR amplification product.

The invention provides for a method for parallel sequencing the nucleobase sequence of a population of modified oligonucleotides, such as a population of 2′sugar modified phosphorothioate oligonucleotides, said method comprising the steps of:
a. Ligate a capture probe oligonucleotide to the 3′ terminus of the modified oligonucleotides present in the population of modified oligonucleotides;
b. Perform polymerase mediated 5′-3′ first strand synthesis from the capture probe to produce a population of nucleic acid sequences, each comprising the complement of base sequence of a modified oligonucleotide present in the population of modified oligonucleotides;
c. Ligate an adapter probe to the 3′ end of the first strand synthesis products obtained in step b; and subsequently either
   Perform primer based parallel sequencing of the ligation products obtained in step c); or
   Perform PCR amplification of the ligation products obtained in step c) and perform primer based parallel sequencing of the PCR amplification products.

The invention provides for a method for sequencing the nucleobase sequence of a modified oligonucleotide said method comprising the steps of:
a. Ligate a capture probe oligonucleotide to the 3′ terminus of the modified oligonucleotide;
b. Perform polymerase mediated 5′-3′ first strand synthesis from the capture probe to produce a nucleic acid sequence comprising the complement of the modified oligonucleotide;
c. Perform primer based sequencing of the first strand synthesis product obtained in step b); wherein the primer based sequencing step comprises the clonal amplification of the first strand synthesis step (b) using a clonal amplification primers, wherein one of the clonal amplification primers is specific for the 3′ capture probe, and the second clonal amplification primer is specific for the modified oligonucleotide such as a 5′ region of the modified oligonucleotide.

The invention provides for a method for parallel sequencing the base sequence of a population of modified oligonucleotides said method comprising the steps of:
a. Ligate a capture probe oligonucleotide to the 3′ terminus of the modified oligonucleotides present in the population of modified oligonucleotides;
b. Perform polymerase mediated 5′-3′ first strand synthesis from the capture probe to produce a population of nucleic acid sequences, each comprising the complement of base sequence of a modified oligonucleotide present in the population of modified oligonucleotides;
c. Perform primer based parallel sequencing of the population of first strand synthesis products obtained in step b), wherein the primer based parallel sequencing step comprises the clonal amplification of the first strand synthesis step (b) using a clonal amplification primers, wherein one of the clonal amplification primers is specific for the 3′ capture probe, and the second clonal amplification primer is specific for the modified oligonucleotides such as a 5′ region of the modified oligonucleotides.

The invention provides for a method for sequencing the nucleobase sequence of a modified oligonucleotide said method comprising the steps of:
a. Ligate a capture probe oligonucleotide to the 3′ terminus of the modified oligonucleotide;
b. Perform polymerase mediated 5′-3′ first strand synthesis from the capture probe to produce a nucleic acid sequence comprising the complement of the modified oligonucleotide;
c. Perform PCR amplification of the first strand synthesis product obtained in step b); using primers, wherein one of the PCR primers is specific for the 3′ capture probe, and the second PCR primer is specific for the modified oligonucleotide such as a 5′ region of the modified oligonucleotide.
d. Perform primer based sequencing of the PCR amplification product of step c)

The invention provides for a method for parallel sequencing the base sequence of a population of modified oligonucleotides said method comprising the steps of:
a. Ligate a capture probe oligonucleotide to the 3′ terminus of the modified oligonucleotides present in the population of modified oligonucleotides;
b. Perform polymerase mediated 5′-3′ first strand synthesis from the capture probe to produce a population of nucleic acid sequences, each comprising the complement of base sequence of a modified oligonucleotide present in the population of modified oligonucleotides;
c. Perform PCR amplification of the first strand synthesis product obtained in step b); using primers, wherein one of the PCR primers is specific for the 3′ capture probe, and the second PCR primer is specific for the modified oligonucleotide such as a 5′ region of the modified oligonucleotide.
d. Perform primer based parallel sequencing of the PCR amplification products of step c)

The invention provides for a method for sequencing the nucleobase sequence of a modified oligonucleotide said method comprising the steps of:
a. Ligate a capture probe oligonucleotide to the 3′ terminus of the modified oligonucleotide;
b. Perform polymerase mediated 5′-3′ first strand synthesis from the capture probe to produce a nucleic acid sequence comprising the complement of the modified oligonucleotide;
c. Polynucleate the 3′ terminus of the first strand synthesis product (e.g. polyadenylate).
d. Perform a second strand synthesis using a primer which comprises a sequence which is complementary to the polynucleated 3′ terminus (e.g. a second strand synthesis primer with a polyT sequence),
e. Perform primer based sequencing of the second strand synthesis product obtained in step d).

The invention provides for a method for parallel sequencing the base sequence of a population of modified oligonucleotides said method comprising the steps of:
a. Ligate a capture probe oligonucleotide to the 3′ terminus of the modified oligonucleotides present in the population of modified oligonucleotides;
b. Perform polymerase mediated 5′-3′ first strand synthesis from the capture probe to produce a population of nucleic acid sequences, each comprising the complement of base sequence of a modified oligonucleotide present in the population of modified oligonucleotides;
c. Polynucleate the 3′ terminus of the first strand synthesis products (e.g. polyadenylate).
d. Perform a second strand synthesis using a primer which comprises a sequence which is complementary to the polynucleated 3′ terminus (e.g. a second strand synthesis primer with a polyT sequence),
e. Perform primer based parallel sequencing of the second strand synthesis products obtained in step d).

The invention provides for a method for sequencing the nucleobase sequence of a modified oligonucleotide said method comprising the steps of:
a. Ligate a capture probe oligonucleotide to the 3′ terminus of the modified oligonucleotide;
b. Perform polymerase mediated 5′-3′ first strand synthesis from the capture probe to produce a nucleic acid sequence comprising the complement of the modified oligonucleotide;
c. Polynucleate the 3′ terminus of the first strand synthesis product (e.g. polyadenylate).
d. Perform a second strand synthesis using a primer which comprises a sequence which is complementary to the polynucleated 3′ terminus (e.g. a second strand synthesis primer with a polyT sequence),
e. Perform PCR amplification of the second strand synthesis product
f. Perform primer based sequencing of the PCR product obtained in step e).

The invention provides for a method for parallel sequencing the base sequence of a population of modified oligonucleotides said method comprising the steps of:
a. Ligate a capture probe oligonucleotide to the 3′ terminus of the modified oligonucleotides present in the population of modified oligonucleotides;
b. Perform polymerase mediated 5′-3′ first strand synthesis from the capture probe to produce a population of nucleic acid sequences, each comprising the complement of base sequence of a modified oligonucleotide present in the population of modified oligonucleotides;
c. Polynucleate the 3′ terminus of the first strand synthesis products (e.g. polyadenylate).
d. Perform a second strand synthesis using a primer which comprises a sequence which is complementary to the polynucleated 3′ terminus (e.g. a second strand synthesis primer with a polyT sequence),
e. Perform PCR amplification of the second strand synthesis products
f. Perform primer based sequencing of the PCR products obtained in step e).

Suitably the PCR primers may be a first PCR primer specific for the 3′ capture probe and a second PCR primer which is specific for the second strand synthesis primer (of is the second strand synthesis primer).

The invention provides for a method for determining the sequence heterogeneity in a population of modified oligonucleotides from a common oligonucleotide synthesis run, or from a pool of independent oligonucleotide synthesis runs, said method comprising the steps of:
(i) Obtain or synthesize the modified oligonucleotides,
(ii) Perform the method for parallel sequencing of the modified oligonucleotides according to the invention,
(iii) Analyse the sequence data obtained in step (ii) to identify the sequence heterogeneity of the population of modified oligonucleotides.

The invention provides for a method for the validating the sequence of a modified oligonucleotide, said method comprising the steps of:
(i) Obtain or synthesize the modified oligonucleotide
(ii) Performing the method for parallel sequencing of the modified oligonucleotide according to the invention,
(iii) Analyse the sequence data obtained to validate the sequence of the modified oligonucleotide

The invention provides for a method for the determination of the purity of a modified oligonucleotide
(i) Obtain or synthesize the modified oligonucleotide
(ii) Performing the method for parallel sequencing of the modified oligonucleotide according to the invention,
(iii) Analyse the sequence data obtained to determine the purity of the modified oligonucleotide.

The invention provides for the use of parallel sequencing such as massively parallel sequencing to sequence the nucleobase sequence of a population of modified oligonucleotides.

The invention provides for the use of sequence by synthesis sequencing to sequence the nucleobase sequence of a modified oligonucleotide.

The invention provides for the use of sequence by synthesis sequencing to sequence the nucleobase sequence of a population of modified oligonucleotides in parallel.

The invention provides for the use of sequence by synthesis sequencing to determine the quality of the product of a synthesis or manufacturing run of a modified oligonucleotide, such as a therapeutic oligonucleotide.

The invention provides for the use of sequence by synthesis sequencing to determine the heterogeneity in sequence and occurrence of each unique sequence of the products of a synthesis or manufacturing run of a modified oligonucleotide, such as a therapeutic oligonucleotide.

The invention provides for the use of sequence by synthesis sequencing to determine the quality of the product of a synthesis or manufacturing run of a modified oligonucleotide, such as a therapeutic oligonucleotide.

The invention provides for the use of sequence by synthesis sequencing to determine the heterogeneity of the product of a synthesis or manufacturing run of a modified oligonucleotide, such as therapeutic oligonucleotide.

The invention provides for the use of primer based sequencing to determine the quality of the product of a synthesis or manufacturing run of a modified oligonucleotide, such as a therapeutic oligonucleotide.

The invention provides for the use of primer based sequencing to determine the heterogeneity in sequence and occurrence of each unique sequence of the products of a synthesis or manufacturing run of a modified oligonucleotide, such as a therapeutic oligonucleotide.

The invention provides for the use of parallel sequencing such as massively parallel sequencing to determine the quality of the product of a synthesis or manufacturing run of a modified oligonucleotide, such as a therapeutic oligonucleotide.

The invention provides for the use of parallel sequencing such as massively parallel sequencing to determine the of the product of a synthesis or manufacturing run of a modified oligonucleotide, such as therapeutic oligonucleotide.

The invention provides for the use of Taq polymerase, such as SEQ ID NO 1, or a DNA polymerase enzyme with at least 70% identity to SEQ ID NO 1, such as Volcano2G polymerase, for the first strand synthesis from a template comprising a LNA modified phosphorothioate oligonucleotide or a 2′-O-methoxyethyl modified phosphorothioate oligonucleotide.

The invention provides for a method for identifying a modified oligonucleotide or modified oligonucleotide sequence which has enhanced cellular uptake in a cell said method comprising:
i. Administering a population of modified oligonucleotides wherein each member of the population of modified oligonucleotides comprises a unique nucleobase sequence to the cell;
ii. After a period of time, isolate the modified oligonucleotides from within the cell(s),
iii. Perform the parallel sequencing method of the invention on the modified oligonucleotides obtained in step (ii); to
iv. Identify one or more modified oligonucleotide sequences which are enriched in the cell or in the population of cells.

The invention provides for a method for identifying a modified oligonucleotide (sequence) which is enriched in a target tissue or cell in a mammal said method comprising:

i. Administering the mixture of modified oligonucleotides to a mammal, wherein each member of the mixture of modified oligonucleotides comprises a unique nucleobase sequence,
ii. Allow for the modified oligonucleotides to be distributed within the mammal, for example for a period of at least 6 hours;
iii. Isolate a population of modified oligonucleotides from one or more tissues or cells from the mammal, including a desired target tissue or desired target cell,
iv. Perform the parallel sequencing method of the invention on the population of modified oligonucleotides obtained in step iii, to
v. Identify the modified oligonucleotide sequences which are enriched in the desired target tissue or cell of the mammal.

The invention provides a method for identifying a modified oligonucleotide or modified oligonucleotide sequence which has enhanced cellular uptake said method comprising:

In some embodiments, the modified oligonucleotide(s) is an LNA modified oligonucleotide(s), such as a LNA phosphorothioate oligonucleotide. In some embodiments, the modified oligonucleotide(s) is an LNA modified oligonucleotide(s), such as a LNA phosphorothioate oligonucleotide, which further comprises a conjugate group, such as a N-Acetylgalactosamine (GalNAc) moiety, such as a trivalent GalNAc moiety.

In some embodiments, the modified oligonucleotide(s) is a 2′-sugar modified oligonucleotide such as a 2′-O-methoxyethyl modified oligonucleotide, such as a 2′-O-methoxyethyl phosphorothioate oligonucleotide, which may optionally further comprise a conjugate group, such as a N-Acetylgalactosamine (GalNAc) moiety, such as a trivalent GalNAc moiety.

The invention provides for a conjugate of an oligonucleotide comprising one or more 2′ modified nucleosides, such as a conjugate of an antisense oligonucleotide, such as a conjugate of an phosphorothioate antisense oligonucleotide, or a conjugate of a LNA oligonucleotide, such as an LNA gapmer or mixmer, wherein the conjugate comprises said oligonucleotide and a conjugate moiety selected from the group B to T as shown in the figures, optionally with a linker group, such as an alkyl linker positioned between the oligonucleotide and the conjugate moiety. Suitably the conjugate moiety may be positioned at the 5′ or 3′ terminus of the oligonucleotide.

### BRIEF DESCRIPTION OF FIGURES

FIG. 1 Panel A displays a schematic illustration of the two single stranded test template molecules that were generated to test the different polymerases ability to read LNA oligoes. LTT1 contains a stretch (light grey) with a LNA oligo, containing 8 LNA bases and 11 phosphorotioate backbone modifications. DTT1 is a control template comprising the same base sequence as LTT1 but using only DNA bases with phosphorodiester backbone. (B) Shows a sybr gold staining 15% TBE urea gel where the ligation reaction between the DCP1 and the oligoes LNA O1 (Lane B) and DNA O1 (Lane C) from example 1. In Lane A there was no oligo present in the ligation reaction.

FIG. 2 Panel A shows a 1D plot of the fluoresce intensities of the droplets in the 6 different Eva Green ddPCR reactions in example 2. The template molecules used for each reaction is shown above the lane of each readout. The pink line indicates the manually set threshold line separating the positive and negative droplets.

FIG. 3 displays the fluoresce intensities of the droplets in the different Eva Green ddPCR reactions performed in example 3. The enzymes used to generate the 1 strand copy at 42 C for 1 h on LTT1 is indicated above the plot (the 6 lane to the left). The 6 lanes to the right are control reaction were the ddPCR was run directly on the template without a 1. Strand synthesis. The templates used is indicated above the plot.

FIG. 4 displays the fluoresence intensities of the droplets in evagreen ddPCR on the LTT1 template with presence of different additives in different concentrations from example 4. The additives and there concentration is indicated on the plot. The concentrations of the additives are indicated above the plot. Panel E displays the quantification of the LTT1 detected in the reactions shown in panel A-D. Panel G shows the fluoresce intensities of the droplets in evagreen ddPCR on the LTT1 template with the presence of 9% PEG and an increasing amount of 1,2-Propanediol. Panel H displays the quantification of the number of positive droplets in panel G, illustrating that further addition of 1.2-propanediol doesn't increase the number of positive droplets.

FIG. 5 displays the results of the ddPCR reaction on the 1.strand synthesis on LTT1 from example 5. FIG. 5 panel A displays the ddPCR reaction on 1.strand Taq polymerase synthesis without PCR additives. The number of PCR cycles is indicated below the plot of each reaction. FIG. 5 panel B displays the results of the ddPCR reaction when 10% PEG and 0.31M was present during the 1. The number of PCR cycles is indicated below the plot of each reaction. Strand synthesis reaction. FIG. 5 panel C show the same reaction but without Taq Polymerase presence. The number of PCR cycles is indicated below the plot of each reaction. FIG. 5 panel E and F displays the ddPCR on the 1.strand synthesis reaction with phusion DNA polymerase in HF buffer with and without the 10% PEG and 0.31M 1.3-Propanediol additives. FIG. 5 panel D displays a quantification of the number of detected LTT1 copies for the 7 tested conditions (A; B; C; D; E; F).

FIG. 6 Panel A shows the sybr gold staining 15% TBE urea gel with the separation of the ligation reactions between the individual capture probes and oligoes described in example 6. The white square indicate the area cut from the gel that contains the ligated product. Panel B-E displays the top 10 most frequent 18 base pair reads for each of the four capture probes following the data processing described in example 6. The sequence of the input LNA oligo is shown above each table.

FIG. 7. Key design features of 4 exemplary Capture Probes (i)-(iv) for capturing, e.g. a sugar modified such as an LNA oligonucleotide, or a stereodefined oligonucleotide:

Region A: 5′ end is phosphorylated to enable ligation. Region A forms a first duplex with region G (forming a non linear capture probe). Regions G and A base pair to make intracellular loop, stabilizing the positioning the target modified oligonucelotide towards the 5′phosphate to enhance ligation.

Region B comprises a reaction bar code and is optional although highly advantageous for parallel sequencing. Region C may comprise a region of degenerate nucleotides or universal bases and may optionally be used, e.g. as a molecular bar code. Region B and C may be in either order.

Region D is advantageous for next generation sequencing applications using e.g. solid phase primers and is used to hybridise the ligation products or PCR amplification products to the sequencing platform (e.g. flow cell binding primers). Alternatively, if a PCR amplification step is included, PCR primers comprising the binding sites for the sequencing platform may be used. Region D may also be used as the first primer binding site.

Region E is an optional first primer binding site, and may be overlapping with region D. Region H is a region of 3′ nucleotides which hybridise with the 3′ end of the modified oligonucleotide, thereby positioning the modified oligonucleotide of ligation to the 5′ end of the capture probe. Region H may be a degenerate sequence or may be a predetermined sequence as described herein. The 3′ end of the capture probe is blocked for ligation to avoid self-ligation. A 3′ amino modification is exemplified herein, but other 3′ blocking groups may be used.

Region F′ shows the embodiment where the capture probe is self-priming via virtue of s cleavable linkage within a duplex region positioned down-stream of region D (or may be overlapping with region D.

The thin lines represent optional nucleosides connecting the regions illustrated, and as described herein these may be replaced with non-nucleosidic linkers.

FIG. 8 Panel A shows the sybr gold staining 15% TBE urea gel with the separation of the ligation reactions between the individual capture probes and oligoes described in example 7. The white square indicate the area cut from the gel that contains the ligated product. Panel B-E displays the top 10 most frequent 18 base pair reads for each of the four capture probes following the data processing described in example 7. The sequence of the input LNA oligo is shown above each table.

FIG. 9 displays the top 10 most frequent 15 base pair reads of the reaction described in example 8.

FIG. 10 panel A displays the fluorescence intensities of the droplets in the EvaGreen ddPCR reactions performed on the 1×45 min 1 strand synthesis reaction. The quantification of detected copies is shown in FIG. 10 panel B displaying the concentration in copies per ul reaction. FIG. 10 panel C displays the fluorescence intensities of the droplets in the EvaGreen ddPCR reactions performed on the reaction done with 1 3 or 5 rounds of 1. Strand synthesis. The quantification of detected copies in each reaction are show in FIG. 10 panel D displaying the concentration in copies per ul reaction.

FIG. 11. Image of gel electrophoresis of samples described in example 9. Table above the gel images describes: (1) in a top row, which capture probe was used; (2) in a bottom row, which master mix was used. Detection of the fluorescence of the fluorophore attached to the capture probes.

FIG. 12. The same gels as shown in FIG. 10, but stained with SYBR Gold.

FIG. 13. Image of gel electrophoresis of ligation samples described in example 10. Detection of the fluorescence of the fluorophore attached to the capture probes.

FIG. 14. Image of gel electrophoresis of ligation reactions described in example 11. Products indicated by arrows correspond to LNA-oligonucleotides covalently bound with both a4 and C5 capture probes. Detection of the fluorescence of the fluorophore attached to the capture probes.

FIG. 15. Bioanalyzer DNA High Sensitivity traces of PCR amplified samples described in example 11. Bioanalyzer traces display two predominant peaks, one for empty sequencing library (marked as E on the plots), derived from direct ligation of capture probes C5 and a4, and filled sequencing library (marked as F on the plots), which includes the sequence of the assayed LNA-oligonucleotide.

FIG. 16: A 5′capture probe of the invention: I. a first region comprising a 3′terminal RNA nucleoside or a 3′ terminal region of RNA nucleotides II. a second region of nucleotides [comprises the complement of a primer binding site] III. Optional third linker region (which may block polymerase read through or may be a discontinuity IV. a forth region of nucleotides. V. a fifth region of nucleotides which are complementary to the 5′ region of the modified oligonucleotide, which may optionally be a region of degenerate nucleotides & a 5′ blocking group—i.e. a 5′ group other than 5′ phosphate.

FIG. 17. Fold liver enrichment relative to unconjugated oligonucleotide (SEQ ID 35) 4 h after subcutaneous injection. GalNAc conjugated oligonucleotide (SEQ ID 22) as well as SEQ ID 26 show 3.5-fold liver enrichment compared to the unconjugated oligonucleotide (SEQ ID 35).

FIG. 18. Plasma enrichment relative to unconjugated oligo compound SEQ ID 35, 4 h after subcutaneous injection. Oligonucleotide with C16 fatty acid conjugation (SEQ ID 46) showed 12.5-fold plasma abundance compared to Naked oligonucleotide SEQ ID 35. GalNAc conjugated oligonucleotide (SEQ ID 22) showed depletion from plasma.

### DEFINITIONS

Oligonucleotide

The term “oligonucleotide” as used herein is defined as it is generally understood by the skilled person as a molecule comprising two or more covalently linked nucleosides. Such covalently bound nucleosides may also be referred to as nucleic acid molecules or oligomers. Oligonucleotides are commonly made in the laboratory by solid-phase chemical synthesis followed by purification. When referring to a sequence of the oligonucleotide, reference is made to the sequence or order of nucleobase moieties, or modifications thereof, of the covalently linked nucleotides or nucleosides. In the context of the present invention, oligonucleotides are man-made, and are chemically synthesized, and may be purified or isolated.

Modified Oligonucleotide

The term modified oligonucleotide describes an oligonucleotide comprising one or more sugar-modified nucleosides and/or modified internucleoside linkages and/or the presence of a conjugate moiety.

In some embodiments, the modified oligonucleotide is a therapeutic oligonucleotide.

In some embodiments the modified oligonucleotide comprises at least two contiguous 2′sugar modified nucleosides. In some embodiments the modified oligonucleotide comprises at least two contiguous 2′sugar modified nucleosides, independently selected from the group consisting of LNA and 2′-O-methoxyethyl nucleosides. In some embodiments the modified oligonucleotide comprises at least two contiguous LNA nucleosides. In some embodiments the modified oligonucleotide comprises at least two contiguous 2′-O-methoxyethyl nucleosides. In some embodiments the modified oligonucleotide comprises at least three contiguous 2′sugar modified nucleosides, independently selected from the group consisting of LNA and 2′-O-methoxyethyl nucleosides.

In some embodiments the modified oligonucleotide comprises at least three contiguous LNA nucleosides. In some embodiments the modified oligonucleotide comprises at least three contiguous 2′-O-methoxyethyl nucleosides. In some embodiments the modified oligonucleotide comprises at least four contiguous 2′sugar modified nucleosides, independently selected from the group consisting of LNA and 2′-O-methoxyethyl nucleosides. In some embodiments the modified oligonucleotide comprises at least four contiguous LNA nucleosides. In some embodiments the modified oligonucleotide comprises at least four contiguous 2′-O-methoxyethyl nucleosides. In some embodiments the modified oligonucleotide comprises at least five contiguous 2′sugar modified nucleosides, independently selected from the group consisting of LNA and 2′-O-methoxyethyl nucleosides.

In some embodiments the modified oligonucleotide comprises at least two contiguous 2′sugar modified nucleosides at the 3′ end of the modified oligonucleotide. In some embodiments the modified oligonucleotide comprises at least three contiguous 2′sugar modified nucleosides at the 3′ end of the modified oligonucleotide. In some embodiments the modified oligonucleotide comprises at least four contiguous 2′sugar modified nucleosides at the 3′ end of the modified oligonucleotide. In some embodiments the modified oligonucleotide comprises at least five contiguous 2′sugar modified nucleosides at the 3′ end of the modified oligonucleotide.

In some embodiments the modified oligonucleotide comprises at least two contiguous 2′sugar modified nucleosides at the 3′ end, independently selected from the group consisting of LNA and 2′-O-methoxyethyl nucleosides. In some embodiments the modified oligonucleotide comprises at least two contiguous LNA nucleosides at the 3′ end. In some embodiments the modified oligonucleotide comprises at least two contiguous 2′-O-methoxyethyl nucleosides at the 3′ end.

In some embodiments the modified oligonucleotide comprises at least three contiguous 2′sugar modified nucleosides at the 3′ end, independently selected from the group consisting of LNA and 2′-O-methoxyethyl nucleosides. In some embodiments the modified oligonucleotide comprises at least three contiguous LNA nucleosides at the 3′ end. In some embodiments the modified oligonucleotide comprises at least three contiguous 2′-O-methoxyethyl nucleosides at the 3′ end. In some embodiments the modified oligonucleotide comprises at least four contiguous 2′ sugar modified nucleosides at the 3′ end, independently selected from the group consisting of LNA and 2′-O-methoxyethyl nucleosides.

In some embodiments, the modified oligonucleotide comprises at least one or more sugar-modified nucleosides, such as one or more LNA nucleosides, and further comprises modified internucleoside linkages, such as phosphorothioate internucleoside linkages. In some embodiments, the modified oligonucleotide comprises at least one or more 2′ substituted nucleosides, such as 2′-O-methoxyethyl nucleosides, and further comprises modified internucleoside linkages, such as phosphorothioate internucleoside linkages.

In some embodiments the modified oligonucleotide comprises a LNA nucleoside at the 3′ most position, or a 2′ substituted nucleoside, such as 2′-methoxyethyl or 2-O-methyl, at the 3′ most position; and may further comprise phosphorothioate internucleoside linkages. Suitable, the modified oligonucleotide may, for example be between 7 and 50 contiguous nucleotides in length, such as 7-30 contiguous nucleotides in length, such as 10-24 contiguous nucleotides in length, such as 12-20 contiguous nucleotides length.

Backbone Modified Oligonucleotides

A backbone modified oligonucleotide is an oligonucleotide which comprises at least one internucleoside linkage other than phosphodiester. The modified oligonucleotide advantageously is a backbone modified oligonucleotide, such as is a phosphorothioate oligonucleotide. In some embodiments the modified oligonucleotide is a phosphorothioate oligonucleotide wherein at least 70% of the internucleoside linkages between the nucleosides of the modified oligonucleotide are phosphorothioate internucleoside linkages, such as at least 80%, such as at least 90% such as all of the internucleoside linkages are phosphorothioate internucleoside linkages.

Sugar Modified Oligonucleotide

A sugar modified oligonucleotide is an oligonucleotide which comprises at least one nucleoside wherein the ribose sugar is replaced with a moiety other than deoxyribose (DNA nucleoside) or ribose (RNA nucleoside). Sugar modified oligonucleotides include nucleosides where the 2′ carbon is substituted with a substituent group other than hydrogen or hydroxyl, as well as bicyclic nucleosides (LNA). In some embodiments the sugar modification is other than 2′fluoro RNA.

2′ Sugar Modified Nucleosides

A 2′ sugar modified nucleoside is a nucleoside which has a substituent other than H or —OH at the 2′ position (2′ substituted nucleoside) or comprises a 2′ linked biradicle capable of forming a bridge between the 2′ carbon and a second carbon in the ribose ring, such as LNA (2′-4′ biradicle bridged) nucleosides.

Indeed, much focus has been spent on developing 2′ sugar substituted nucleosides, and numerous 2′ substituted nucleosides have been found to have beneficial properties when incorporated into oligonucleotides. For example, the 2′ modified sugar may provide enhanced binding affinity and/or increased nuclease resistance to the oligonucleotide. Examples of 2′ substituted modified nucleosides are 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, unlocked nucleic acid (UNA), and 2′-F-ANA nucleoside. For further examples, please see e.g. Freier & Altmann; Nucl. Acid Res., 1997, 25, 4429-4443 and Uhlmann; Curr. Opinion in Drug Development, 2000, 3(2), 293-213, and Deleavey and Damha, Chemistry and Biology 2012, 19, 937. Below are illustrations of some 2′ substituted modified nucleosides.

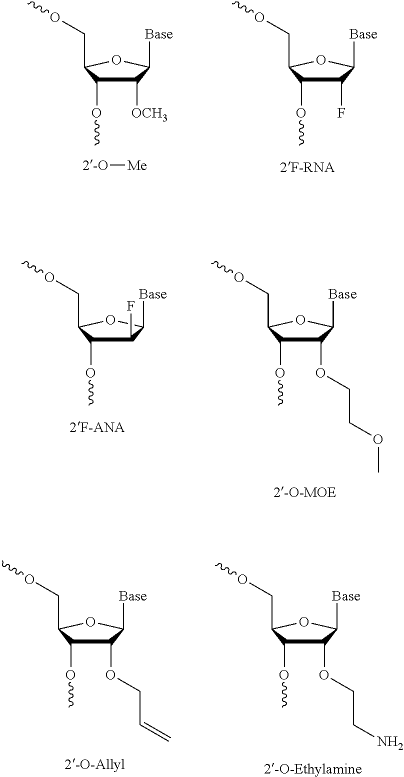

In relation to the present invention 2′ substituted sugar modified nucleosides does not include 2′ bridged nucleosides like LNA.

In some embodiments the modified oligonucleotide does not comprise 2′fluoro modified nucleotides. In some embodiments the modified oligonucleotide comprises at least 2 contiguous modified nucleotises independently selected from the group consisting of 2′-O-alkyl-RNA, 2′-O-2′-alkoxy-RNA, 2′-O-methoxyethyl-RNA (MOE), 2′-amino-DNA, and LNA nucleosides—these are modified nucleosides which comprise a bulky side group at the 2′ position.

Locked Nucleic Acids (LNA)

A “LNA nucleoside” is a 2′-modified nucleoside which comprises a biradical linking the C2′ and C4′ of the ribose sugar ring of said nucleoside (also referred to as a “2′-4′ bridge”), which restricts or locks the conformation of the ribose ring. These nucleosides are also termed bridged nucleic acid or bicyclic nucleic acid (BNA) in the literature. The locking of the conformation of the ribose is associated with an enhanced affinity of hybridization (duplex stabilization) when the LNA is incorporated into an oligonucleotide for a complementary RNA or DNA molecule. This can be routinely determined by measuring the melting temperature of the oligonucleotide/complement duplex.

Non limiting, exemplary LNA nucleosides are disclosed in WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med.Chem. Lett. 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

Further non limiting, exemplary LNA nucleosides are disclosed in Scheme 1.

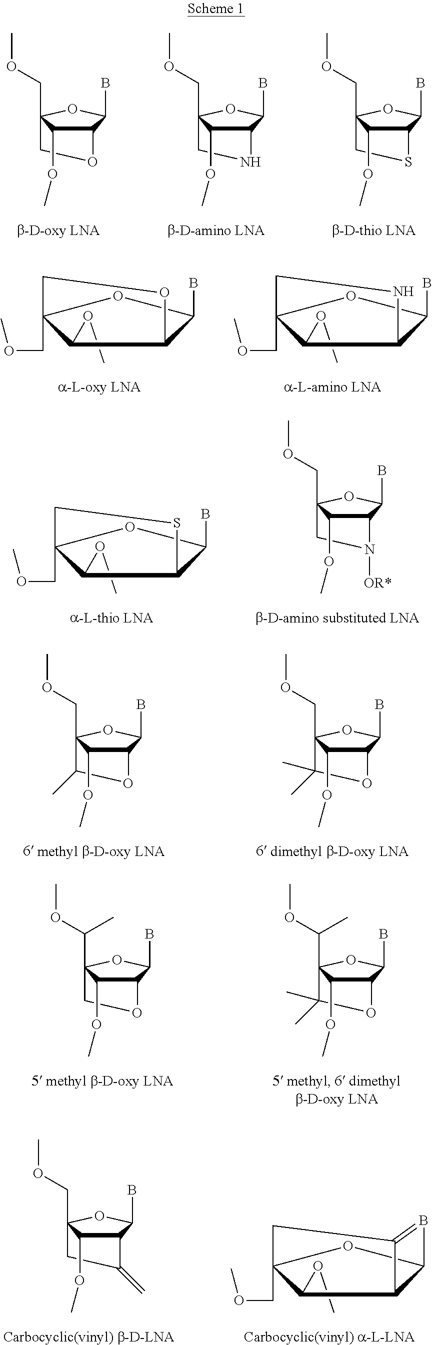

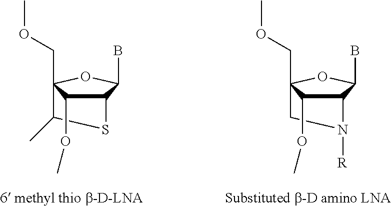

Particular LNA nucleosides are beta-D-oxy-LNA, 6′-methyl-beta-D-oxy LNA such as (S)-6′-methyl-beta-D-oxy-LNA (ScET) and ENA.

A particularly advantageous LNA is beta-D-oxy-LNA.

2′ Substituted Oligonucleotides

In some embodiments the nucleoside modified oligonucleotide comprises at least one 2′ substituted nucleoside, such as at least one 3′ terminal 2′ substituted nucleoside. In some embodiments the 2′ substituted oligonucleotide is a gapmer oligonucleotide, a mixmer oligonucleotide or a totalmer oligonucleotide. In some embodiments the 2′ substitution is selected from the group consisting of 2′methoxyethyl (2′-O-M0E) or 2′O-methyl. In some embodiments, the 3′ nucleotide of the nucleoside modified oligonucleotide is a 2′ substituted nucleoside such as 2′-O-MOE or 2′-O-methyl. In some embodiments the oligonucleotide does not comprise more than four consecutive nucleoside modified nucleosides. In some embodiments the oligonucleotide does not comprise more than three consecutive nucleoside modified nucleosides nucleosides. In some embodiments the oligonucleotide comprises 2 2′-O-MOE modified nucleotides at the 3′ terminal. In some embodiments the nucleoside modified oligonucleotide comprises phosphorothioate internucleoside linkages, and in some embodiments at least 75% of the internucleoside linkages present in the oligonucleotide are phosphorothioate internucleoside linkages. In some embodiments all of the internucleoside linkages of the modified nucleoside oligonucleotide are phosphorothioate internucleoside linkages. Phosphorotioate linked oligonucleotides are widely used for in vivo application in mammals, including their use as therapeutics.

In some embodiments the sugar modified oligonucleotide has a length of 7-30 nucleotides, such as 8-25 nucleotides. In some embodiments the length of the sugar modified oligonucleotide is 10-20 nucleotides, such as 12-18 nucleotides.

Nucleoside oligonucelotides may optionally be conjugated, e.g. with a GalNaC conjugate. If they are conjugated then it is preferable that the conjugate group is positioned other than at the 3′ position of the oligonucleotide, for example the conjugation may be at the 5′ terminal.

LNA Oligonucleotide

In some embodiments the nucleoside modified oligonucleotide comprises at least one LNA nucleoside, such as at least one 3′ terminal LNA nucleoside. In some embodiments the LNA oligonucleotide is a gapmer oligonucleotide, a mixmer oligonucleotide or a totalmer oligonucleotide. In some embodiments the LNA oligonucleotide does not comprise more than four consecutive LNA nucleosides. In some embodiments the LNA oligonucleotide does not comprise more than three consecutive LNA nucleosides. In some embodiments the LNA oligonucleotide comprises 2 LNA nucleotides at the 3′ terminal.

Gapmer

The nucleoside modified oligonucleotide may, in some embodiments be a gapmer oligonucleotide.

The term gapmer as used herein refers to an antisense oligonucleotide which comprises a region of RNase H recruiting oligonucleotides (gap—‘G’) which is flanked 5′ and 3′ by flanking regions (‘F’) which comprise one or more nucleoside modified nucleotides, such as affinity enhancing modified nucleosides (in the flanks or wings). Gapmers are typically 12-26 nucleotides in length and may, in some embodiments comprise a central region (G) of 6-14 DNA nucleosides, flanked either side by flanking regions F which comprises at least one nucleoside modified nucleotide such as 1-6 nucleoside modified nucleosides (F₁₋₆ G₆-14 F1-6). The nucleoside in each flank positioned adjacent to the gap region (e.g. DNA nucleoside region) is a nucleoside modified nucleotide, such as an LNA or 2′-O-MOE nucleoside. In some embodiments all the nucleosides in the flanking regions are nucleoside modified nucleosides, such as LNA and/or 2′-O-MOE nucleosides, however the flanks may comprise DNA nucleosides in addition to the nucleoside modified nucleosides, which, in some embodiments are not the terminal nucleosides.

In some embodiments all the nucleoside in the flanking regions are 2′-O-methoxyethyl nucleosides (a MOE gapmer).

LNA Gapmer

The term LNA gapmer is a gapmer oligonucleotide wherein at least one of the affinity enhancing modified nucleosides in the flanks is an LNA nucleoside. In some embodiments, the nucleoside modified oligonucleotide is a LNA gapmer wherein the 3′ terminal nucleoside of the oligonucleotide is a LNA nucleoside. In some embodiments the 2 3′ most nucleosides of the oligonucleotide are LNA nucleosides. In some embodiments, both the 5′ and 3′ flanks of the LNA gapmer comprise LNA nucleosides, and in some embodiments the nucleoside modified oligonucleotide is a LNA oligonucleotide, such as a gapmer oligonucleotide, wherein all the nucleosides of the oligonucleotide are either LNA or DNA nucleosides.

Mixed Wing Gapmer

The term mixed wing gapmer or mixed flank gapmer refers to a LNA gapmer wherein at least one of the flank regions comprise at least one LNA nucleoside and at least one non-LNA modified nucleoside, such as at least one 2′ substituted modified nucleoside, such as, for example, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA and 2′-F-ANA nucleoside(s). In some embodiments the mixed wing gapmer has one flank which comprises only LNA nucleosides (e.g. 5′ or 3′) and the other flank (3′ or 5′ respectfully) comprises 2′ substituted modified nucleoside(s) and optionally LNA nucleosides. In some embodiments the mixed wing gapmer comprises LNA and 2′-O-MOE nucleosides in the flanks.

Mixmers

A mixmer is an oligonucleotide which comprises both nucleoside modified nucleosides and DNA nucleosides, wherein the oligonucleotides does not comprise more than 4 consecutive DNA nucleosides. Mixmer oligonucleotides are often used for non RNAseH mediated modulation of a nucleic acid target, for example for inhibition of a microRNA or for splice switching modulation or pre-mRNAs.

Totalmer

A totalmer is a nucleoside modified oligonucleotide wherein all the nucleosides present in the oligonucleotide are nucleoside modified. The totalmer may comprise of only one type of nucleoside modification, for example may be a full 2′-O-MOE or fully 2′-O-methyl modified oligonucleotide, or a fully LNA modified oligonucleotide, or may comprise a mixture of different nucleoside modifications, for example a mixture of LNA and 2′-O-MOE nucleosides. In some embodiments the totalmer may comprise one or two 3′ terminal LNA nucleosides.

Tinys

A tiny oligonucleotide is an oligonucleotide 7-10 nucleotides in length wherein each of the nucleosides within the oligonucleotide is an LNA nucleoside. Tiny oligonucelotides are known to be particularly effective designs for targeting microRNAs.

Stereodefined Oligonucleotide

In some embodiments, the modified oligonucleotide is a stereodefined oligonucleotide. A stereodefined oligonucleotide is an oligonucleotide wherein at least one of the internucleoside linkages is a stereodefined internucleoside linkage.

A stereodefined phosphorothioate oligonucleotide is an oligonucleotide wherein at least one of the internucleoside linkages is a stereodefined phosphorothioate internucleoside linkage.

RNAi and siRNA

In some embodiments, the modified oligonucleotide may be an RNAi molecule such as an siRNA or an siRNA sense and/or antisense strand. Herein, the term “RNA interference (RNAi) molecule” refers to any molecule inhibiting RNA expression or translation via the RNA reducing silencing complex (RISC). A small interfering RNA (siRNA) is typically a double-stranded RNA complex comprising a sense and an antisense oligonucleotide, which when administered to a cell, results in the incorporation of the antisense strand into the RISC complex (siRISC) resulting in the RISC associated inhibition of translation or degradation of complementary RNA target nucleic acids in the cell. The sense strand is also referred to as the passenger strand, and the antisense strand as the guide strand. A small hairpin RNA (shRNA) is a single nucleic acid molecule which forms a hairpin structure that is able to degrade mRNA via RISC. RNAi nucleic acid molecules may be synthesized chemically (typical for siRNA complexes) or by in vitro transcription, or expressed from a vector. Typically, the antisense strand of an siRNA (or antisense region of a shRNA) is 17-25 nucleotide in length, such as 19-23 nucleotides in length. In an siRNA complex, the antisense strand and sense strand form a double stranded duplex, which may comprise 3′ terminal overhangs of e.g. 1-3 nucleotides, or may be blunt ended (no overhang at one or both ends of the duplex).

It will be recognized that RNAi may be mediated by longer dsRNA substrates which are processed into siRNAs within the cell (a process which is thought to involve the dsRNA endonuclease DICER). Effective extended forms of Dicer substrates have been described in U.S. Pat. Nos. 8,349,809 and 8,513,207, hereby incorporated by reference.

RNAi agents may be chemically modified using modified internucleotide linkages and high affinity nucleosides, such as 2′-4′ bicyclic ribose modified nucleosides, including LNA and cET. See for example WO 2002/044321 which discloses 2′O-Methyl modified siRNAs, WO2004083430 which discloses the use of LNA nucleosides in siRNA complexes, known as siLNAs, and WO2007107162 which discloses the use of discontinuous passenger strands in siRNA such as siLNA complexes. WO03006477 discloses siRNA and shRNA (also referred to as stRNA) oligonucleotide mediators of RNAi. Harborth et al., Antisense Nucleic Acid Drug Dev. 2003 April; 13(2):83-105 refers to the sequence, chemical, and structural variation of small interfering RNAs and short hairpin RNAs and the effect on mammalian gene silencing.

It will be recognized that the methods of the present invention enable the simultaneous sequencing of both strands of a siRNA complex.

Antisense Oligonucleotides

In some embodiments the modified oligonucleotide is an antisense oligonucleotide.
The term “Antisense oligonucleotide” as used herein is defined as oligonucleotides capable of modulating expression of a target gene by hybridizing to a target nucleic acid, in particular to a contiguous sequence on a target nucleic acid. The antisense oligonucleotides are not essentially double stranded and are therefore not siRNAs or shRNAs. An antisense oligonucleotides is single stranded. It is understood that single stranded oligonucleotides can form hairpins or intermolecular duplex structures (duplex between two molecules of the same oligonucleotide), as long as the degree of intra or inter self-complementarity is less than 50% across of the full length of the oligonucleotide.

In some embodiments the antisense oligonucleotide is a sugar modified oligonucleotide.

Nucleotides

Nucleotides are the building blocks of oligonucleotides and polynucleotides, and for the purposes of the present invention include both naturally occurring and non-naturally occurring nucleotides. In nature, nucleotides, such as DNA and RNA nucleotides comprise a ribose sugar moiety, a nucleobase moiety and one or more phosphate groups (which is absent in nucleosides). Nucleosides and nucleotides may also interchangeably be referred to as “units” or “monomers”.

Modified Nucleoside

The term “modified nucleoside” or “nucleoside modification” as used herein refers to nucleosides modified as compared to the equivalent DNA or RNA nucleoside by the introduction of one or more modifications of the sugar moiety or the (nucleo)base moiety. In a preferred embodiment the modified nucleoside comprise a modified sugar moiety. The term modified nucleoside may also be used herein interchangeably with the term “nucleoside analogue” or modified “units” or modified “monomers”. Nucleosides with an unmodified DNA or RNA sugar moiety are termed DNA or RNA nucleosides herein. Nucleosides with modifications in the base region of the DNA or RNA nucleoside are still generally termed DNA or RNA if they allow Watson Crick base pairing.

Modified Internucleoside Linkages

The term “modified internucleoside linkage” is defined as generally understood by the skilled person as linkages other than phosphodiester (PO) linkages, that covalently couples two nucleosides together. The oligonucleotides of the invention may therefore comprise modified internucleoside linkages. In some embodiments, the modified internucleoside linkage increases the nuclease resistance of the oligonucleotide compared to a phosphodiester linkage. For naturally occurring oligonucleotides, the internucleoside linkage includes phosphate groups creating a phosphodiester bond between adjacent nucleosides. Modified internucleoside linkages are particularly useful in stabilizing oligonucleotides for in vivo use, and may serve to protect against nuclease cleavage at regions of DNA or RNA nucleosides in the oligonucleotide of the invention, for example within the gap region of a gapmer oligonucleotide, as well as in regions of modified nucleosides, such as region F and F′.

In an embodiment, the oligonucleotide comprises one or more internucleoside linkages modified from the natural phosphodiester, such one or more modified internucleoside linkages that is for example more resistant to nuclease attack. Nuclease resistance may be determined by incubating the oligonucleotide in blood serum or by using a nuclease resistance assay (e.g. snake venom phosphodiesterase (SVPD)), both are well known in the art. Internucleoside linkages which are capable of enhancing the nuclease resistance of an oligonucleotide are referred to as nuclease resistant internucleoside linkages. In some embodiments at least 50% of the internucleoside linkages in the oligonucleotide, or contiguous nucleotide sequence thereof, are modified, such as at least 60%, such as at least 70%, such as at least 80 or such as at least 90% of the internucleoside linkages in the oligonucleotide, or contiguous nucleotide sequence thereof, are nuclease resistant internucleoside linkages. In some embodiments all of the internucleoside linkages of the oligonucleotide, or contiguous nucleotide sequence thereof, are nuclease resistant internucleoside linkages. It will be recognized that, in some embodiments the nucleosides which link the oligonucleotide of the invention to a non-nucleotide functional group, such as a conjugate, may be phosphodiester.

A preferred modified internucleoside linkage is phosphorothioate.

Phosphorothioate internucleoside linkages are particularly useful due to nuclease resistance, beneficial pharmacokinetics and ease of manufacture. In some embodiments at least 50% of the internucleoside linkages in the oligonucleotide, or contiguous nucleotide sequence thereof, are phosphorothioate, such as at least 60%, such as at least 70%, such as at least 80% or such as at least 90% of the internucleoside linkages in the oligonucleotide, or contiguous nucleotide sequence thereof, are phosphorothioate. In some embodiments all of the internucleoside linkages of the oligonucleotide, or contiguous nucleotide sequence thereof, are phosphorothioate.

Nuclease resistant linkages, such as phosphorothioate linkages, are particularly useful in oligonucleotide regions capable of recruiting nuclease when forming a duplex with the target nucleic acid, such as region G for gapmers. Phosphorothioate linkages may, however, also be useful in non-nuclease recruiting regions and/or affinity enhancing regions such as regions F and F′ for gapmers. Gapmer oligonucleotides may, in some embodiments comprise one or more phosphodiester linkages in region F or F′, or both region F and F′, which the internucleoside linkage in region G may be fully phosphorothioate.

Advantageously, all the internucleoside linkages in the contiguous nucleotide sequence of the oligonucleotide are phosphorothioate linkages.

It is recognized that, as disclosed in EP2 742 135, antisense oligonucleotide may comprise other internucleoside linkages (other than phosphodiester and phosphorothioate), for example alkyl phosphonate/methyl phosphonate internucleosides, which according to EP2 742 135 may for example be tolerated in an otherwise DNA phosphorothioate the gap region.

Nucleobase

The term nucleobase includes the purine (e.g. adenine and guanine) and pyrimidine (e.g. uracil, thymine and cytosine) moiety present in nucleosides and nucleotides which form hydrogen bonds in nucleic acid hybridization. In the context of the present invention the term nucleobase also encompasses modified nucleobases which may differ from naturally occurring nucleobases, but are functional during nucleic acid hybridization. In this context “nucleobase” refers to both naturally occurring nucleobases such as adenine, guanine, cytosine, thymidine, uracil, xanthine and hypoxanthine, as well as non-naturally occurring variants. Such variants are for example described in Hirao et al (2012) Accounts of Chemical Research vol 45 page 2055 and Bergstrom (2009) Current Protocols in Nucleic Acid Chemistry Suppl. 37 1.4.1.

In a some embodiments the nucleobase moiety is modified by changing the purine or pyrimidine into a modified purine or pyrimidine, such as substituted purine or substituted pyrimidine, such as a nucleobased selected from isocytosine, pseudoisocytosine, 5-methyl cytosine, 5-thiozolo-cytosine, 5-propynyl-cytosine, 5-propynyl-uracil, 5-bromouracil 5-thiazolo-uracil, 2-thio-uracil, 2′thio-thymine, inosine, diaminopurine, 6-aminopurine, 2-aminopurine, 2,6-diaminopurine and 2-chloro-6-aminopurine.

The nucleobase moieties may be indicated by the letter code for each corresponding nucleobase, e.g. A, T, G, C or U, wherein each letter may optionally include modified nucleobases of equivalent function. For example, in the exemplified oligonucleotides, the nucleobase moieties are selected from A, T, G, C, and 5-methyl cytosine. Optionally, for LNA gapmers, 5-methyl cytosine LNA nucleosides may be used.

Nucleobase Sequence

A nucleobase sequence refers to the sequence of nucleobases present in a oligonucleotide or polynucleotide. The nucleobase sequence of an oligonucleotide usually refers to the sequence of A, T, C and G nucleobases. The presence of a 5-methyl cytosine base within an oligonucleotide may therefore be identified as a cytosine residue in a nucleobase sequence identified by a sequencing method. Likewise, a uracil nucleobase may be identified as a tyrosine base in a sequencing method.

Nucleic Acid Sequence

The term “nucleic acid sequence” refers to a nucleic acid molecule which comprises a contiguous sequence of nucleotides, and may comprise the sequence of nucleotides present in the modified oligonucleotide, or the reverse complement thereof.

Complementarity

The term “complementarity” describes the capacity for Watson-Crick base-pairing of nucleosides/nucleotides. Watson-Crick base pairs are guanine (G)-cytosine (C) and adenine (A)−thymine (T)/uracil (U). It will be understood that oligonucleotides may comprise nucleosides with modified nucleobases, for example 5-methyl cytosine is often used in place of cytosine, and as such the term complementarity encompasses Watson Crick base-paring between non-modified and modified nucleobases (see for example Hirao et al (2012) Accounts of Chemical Research vol 45 page 2055 and Bergstrom (2009) Current Protocols in Nucleic Acid Chemistry Suppl. 37 1.4.1).

Identity (Nucleotide Sequences)

The term “Identity” as used herein, refers to the proportion of nucleotides (expressed in percent) of a contiguous nucleotide sequence in a nucleic acid molecule (e.g. oligonucleotide) which across the contiguous nucleotide sequence, are identical to a reference sequence (e.g. a sequence motif). The percentage of identity is thus calculated by counting the number of aligned bases that are identical (a match) between two sequences (in the contiguous nucleotide sequence of the compound of the invention and in the reference sequence), dividing that number by the total number of nucleotides in the oligonucleotide and multiplying by 100. Therefore, Percentage of Identity=(Matches×100)/Length of aligned region (e.g. the contiguous nucleotide sequence). Insertions and deletions are not allowed in the calculation the percentage of identity of a contiguous nucleotide sequence. It will be understood that in determining identity, chemical modifications of the nucleobases are disregarded as long as the functional capacity of the nucleobase to form Watson Crick base pairing is retained (e.g. 5-methyl cytosine is considered identical to a cytosine for the purpose of calculating % identity).

Hybridization

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex.

The term “hybridizing” or “hybridizes” as used herein is to be understood as two nucleic acid strands (e.g. an oligonucleotide and a target nucleic acid) forming hydrogen bonds between base pairs on opposite strands thereby forming a duplex. The affinity of the binding between two nucleic acid strands is the strength of the hybridization. It is often described in terms of the melting temperature (Tm) defined as the temperature at which half of the oligonucleotides are duplexed with the target nucleic acid.

Identity (Amino Acid Sequences)

Identity: The relatedness between two amino acids is described by the parameter “identity”. For purposes of the present invention, the degree of identity between two amino acid sequences is determined using the Needleman-Wunsch algorithm (Needleman and Wunsch, 1970, J. Mo/. Biol. 5 48: 443-453) as implemented in the Needle program of the EMBOSS package (EMBOSS: The European Molecular Biology Open Software Suite, Rice et al., 2000, Trends in Genetics 16: 276-277; http://emboss.org), preferably version 3.0.0 or later. The optional parameters used are gapopen penalty of 10, gap extension penalty of 0.5, and the EBLOSUM62 (EMBOSS version of BLOSUM62) substitution matrix. The output of Needle labeled “longest identity” (obtained using the 10-nobrief option) is used as the percent identity and is calculated as follows: (Identical Residues×100)/(Length of Alignment−Total Number of Gaps in Alignment).

Aptamer

Aptamers are oligonucleotides, typically 20-60 nucleotide in length which bind to a specific target molecule through a non-nucleic acid hybridization mechanism. The modified oligonucleotide of the invention may be an aptamer, or may comprise a region of nucleic acid sequence which functions as an aptamer. In some embodiments the modified oligonucleotide comprises at least two, such as at least three contiguous 2′sugar modified nucleosides, independently selected from the group consisting of LNA and 2′-O-methoxyethyl nucleosides. In some embodiments the modified oligonucleotide comprises at least two, such as at least three contiguous 2′ sugar modified nucleosides located at the 3′ end of the modified oligonucleotide, such as 2′sugar modified nucleotides independently selected from the group consisting of LNA and 2′-O-methoxyethyl nucleosides. In some embodiments the modified oligonucleotide comprises at least two, such as at least three contiguous LNA nucleosides located at the 3′ end of the modified oligonucleotide.

Ligating

Ligation refers to the covalent linking of two nucleic acid fragments, such as oligonucleotides, Ligation typically involves the formation of a phosphate bond between a 3′-OH group on one nucleic acid fragment with the 5′ phosphoryl group on another nucleic acid fragment, and may be catalyzed by a ligase enzyme, such as T4 DNA ligase.

The ligation of the 3′ capture probe to the modified oligonucleotide may in some embodiments use T4DNA ligase. Suitably the 5′ most nucleoside(s) of the 3′capture probe may be DNA.

The ligation of the 5′ capture probe to the modified oligonucleotide may in some embodiments use T4 RNA ligase, or T4 RNA ligasell. Suitably the 3′ most nucleoside(s) of the 5′capture probe may be RNA.

The 3′ Capture Probe Oligonucleotide

As described herein, the 3′ capture probe oligonucleotide, is an oligonucleotide which comprises a first primer binding site and which, in the methods of the invention, is ligated to the 3′terminus of the modified oligonucleotide, thereby enabling polymerase based chain elongation using the modified oligonucleotide as a template. The first primer binding site may also be used as a binding site for sequencing primers, such as solid phase bound primers.

The 3′ capture probe may comprise a 3′ region which is complementary to the 3′ region of the modified oligonucleotide, (or is a degenerate region), thereby capturing the 3′ region of the oligonucleotide facilitating ligation of the capture probe to the modified oligonucleotide. Alternatively a splint ligation may be performed.

In some embodiments, for primer based sequencing, the 3′ capture probe oligonucleotide may further comprise a sequencing primer binding site, such as solid phase bound primer. The capture probe may therefore comprise a binding site for binding to a solid support used in massively parallel sequencing, such as a flow cell binding site, which may be common to the first primer side or may be an independent region which is separate from or overlapping with the first primer binding site. It will be understood that when the sequencing primer binding site is different from the first primer binding site, the sequencing primer binding site is upstream (i.e. 5′ of the first primer binding site), thereby insuring the incorporation of the sequencing primer binding site in the first strand synthesis from the capture probe.

In some embodiments the capture probe of the invention comprises a cleavable linkage group, e.g. for use in self-priming 3′ capture probe oligonucleotides. A self-priming 3′ capture probe may be used to initiate 5′-3′ chain elongation (first strand synthesis) without the addition of a first primer by virtue of two regions of self-complementarity between two regions within the capture probe forming a duplex (may be referred to herein as a second duplex region), wherein the self-priming capture probe comprises a cleavable linkage which when cleaved provides a substrate for 5′-3′ polymerase mediated chain elongation (e.g. a duplex region comprising a 3′terminal —OH group). The cleavable linkage may be positioned adjacent to the 3′ most region of the self-complementary region. The cleavable linkage may be any cleavable group, for example may be UV cleavable or enzymatically cleaved. One preferred cleavage group is a region comprising a mismatched RNA nucleoside, which can be cleaved using a RNaseH2 enzyme. For efficient RNaseH2 cleavage the mismatched RNA nucleoside(s) may be flanked by 3 or 4 3′ (and optionally 5′) nucleosides which form part of the capture probe duplex formed between the two distal regions.

In a preferable embodiment, the 3′ capture probe is an oligonucleotide comprises at least one 5′ DNA nucleoside which is used to “capture” the nucleoside modified oligonucleotide via ligation (e.g. using T4 DNA ligase, other ligation methods may be used). The capture may occur by the ligation of the 5′ end of the capture probe to the 3′ nucleotide of the modified nucleoside oligonucleotide. In some embodiments, it may be advantageous that the capture probe further comprises a region which is complementary to a region on target modified oligonucleotide sequence which is used to capture the target nucleic acid sequence via nucleic acid hybridization (Watson-Crick base pairing) prior to the ligation step. The use of hybridization between a region of the capture probe and a complementary region on the modified oligonucleotide effectively enriches the local substrate concentration, enhancing the efficacy of the ligation step. PCT/EP2017/078695 discloses capture probes which may be used in the methods of the invention. The 3′ capture probe may further comprise a PCR primer binding site for use in an amplification step (PCT step) where including in the method of the invention.

The invention provides or uses a 3′ capture probe oligonucleotide, for use in parallel sequencing of a sugar modified oligonucleotide, comprising 5′-3′:
A. A 5′ region comprising at least 3 contiguous nucleotides of predetermined sequence, wherein the 5′ most nucleotide is a nucleotide with a terminal 5′ phosphate group.
B. Optionally a parallel sequencing reaction bar code region comprising a region of predetermined nucleotide sequence,
C. optionally a region of degenerate or predetermined nucleotides, positioned 3′ of region B, or 5′ to region B
D. a solid phase sequencing primer binding site
E. optionally a first primer binding site
F. optionally a linker region (may be a sequence of nucleotides)
G. a contiguous sequence of nucleotides which are complementary to the predetermined sequence A of the first segment (a first duplex region)
H. a region of at least 2 nucleotides, wherein the 3′ most nucleotide is a terminal nucleotide with a blocked 3′ terminal group.

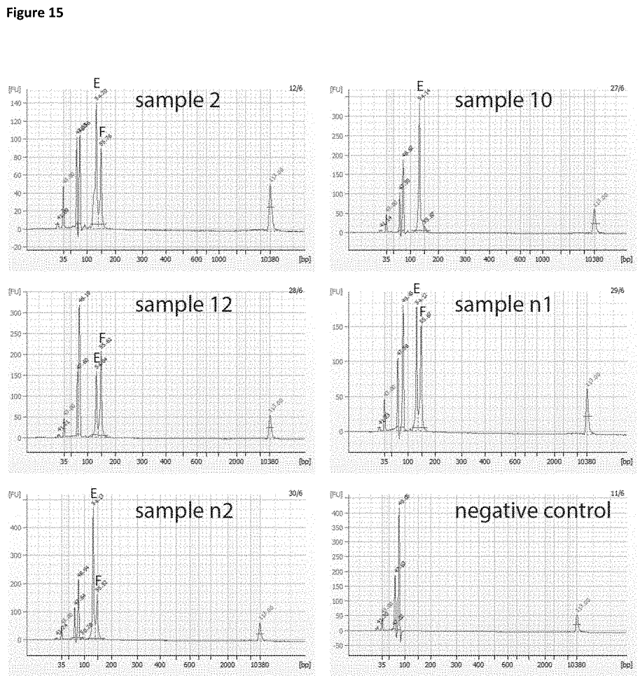

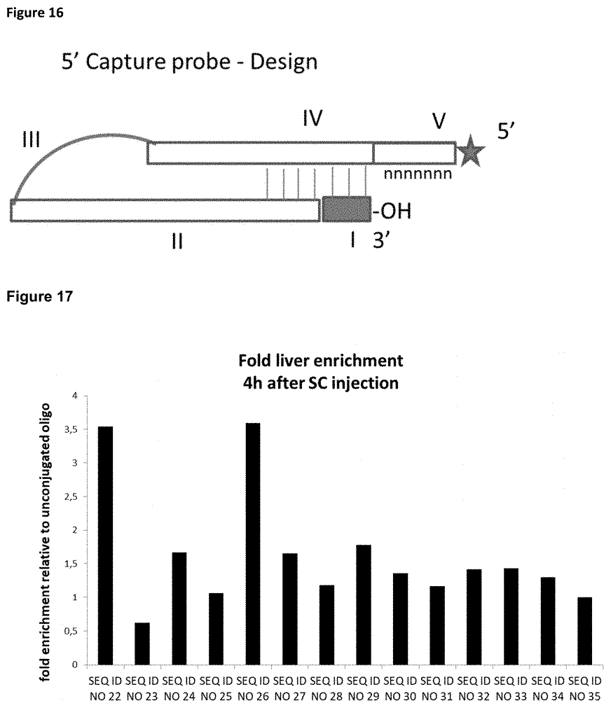

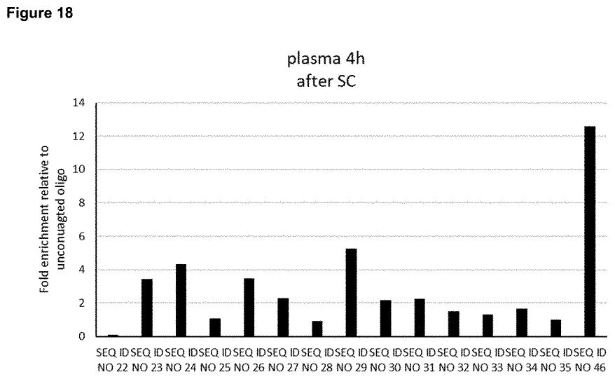

See FIG. 7 for a diagrammatic representation. In the embodiment where the capture probe does not comprise a first primer binding site, the first primer may be designed to hybridise to region D (i.e. region D may be both a sequencing primer binding site and used as a first primer binding site).

The invention provides or uses a 3′ capture probe oligonucleotide, for use in parallel sequencing of a sugar modified oligonucleotide, comprising 5′-3′:
A. A 5′ region comprising at least 3 contiguous nucleotides of predetermined sequence, wherein the 5′ most nucleotide is a nucleotide with a terminal 5′ phosphate group.
B. Optionally a parallel sequencing reaction bar code region comprising a region of predetermined nucleotide sequence,
C. optionally a region of degenerate or predetermined nucleotides, positioned 3′ of region B, or 5′ to region B
D. a solid phase sequencing primer binding site
E. optionally a first primer binding site
F. optionally a linker region (may be a sequence of nucleotides)
F′ a region which forms a duplex with the first primer binding site and/or solid phase sequencing primer binding site, wherein the duplex comprises a cleavable linker,
G. a contiguous sequence of nucleotides which are complementary to the predetermined sequence A of the first segment (a first duplex region)
H. a region of at least 2 nucleotides, wherein the 3′ most nucleotide is a terminal nucleotide with a blocked 3′ terminal group.

The cleavage of the cleavable linker in region F′ leaves a 3′ terminus which can be used for first strand synthesis without the use of a exogenously added first primer (i.e. forms a self priming capture probe).

See FIG. 7 as an example of an exemplary 3′ capture probes which may be used in the method of the invention:

Region A

In some embodiments, region A comprises or consists of at least 3 contiguous nucleotides, of predetermined sequence, wherein the 5′ terminal nucleotide is a DNA nucleotide which comprises a 5′ phosphate group. The at least 3 contiguous nucleotides are complementary to and can hybridize to region G (the first duplex region). In some embodiments the at least 3 contiguous nucleotides of region A are DNA nucleotides.

In some embodiments, region A comprises or consists of at least 3 contiguous nucleotides, such as 3-10 contiguous nucleotides, such as 3-10 DNA nucleotides.

Region B

Region B may be used as or is a parallel sequencing “reaction bar code” region comprising a region of predetermined nucleotide sequence, such as a region of 3-20 nucleotides, such as DNA nucleotides. It is advantageous that the capture probe comprises region B as it allows for the pooling of samples from separate capture probe ligations to be pooled prior to sequencing in a common parallel sequencing run. The use of different capture probes with distinct region B sequences thereby allows the post sequencing separation of sequence data from the separate capture probe ligations.

Region C

Region C is an optional sequence of nucleotides positioned 3′ of region A which may comprise a predetermined sequence or a degenerate sequence, or in some embodiments both a predetermined sequence part and a degenerate sequence part. The length of region C, when present may be modulated according to use. When a degenerate sequence is used it may allow the “molecular bar coding” of amplification products in subsequent sequencing steps, allowing for the determination of whether a particular amplification product is unique. This allows for comparative quantification of different oligonucleotides present in a heterogenous mixture of oligonucleotides. In some embodiments region C comprises 3-30 degenerate contiguous nucleotides, such as 3-30 degenerate contiguous DNA nucleotides. In some embodiments region C comprises universal nucleotides, such as inosine nucleotides.

It is known that some sequences may be preferentially amplified during PCR, and as such by counting the occurrence of a genetic “barcode sequence”, originating from the degenerate sequence, you can determine the pre-amplification relative quantities (see e.g. Kielpinski & Vinter, NAR (2014) 42 (8): e70.

In some embodiments region C introduces a semi-degenerate sequence, which allows benefit of both a bar code sequence and a predetermined sequence. Additional benefit is a quality control of the barcode sequence (see e.g. Kielpinski et al., Methods in Enzymology (2015) vol. 558, pages 153-180). A semi-degenerate sequence has a selected semi-degenerate nucleobase at each position (based upon the Need a definition of semi-degenerate—add IUPAC codes, R, Y, S, W, K, M, B, D, H and V (See table 3).

In some embodiments region C has both degenerate sequence and predetermined sequence, or has both degenerate sequence and semi-degenerate sequence, or has both predetermined sequence and semi-degenerate sequence, or has degenerate sequence and predetermined sequence and semi-degenerate sequence.

If region C comprises a predetermined sequence it may for example provide an alternative, or nested, primer site, upstream of the first primer site, the use of nested primer sites is a well-known tool for reducing non-specific binding during PCR amplification. In some embodiments region C comprises 3-30 predetermined contiguous nucleotides, such as 3-30 predetermined contiguous DNA nucleotides.

In some embodiments the capture probe does not comprise region C.

It will be understood that functionally region C may be positioned 5′ to region B or 3′ to region B.

In some embodiments, when present regions C consists or comprises at least 3 contiguous degenerate nucleosides, such as 3, 4, 5, 6, 7, 8, 9 or 10 contiguous degenerate nucleosides.

Region D

Region D is a solid phase primer binding site, also referred to as the sequencing primer binding site, which is used to capture the adapter ligation product, or optionally a PCR product prepared from the adapter ligation product, to a oligonucleotide attached to a solid phase support prior to an optional clonal amplification, and subsequent parallel sequencing. Region D may also be used as a first primer binding site to initiate first strand synthesis.

In self priming capture probes, region D may form part of the duplex (the second duplex) which hybridizes to a downstream (3′) region (F′) which comprises a cleavable linkage such as a mismatched RNA nucleotide(s), as long as this does not compromise the binding of region D with the primer bound to the solid phase support (i.e. the integrity of the sequencing primer binding site is maintained post cleavage of region F′.).

Region E

Region E is a first primer binding site, which is used to initiate first strand synthesis. Region E may not be necessary to include when region D is used as the first primer binding site. Functionally the first primer binding site region E may therefore be the same as the sold phase primer binding site (D) or may partially overlap with region D.

In self priming capture probes of the invention, region E may form part of the duplex (the second duplex) which hybridizes to a downstream (3′) region (F′) which comprises a cleavable linkage such as a mismatched RNA nucleotide(s).

Region G

Region G is a region of nucleotides which are complementary to region A which form a duplex with region A. It is beneficial if region G does not comprise RNA nucleosides which are complementary to region A, and it is also beneficial that the nucleoside present in region G which is complementary to and hybridizes to the 5′ terminal nucleoside of the capture probe (5′ nucleoside of region A) is a DNA nucleoside. This results in the formation of a DNA/DNA duplex when regions A and G hybridize. In some embodiments the two or three 3′ most nucleosides of region G are DNA nucleosides. In some embodiments all of the nucleosides of region G are DNA nucleosides. In some embodiments, region G comprises at least 3 contiguous nucleotides that are complementary to and can hybridize to region A. In some embodiments the at least 3 contiguous nucleotides of region G are DNA nucleotides.

In some embodiments, region G comprises or consists of 3-10 contiguous nucleotides, such as 3-10 DNA nucleotides. In some embodiments, the nucleotides of region A and region G are DNA nucleotides. The length and composition (e.g. G/C vs NT) of the complementary sequences A and G may be used to modulate the strength of hybridization, allowing for optimization of the capture probe. It is also recognized that introduction of mismatches within a complementary sequence can be used to decrease the hybridization strength (see WO2014110272 for example). In some embodiments region A and G do not form a contiguous complementary sequence, but due to partial complementarity in some embodiments regions A and G form a duplex when admixed with the sample. The 3′ most base pair of regions A and G should be a complementary base pair, and in some embodiments the two or three most base pairs of regions A and G are complementary base pairs. In some embodiments, these 3′ base pair(s) are DNA base pairs.

Region H

Region H serves the purpose of hybridizing the 3′ capture probe oligonucleotide to the nucleoside modified oligonucleotide that is to be detected, captured, sequenced and/quantified.

Region H is a region of at least two or three nucleotides which form a 3′ overhang, when region A and G, of the complementary sequences thereof, are hybridized. The 3′ terminal nucleoside of region H is blocked at the 3′ position (i.e. does not comprise a 3′ —OH group).

In some embodiments, region H has a length of at least 3 nucleotides. The optimal length of region H may depend, at least on the length of the oligonucleotide to be captured, and the present inventors have found that region H can function with an overlap of 2 nucleotides, for example when using an RNase treated sample, and preferably is at least 3 nucleotides.

In some embodiments, region H comprises a degenerate sequence, or a semi-degenerate sequence, which allows for the capture of oligonucleotides without prior knowledge of the oligonucleotide sequence. The capture of oligonucleotides without prior knowledge of their sequence is particularly useful in identifying specific oligonucleotides from a library of different oligonucleotide sequences which have a desired biodistribution, or for the identification of partial oligonucleotide degradation products. The probes and methods of the invention may also be applied to the capture and identification of aptamers.

In some embodiments, region H comprises a predetermined sequence, allowing for the capture of nucleoside modified oligonucleotides with a known sequence. The use of a predetermined capture region H allows for capture, detection and quantification of therapeutic oligonucleotides in vivo, for example for pre-clinical or clinical development or subsequently for determining local tissue or cellular concentration or exposure in patient derived material. The determination of compound concentration in patients can be important in optimizing the dosage of therapeutic oligonucleotides in patients.

In some embodiments, region H comprises a high affinity modified nucleosides, such as one or more LNA nucleosides. Use of high affinity modified nucleosides such as LNA in region H allows for the use of shorter region of nucleotides whilst allowing for efficient capture of the modified oligonucleotide. In this respect for LNA modified oligonucleotides, the LNA/LNA hybrid is particularly strong. It will be understood that by selective use of high affinity modified nucleosides in region H the capture efficacy can be optimsied.

Region H may be a region of predetermined nucleotide sequence or a degenerate (or partially degenerate) sequence. A predetermined nucleotide sequence may be used where the 3′ region of the modified oligonucleotide is known. A degenerate sequence of region H may be used to ligate modified oligonucleotides of unknown sequence or where there may be heterogeneity within the 3′ regions within a population of modified oligonucleotides. In some embodiments, region H consists or comprises at least 4 contiguous degenerate nucleosides, such as 4, 5, 6, 7, 8, 9, 10, 11 or 12 contiguous degenerate nucleosides.

In some embodiments, the nucleosides of regions A, B, C, D, and E when present are DNA nucleosides.

The Linker Moiety (F) (Optional)

Region F

Region F is an optional region and is illustrated by the thin lines joining region E and G in FIG. 7. In the absence of region E it may link region D and region G, or region D or E to region F′. In some embodiments the capture probe oligonucleotide does not comprise a non-nucleosidic linker.

Region F may be used to facilitate for the capture probe regions A and G to hybridize to rom the first duplex region, and may be a region of nucleosides or may comprises a non-nucleotide linker. In some embodiments region F is present and region F comprises at least 3 or 4 nucleotides, such as at least 3 or 4 DNA nucleotides, such as 4-25 nucleotides.

A key function of region F is to allow the duplex formation between regions A and G (the first duplex), and in the self-priming 3′ capture probe embodiment, the formation of the second duplex formation between region F′ and region E, or between region F′ and region D, or between region F′ and overlapping with regions D and E. region F may therefore form a intramolecular hairpin structure within the capture probe. It is however recognized that in some embodiments region F is not required, e.g. when region D (and optionally region B and/or C) are capable of forming the intramolecular hairpin allowing the duplex formation between regions A and G. In the self-priming capture probe embodiment, it is envisaged that region F is advantageous.

The region of nucleotides may or may not comprise a modification which prevents polymerase read through (e.g. an inversed nucleotide linkage).

The advantage of preventing read-through of the DNA polymerase from region D to G, e.g. via a non-nucleotide linker or a polymerase inhibiting modification, is that it prevents the formation of an alternative template molecule. Such alternative template molecules result in mispriming of the primers specific to the nucleoside modified oligonucleotide on the 5′ region of the capture probe.

In some embodiments of the invention the linker moiety F may be a region of nucleotides which allow region A and G to hybridise.

In some embodiments region F comprises a polymerase blocking linker, such as a 06-32 polyethyleneglycol linker, such as a C18 polyethyleneglycol linker or an alkyl linker. Other non-limiting exemplary linker groups which may be used are disclosed in PCT/EP2017/078695.

Splint Ligation

The 3′ and/or 5′ capture probe may in some embodiments be linear capture probes. With linear capture probes it may be advantageous to use a splint ligation primer in conjunction with the linear capture probe: For example, a splint ligation primer may hybridize to the 5′ region of the 3′ capture probe and the 3′ region of the modified oligonucleotide, thereby aligning the ends to be ligated. Alternatively, a splint ligation primer may hybridize to the 3′ region of the 5′ capture probe and the 5′ region of the modified oligonucleotide, thereby aligning the ends to be ligated.

5′ Capture Probe

In some embodiments a 5′ capture probe is ligated to the 5′ end of the modified oligonucleotide prior to first strand synthesis. In order to facilitate 5′ capture probe ligation the 5′ terminus of the modified oligonucleotide should comprise a 5′ phosphate group. For modified oligonucleotides which do not comprise a 5′ phosphate group they may be phosphorylated using T4 polynucleotide kinase.

In some embodiments, prior to the ligation of the 5′ capture probe to the 5′ terminus of the modified oligonucleotide, in the event that the modified oligonucleotide does not comprise a 5′ phosphate group, a further step may be performed comprising the phosphorylation of the 5′ terminus of the modified oligonucleotide. T4 polynucleotide kinase may be used to phosphorylate 5′ ends of oligonucleotides.

The 5′ capture probe may advantageously comprise 1 or more 3′ terminal RNA nucleosides, such as 1-10 RNA nucleotides, such as 2, 3, 4, 5 or 6 RNA nucleotides. Using RNA nucleotide(s) at the 3′ terminus allows the use of T4 RNA ligase or T4 RNA ligase II to catalyse the ligation of the 5′ capture probe to the modified oligonucleotide. The 5′ capture probe comprises a primer binding site which may be used for PCR or primer based sequencing approaches. The 5′ capture probe may further comprise a flow cell primer binding site, and/or a clonal amplification primer binding site and/or a sequencing primer binding sites. It will be understood that flow cell binding site, clonal amplification primer binding site and/or sequencing primer binding site may be incorporated into the PCR primer recognizing the 5′ capture probe PCR primer binding site.

The 5′ capture probe may comprise a bar code sequence which may be positioned 3′ of the primer binding site (ensuring it is retained in down-stream PCR or clonal amplification steps). The bar code sequence may be a molecule bar code or may be a sequence reaction bar code, or the 5′capture probe may comprise both a molecular bar code or sequence reaction bar code.

In order to facilitate positioning of the 3′ terminus of the 5′ capture probe to the 5′ end of the modified oligonucleotide a splint ligation approach may be used. Alternatively, the capture probe may comprise a 5′ region which forms a duplex with a complementary sequence in the 3′region of the 5′capture probe, wherein the 5′ region positions a region of nucleotides which are complementary to the 5′ end of the modified oligonucleotide or a sequence of degenerate oligonucleotides. The 5′ terminus of the 5′ capture probe should be other than 5′ phosphate to avoid self-ligation of the capture probe.

The invention provides for a capture probe oligonucleotide, which may be used in the methods of the invention, comprising 3′-5:
I. a first region comprising a 3′terminal RNA nucleoside or a 3′ terminal region of RNA nucleotides with a 3′ terminal —OH group;
II. a second region of nucleotides which comprises the complement of a primer binding site, e.g. a PCR primer binding site (or a primer site for second strand synthesis);
III. A third region, comprising
   a. A sequence of nucleotides; or
   b. a linker region which blocks polymerase read through; or
   c. a 5′ terminal group other than 5′ phosphate.
IV. A fourth region of nucleotides which is either
   a. covalently attached with the third region, or
   b. in the event that the third region is a 5′ terminal group other than 5′-phosphase is discontinuous with the fourth region,
V. A fifth region of nucleotides which are complementary to a 5′ region of the modified oligonucleotide, or may optionally be a region of degenerate nucleotides;
VI. A 5′ blocking group—i.e. a 5′ group other than 5′ phosphate.
   wherein the second region and the fourth region comprise complementary regions of nucleotides which are capable of forming a duplex between the second a fourth regions.

In some embodiments, the first region (I) comprises 2-20 RNA nucleotides, such as 3-10 RNA nucleotides.

In some embodiments, the second region (II) comprises a sequencing primer binding site and/or a flow cell binding site.

In some embodiments, the second region (II) further comprises a molecular bar-code region. In some embodiments, the second region (II) further comprises a sequencing reaction bar-code region.

In some embodiments, the second region (II) further comprises a sequencing primer binding region.

In some embodiments, the 5′ capture probe comprises a linker region III which prevents polymerase read through during first strand synthesis.

In some embodiments, the linker region III comprises a non-nucleotide linker, such as a C₆₋₃₂ polyethyleneglycol linker, such as a C18 polyethyleneglycol linker or an alkyl linker, or is a region of nucleotides comprising an inverted nucleoside linkage.

In some embodiments, region III comprises or is a discontinuity resulting in a two stranded 5′ capture probe, the first strand comprising regions I and II, and a second strand comprising regions IV and V.

In some embodiments, region V comprises a predetermined region of nucleotides which are complementary to the 5′ region of a modified oligonucleotide.

In some embodiments, region V comprises a degenerate sequence of nucleotides.

In some embodiments, the 5′ terminus of region V comprises a fluorescent label such as 5′-FAM.

Blocks DNA Polymerase

A modification or linker moiety which blocks DNA polymerase prevents the read through of the polymerase across the linker moiety or modification, resulting in the termination of chain elongation.

Specific Primers

A specific primer is a primer which comprises the complementary sequence to the primer binding site. It will be understood that the term “specific” with regards a primer and a primer binding site may need to take into account to the template molecule to be used, i.e. a primer binding site in a capture probe or an adapter may in some embodiments be engineered so as to present the primer binding site in a complementary nucleic acid molecule prepared from the nucleic acid molecule which comprises the capture probe or adapter.

First Primer

As used herein, the first primer refers to the primer which is specific for a region of the capture probe which when hybridized to the capture probe oligonucleotide/modified oligonucleotide ligation product is used to initiate the polymerase mediated chain elongation (first strand synthesis), such as regions D or E as described herein. The first primer therefore comprises a sequence which is complementary to a region on the capture probe oligonucleotide, and may further comprise further regions, such as a sequencing primer binding site. The first primer may further comprise a binding site for binding to a solid support used in massively parallel sequencing, such as a flow cell binding site. The first primer may further comprise a PCR primer binding site for use in an amplification step (PCT step) where including in the method of the invention. The first primer may for example be 15-30 nucleotides in length and may for example be a DNA oligonucleotide primer.

As described herein, in some embodiments, the capture probe is self-priming, and no exogenously added first primer is required to initiate first strand synthesis.

Polymerase Mediated 5′-3′ Chain Elongation

As used herein, the polymerase mediated 5-3′ chain elongation refers to the polymerase mediated elongation of a complementary strand of the capture probe oligonucleotide/modified oligonucleotide ligation product from the first primer when hybridized to the capture probe oligonucleotide/modified oligonucleotide ligation product, a process which may be mediated by nucleic acid polymerases such as DNA polymerases or reverse transcriptase enzymes. As illustrated herein, the examples provide assays which can be used to identify suitable polymerase enzymes and experimental conditions which are capable of reading through (i.e. reverse transcribing across) the modified oligonucleotide. The polymerase is therefore an enzyme which is capable of reverse transcribing across the modified oligonucleotide sequence to provide an elongation product which comprises the complementary sequence of the entire modified oligonucleotide. In some embodiments, the polymerase is an enzyme which is capable of reverse transcribing across a LNA modified oligonucleotide sequence, such as an LNA phosphorothioate oligonucleotide sequence. In some embodiments, the modified oligonucleotide comprises at least two contiguous LNA nucleosides which are linked by a phosphorothioate internucleoside linkage. In some embodiments, the modified oligonucleotide comprises at least two contiguous sugar modified nucleosides which are linked by a phosphorothioate internucleoside linkage. In some embodiments, the modified oligonucleotide comprises at least two contiguous sugar modified nucleosides which are linked by a phosphorothioate internucleoside linkage, wherein at least one of the sugar modified nucleosides is a LNA nucleoside. In some embodiments, the modified oligonucleotide comprises at least two contiguous 2′-O-methoxyethyl nucleosides which are linked by a phosphorothioate internucleoside linkage. In some embodiments, the modified oligonucleotide comprises at least two contiguous sugar modified nucleosides which are linked by a phosphorothioate internucleoside linkage, wherein at least one of the sugar modified nucleosides is a LNA nucleoside and the other is a 2′-O-methoxyethyl nucleoside. In some embodiments the modified oligonucleotide comprises DNA and LNA nucleosides.

As illustrated in the examples, modified oligonucleotides, such as phosphorothioate and 2′ sugar modified oligonucleotides such as 2′-O-MOE or LNA oligonucleotides pose a considerable hurdle for polymerase enzymes. By screening numerous different DNA polymerases (including reverse transcriptases), the inventors have identified that the Volcano2G polymerase as highly effective in utilizing modified oligonucleotides as a template for DNA elongation. The inventors have also identified that Taq polymerase is also effective when used in the presence of polyethyleneglycol and/or propylene glycol. The droplet PCR methods used in the examples may be used to identify further suitable polymerase enzymes and enzyme conditions which may also be used in the methods of the invention.

Volcano2G polymerase is available from myPOLS Biotec GmbH (DE).

In some embodiments, the polymerase used in the method of the invention is a DNA polymerase based on wild-type Thermus aquaticus (Taq) DNA polymerase, comprising the mutations S515R, 1638F, and M747K with regard to the amino acid sequence of wild-type Taq. The amino acid sequence of Taq polymerase is provided as SEQ ID NO 1. In some embodiments, the polymerase is selected from the group consisting of (SEQ ID NO: 1) or an effective polymerase which has at least 70% identity such as at least 80% identity, such as at least 90% identity, such as at least 95% identity, such as at least 98% identity thereto. Effective DNA polymerases may be determined using the methods provided in the examples (e.g. by droplet PCR).

>sp|P19821|DPO1_THEAQ DNA polymerase I,
thermostable OS = Thermus aquaticus OX = 271 GN =
polA PE = 1 SV = 1
(SEQ ID NO 1)
MRGMLPLFEPKGRVLLVDGHHLAYRTFHALKGLTTSRGEPVQAVYGFAKS
LLKALKEDGDAVIVVFDAKAPSFRHEAYGGYKAGRAPTPEDFPRQLALIK
ELVDLLGLARLEVPGYEADDVLASLAKKAEKEGYEVRILTADKDLYQLLS
DRIHVLHPEGYLITPAWLWEKYGLRPDQWADYRALTGDESDNLPGVKGIG
EKTARKLLEEWGSLEALLKNLDRLKPAIREKILAHMDDLKLSWDLAKVRT
DLPLEVDFAKRREPDRERLRAFLERLEFGSLLHEFGLLESPKALEEAPWP
PPEGAFVGFVLSRKEPMWADLLALAAARGGRVHRAPEPYKALRDLKEARG
LLAKDLSVLALREGLGLPPGDDPMLLAYLLDPSNTTPEGVARRYGGEWTE
EAGERAALSERLFANLWGRLEGEERLLWLYREVERPLSAVLAHMEATGVR
LDVAYLRALSLEVAEEIARLEAEVERLAGHPFNLNSRDQLERVLFDELGL
PAIGKTEKTGKRSTSAAVLEALREAHPIVEKILQYRELTKLKSTYIDPLP
DLIHPRTGRLHTRFNQTATATGRLSSSDPNLQNIPVRTPLGQRIRRAFIA
EEGWLLVALDYSQIELRVLAHLSGDENLIRVFQEGRDIHTETASWMFGVP
REAVDPLMRRAAKTINFGVLYGMSAHRLSQELAIPYEEAQAFIERYFQSF
PKVRAWIEKTLEEGRRRGYVSTLFGRRRYVPDLEARVKSVREAAERMAFN
MPVQGTAADLMKLAMVKLFPRLEEMGARMLLQVHDELVLEAPKERAEAVA
RLAKEVMEGVYPLAVPLEVEVGIGEDWLSAKE

In some embodiments, the polymerase is a DNA polymerase having at least 80%, at least 90%, at least 95%, or at least 99% identity to the Taq polymerase having the amino acid sequence of SEQ ID NO:1 or its Klenow fragment, wherein the DNA polymerase comprises at least one amino acid substitution at one or more positions corresponding to position(s) 487, 508, 536, 587 and/or 660 of the amino acid sequence of the Taq polymerase shown in SEQ ID NO:1 of the Klenow fragment. See WO2015/082449, hereby incorporated by reference, including specifically the polymerases disclosed as SEQ ID NO 3-24. In some embodiments the DNA polymerase has at least 80% complementarity to SEQ ID NO 1, such as at least 90% complementarity to SEQ ID NO 1 and comprises wherein said one or more amino acid substitution is selected from the group consisting of R487HN, K508W/Y, R536K/L, R587K/I, and R660TN for SEQ ID NO:1.

Adapter Probe

As used herein, the term “adapter probe” refers to the oligonucleotide probe which is ligated to the 3′ end of the elongation product from the polymerase mediated 5′-3′ chain elongation from the first primer. The adapter probe provide a primer binding site which may be used directly for primer based sequencing, and/or may be used in an amplification step (PCT step) where including in the method of the invention. The adapter probe may further comprise further regions, such as a sequencing primer binding site. The adapter probe may further comprise a binding site for binding to a solid support used in massively parallel sequencing, such as a flow cell binding site. The adapter probe may further comprise a PCR primer binding site for use in an amplification step (PCR step) where including in the method of the invention.

PCR Amplification

In some embodiments, after the ligation of the adapter probe to the 3′ end of the elongation product a PCR amplification step is performed. The PCR amplification uses a pair of PCR primers, wherein one of the primers is specific for a region on the capture probe (may be the first primer binding sequence or a region of the capture probe upstream of the first primer binding sequence), and the other PCR primer is specific for a region of the adapter probe. In some embodiments, such as in parallel sequencing embodiments, the PCR amplification is performed using primers which are attached to a solid surface, such as on-bead amplification or solid phase bridge amplification. In some embodiments the solid phase is a flowcell (e.g. as used in solid phase bridge amplification, e.g. as used in the Illumina sequencing platform). Solid phase PCR used in solid phase bridge amplification is also referred to as cluster generation: A library of products obtained from the ligation of the adapter probes are captured on a lawn of surface-bound oligonucleotides complementary to a region of the adapter probe and/or the capture probe (flow cell binding sites). Each fragment is then amplified into distinct, clonal clusters through bridge amplification. When cluster generation is complete, the templates are ready for sequencing by synthesis.

The number of PCR cycles may in some embodiments be limited so that each cluster has about 1000 copies. In some embodiments, the PCR step utilizes reduced cycle PCR, i.e. the number of PCR cycles is limited to between 2 and about 25 cycles, such as about 10 to about 20 PCR cycles.

Bar Coding

A bar code is a sequence within a capture probe or primer which is used to identify the original of a sequence obtained in the methods of the invention, e.g. with regards identification of multiple sequences which originate from the same capture probe ligation event (molecular bar code) or from a common capture probe ligation reaction (reaction bar code).

Molecule Bar-Code (e.g. May be Used in Region C of the Capture Probe)

In some embodiments, the capture probe oligonucleotides and/or the adapter probe comprises a sequence of random nucleoside sequence (a degenerate sequence). The use of a degenerate sequence within the capture or adapter probe can be used to allow for the identification of sequencing results which result from duplication of the same ligated elongation product molecule after a PCR amplification step.

Reaction Bar-Code (e.g. as Used in Region b of the Capture Probe)

The capacity of massively parallel sequencing enables the pooling of sequencing templates into a single sequencing experiment, thereby enhancing the cost effectiveness of each sequencing run. It is therefore desirable to be able to separate sequencing data to identify the sequences which originate from separate sequencing template reaction. This may be achieved by using capture probes or PCR primers which incorporate a common sequence identify which is unique to each template. The length of the reaction bar code can be modified to reflect the complexity of different sequencing templates pooled into each parallel sequencing run, and may for example be 2-20 nucleotides (e.g. DNA nucleotides in length), such as 4-5 nucleotides in length.

Degenerate Nucleotides

A degenerate nucleotide refers to a position on a nucleic acid sequence that can have multiple alternative bases (as used in the IUPAC notation of nucleic acids) at a defined position. It should be recognized that for an individual molecule there will be a specific nucleotide at the defined position, but within the population of molecules in the oligonucleotide sample, the nucleotide at the defined position will be degenerate. In effect, the incorporation of the degenerate sequence results in the randomization of nucleotide sequence at the defined positions between each members of a population of oligonucleotides. It is known that some sequences may be preferentially amplified during PCR, and as such by counting the occurrence of a genetic “barcode sequence”, originating from the degenerate sequence, you can determine the pre-amplification relative quantities (see e.g. Kielpinski & Vinter, NAR (2014) 42 (8): e70. In some embodiments the capture probe comprises a region of universal based (e.g. inosine nucleotides) which may be used in place of degenerate nucleotides.

Sequencing

Sequencing refers to the determination of the order (sequence) of nucleobases within a nucleic acid molecule. In the context of the present invention sequencing refers to the determination of the sequence of nucleobases within a modified oligonucleotide. Traditional sequencing methods are based on the chain-termination method (known as Sanger sequencing) which uses selecting incorporation of chain-terminating dideoxynucleotides by DNA polymerase during in vitro DNA replication, followed by electrophoresis separation of the chain terminated products. By use of four separate reactions, each with a different chain terminating base (A, T, C or G), the sequence is determined by comparing the relative motility of the 4 chain termination reaction products in gel-electrophoresis.

Sanger sequencing was initially developed based on the incorporation of radiolabeled nucleotides followed by SDS-PAGE electrophoresis, and was commercially developed as the basis for automated DNA sequencing using primers labelled with a fluorescent dye, which, for example could be detected by capillary electrophoresis. The use of dye-terminator sequencing allowed the sequencing from a single reaction mixture (rather than the four reactions of the original Sanger method), enabling automation. In some embodiments, the sequencing step of the method of the invention is performed using automated sequencing. In some embodiments, the sequencing step of the method of the invention is performed using dye-terminator sequencing such as automated dye terminator sequencing.

Whilst Sanger based sequencing is still employed today, for large scale sequencing applications, it has been superseded by “Next Generation” sequencing technologies, see Goodwin et al., Nature Reviews: Genetics Vol 17 (2016), 333-351, hereby incorporated by reference.

Primer Based Sequencing

Primer based sequencing refers to the use of 5′-3′ polymerase based chain elongation from a primer hybridized to the nucleic acid template. Primer based sequencing may be based upon the chain termination method (e.g. Sanger sequencing) or advantageously using sequencing by synthesis.

Capture Probe/Adapter Based Sequencing

The present invention provides a method for sequencing a modified oligonucleotides or population of modified oligonucleotides. In some embodiments, the method comprises the step of ligating a capture probe to the modified oligonucleotide, followed by the hybridization of a first primer which is complementary to the capture probe, which is subsequently used for polymerase based chain elongation to produce an elongation product. An adapter is then ligated to the 3′ end of the elongation product, resulting in a nucleic acid molecule which comprises the complementary sequence of the modified oligonucleotide flanked 5′ and 3′ by known probe sequences, which may be used as primer binding sites, e.g. which may be used directly in primer based sequencing (single molecule template sequencing) or may be amplified prior to sequencing, e.g. via PCR or reduced cycle amplification (clonal amplification sequencing).

In some embodiments, the sequencing step is performed using “sequencing by a synthesis” method.

Sequencing by Synthesis

Whereas traditional Sanger based sequencing is based upon chain-termination, sequencing by synthesis is based upon the addition of dye labelled nucleotides during chain elongation without initiating chain termination. By real time monitoring of unique dye signals (one for each of the four bases, A, T, C and G), the sequence is captured during chain elongation. A notable advantage of sequencing by synthesis methods is that it allows for massively parallel sequencing of a complex mixture of nucleic acid sequences. In some embodiments the sequencing method use in the method of the invention is a cyclic reversible termination method or a single-nucleotide addition method.

Sequencing by synthesis methods are typically based upon cyclic reversible termination (CRT) or single-nucleotide addition (SNA) approaches (Metzker, M. L. Sequencing technologies—the next generation. Nat. Rev. Genet. 11, 31-46 (2010):

Cyclic reversible termination (CRT) methods, as used by the Illumina NGS platform and the Qiagen Intelligent BioSystems/GeneReader platforms, use reversible terminator molecules in which the ribose 3″ —OH group is blocked, thus preventing elongation. To begin the process, a DNA template is primed by a sequence that is complementary to an adapter region, which will initiate polymerase binding to this double-stranded DNA (dsDNA) region. During each cycle, a mixture of all four individually labelled and 3″-blocked deoxynucleotides (dNTPs) are added. After the incorporation of a single dNTP to each elongating complementary strand, unbound dNTPs are removed and the surface is imaged to identify which dNTP was incorporated at each cluster. The fluorophore and blocking group can then be removed and a new cycle can begin.

Clonal Bridge Amplification is employed by the Illumina system, as used in the examples herein. In some embodiments, the sequencing method used in the methods of the invention is clonal bridge amplification.

Single-nucleotide addition methods, as used by the 454 pyrosequencing system (Roche) and Ion Torrent NGS system, rely on a single signal to mark the incorporation of a dNTP into an elongating strand. As a consequence, each of the four nucleotides must be added iteratively to a sequencing reaction to ensure only one dNTP is responsible for the signal. Furthermore, this does not require the dNTPs to be blocked, as the absence of the next nucleotide in the sequencing reaction prevents elongation. The exception to this is homopolymer regions where identical dNTPs are added, with sequence identification relying on a proportional increase in the signal as multiple dNTPs are incorporated. Notably the Ion Torrent system does not use fluorescent nucleotides, but instead detects the H+ ions that are released as each dNTP is incorporated. The resulting change in pH is detected by an integrated complementary metal-oxide-semiconductor (CMOS) and an ion-sensitive field-effect transistor (ISFET).

Parallel Sequencing

Whereas in traditional Sanger sequencing each sequencing run is used to determine the sequence of a single nucleic acid template, the employment of next generation sequencing methods allows for parallel sequencing of heterogenous mixtures of nucleic acid sequences. As described herein, parallel sequencing can employ a clonal amplification step, and by incorporation of sequence based identifiers within the amplification primers, the repeated clonal sequences originating from each original template molecule can be identified.

Whilst massively parallel sequencing has primarily been developed to enable the rapid and efficient sequencing of long polynucleotide sequences, including entire chromosomes and genomes, enabling individual genotyping solutions, the present inventors have identified that these solutions also provide the unique opportunity to identify the presence and comparative abundance of individual molecular species within a population of modified oligonucleotides. Such methods are useful in numerous applications, such as oligonucleotide therapeutic discovery, manufacture & quality assurance, therapeutic development, and patient monitoring.

### DETAILED DESCRIPTION OF THE INVENTION

The invention provides for methods for sequencing a modified oligonucleotide such as 2′ sugar modified oligonucleotide, such as a LNA oligonucleotide or a 2-O-methoxyethyl oligonucleotide.

If the 5′ and 3′ regions of the modified oligonucleotide are known, the method of the invention may be used to sequence between the two known regions to identify the sequence between the two known regions, e.g. an aptameric region. This may be achieved by using primers which are specific for the known regions of the modified oligonucleotide (e.g. PCR or sequencing primers), or advantageously may be achieved using capture probes as described herein.

However, the methods of the invention may also be used to sequence modified oligonucleotides where the nucleotide sequence is not known, or only a 5′ region or a 3′ sequence is known. This is may be achieved by using the 5′ and/or 3′ capture probes described herein.

When a 3′ region of the modified oligonucleotide is known, and the method is to determine the sequence of the modified oligonucleiotide upstream (5′) to the known 3′ region, the method of the invention may be performed by ligating a 5′ capture probe to the modified oligonucleotide and performing first strand synthesis using a primer which is specific to the known 3′ region.

When a 5′ region of the modified oligonucleotide is known, and the method is to determine the sequence of the modified oligonucleotide downstream (3′) to the known 5′ region, the method of the invention may be performed by ligating a 3′ capture probe to the modified oligonucleotide and performing first strand synthesis using a primer which is specific to the 3′ capture probe.

The method may comprise the step of ligating a 3′ capture probe to the modified oligonucleotide, followed by first strand synthesis. After first strand synthesis an adapter primer may be ligated to the 3′ end of the elongation product, and primer based sequencing may be performed using primers which are specific for the 3′ capture probe and the adapter probe, or the first strand synthesis product may be PCR amplified using primers which are specific for the 3′ capture probe and adaptor probe.

The method may comprise the step of ligating a 3′ capture probe and a 5′ capture probe to the modified oligonucleotide, followed by first strand synthesis. Primer based sequencing may be performed using primers which are specific for the 3′ and 5′ capture probes, or the first strand synthesis product may be PCR amplified using primers which are specific for the 5′ & 3′ capture probes.

The method may comprise the step of ligating a 3′ capture probe, performing first strand synthesis and then ligating an adaptor probe to the 3′ terminus of the first strand synthesis product.

The method may comprise the step of ligating a 3′ capture probe to the modified oligonucleotide, wherein the 3′ capture probe is used to initiate first strand synthesis of the modified oligonucleotide template also referred to as reverse transcription, or 5′-3′ chain elongation), resulting in a first strand synthesis product which comprises the complementary sequence to the modified oligonucleotide and suitably a region complementary to a region of the 3′ capture probe (this region may be used as a PCR primer/clonal amplification primer binding site or a flow cell primer binding site or sequencing primer binding site).

After first strand synthesis the first strand synthesis product may be PCR amplified, which may be a clonal amplification step. The PCR may use a first PCR primer which is specific for the capture probe derived region, and a second PCR primer which may, in some embodiments be specific for a region of the modified oligonucleotide (suitably a 5′ region, thereby allowing the sequencing of the remaining 3′ region of the modified oligonucleotide). Alternatively the whole modified oligonucleotide may be PCR amplified by further extending the first strand synthesis product at the 3′ end, for example by the ligation of an adapter probe which comprises a PCR primer binding site, or via poly nucleation (a region of polyN is added to the 3′ terminus), such as polyadenylation (or PolyC and polyU), a process which can be catalyzed using terminal transferase enzymes. A second strand synthesis may be performed using a second strand synthesis primer comprising the complementary polyN sequence. The second primer may comprise a second PCR primer binding site. The PCR step may then be performed using the first PCR primer which is specific for the 3′capture probe, and a second PCR primer which is either specific for the poly(N) sequence or the second strand synthesis primer PCR binding site. It will be understood that the PCR primer sites may be used for clonal amplification as part of the sequencing step of the method of the invention, or directly for primer based sequencing. An advantage of using the adaptor probe ligation embodiment or polynucleation embodiment is that it facilitates the sequencing of modified oligonucleotides where the 5′ region of the oligonucleotide is not known.

The length of the modified oligonucleotide may, for example, be up to 60 contiguous nucleotides, such as up to 50 contiguous nucleotides, such as up to 40 contiguous nucleotides. In some embodiments the modified oligonucleotide is or comprises a phosphorothioate oligonucleotide of 7-30 nucleotides in length. In some embodiments the modified oligonucleotide is or comprises a sugar modified phosphorothioate oligonucleotide of 7-30 nucleotides in length. In some embodiments the modified oligonucleotide is a 2′ sugar modified phosphorothioate oligonucleotide of 7-30 nucleotides in length. In some embodiments the modified oligonucleotide is a LNA oligonucleotide of 7-30 nucleotides in length. In some embodiments the modified oligonucleotide is a LNA phosphorothioate oligonucleotide of 7-30 nucleotides in length. In some embodiments the modified oligonucleotide comprises one or more LNA nucleoside, or one or more 2′-O-methoxyethyl nucleoside. In some embodiments, the 3′ most nucleoside of the modified oligonucleotide is a LNA nucleoside. In some embodiments the 3′ most nucleoside of the modified oligonucleotide is a 2′ substituted nucleoside such as a 2′-O-methyoxyethyl or 2′-O-methyl nucleoside.

In some embodiments the sequencing step is performed using sequencing by synthesis method.

In some embodiments, the chain elongation step, also referred to as polymerase mediated 5′-3′ first strand synthesis, is performed in the presence of a polymerase and polyethylene glycol (PEG) or propylene glycol. In such embodiments, the polymerase may, optionally be a Taq polymerase, such as the Taq polymerase shown as SEQ ID NO 1 or an effective polymerase which has at least 70% identity such as at least 80% identity, such as at least 90% identity, such as at least 95% identity, such as at least 98% identity thereto.

In some embodiments, the chain elongation step also referred to as polymerase mediated 5′-3′ first strand synthesis—is performed in the presence of a polymerase and polyethylene glycol (PEG) of mean molecule weight of 100-20,000, such as from about 2000 to about 10000, such as about 4000.

In some embodiments, the concentration of PEG in the chain elongation reaction (first strand synthesis step) is between about 2% & about 15% (w/v—i.e. weight of PEG/reaction volume), such as from about 3% to about 15%. Above 15% can still result in efficient elongation however in the droplet PCR system it results in destabilization of the droplets. In some embodiments the concentration of PEG is between about 2% and about 20%, or between about 3% and 30% (w/v).

In some embodiments the concentration of propylene glycol in the chain elongation reaction mixture (first strand synthesis step) is at least about 0.8M and may for example be between about 0.8M and 2M, such as between about 1M and about 1.6M.

As illustrated in the examples, the addition of PEG may provide more effective chain elongation/first strand synthesis than the addition of propylene glycol.

The use of PEG and/or propylene glycol has been found to be advantageous for use with a range of polymerases, for example Taq polymerases and polymerases derived from Taq polymerase as disclosed herein, for example Volcano2G polymerase. It is considered that the assays disclosed herein are to be used to identify further polymerase enzymes, and as required reaction conditions which provide effective first strand synthesis across the length of the modified oligonucleotide.

In some embodiments, the polymerase used for 5′-3′ chain elongation (first strand synthesis step) is a Taq polymerase, such as the Taq polymerases as describe herein or Volcano2G polymerase.

In some embodiments the polymerase is PrimeScript reverse transcriptase (available from Clontech).

The selection of the DNA polymerase/reverse transcriptase may be performed by evaluating the relative efficiency of the polymerase to read through the modified oligonucleotide, such as sugar-modified oligonucleotides. For sugar modified oligonucleotides, this may depend on the length of contiguous sugar-modified nucleosides in the oligonucleotide, and it is recognized that for heavily modified oligonucleotides an enzyme other than Taq polymerase may be desirable. The selection of the DNA polymerase/reverse transcriptase will also depend on the purity of the sample, it is well known that some polymerase enzymes are sensitive to contaminants, such as blood (See Al-Soud et al, Appl Environ Microbiol. 1998 October; 64(10): 3748-3753 for example).

Advantageously, the DNA polymerase is a Volcano2G DNA polymerase.

In some embodiments the first strand synthesis (elongation step) is performed using a reverse transcriptase. In some embodiments, the reverse transcriptase may be selected from the group consisting of M-MuLV Reverse Transcriptase, a modified M-MuLV Reverse Transcriptase, SuperScript™ III RT, AMV Reverse Transcriptase, Maxima H Minus Reverse Transcriptase. In some embodiments the DNA polymerase is a thermostable polymerase such as a DNA polymerase selected from the group consisting of Taq polymerase, Hottub polymerase, Pwo polymerase, rTth polymerase, Tfl polymerase, Ultima polymerase, Volcano2G polymerase, and Vent polymerase. It will be understood that for certain enzymes, in order to efficiently perform first strand synthesis of the modified oligonucleotide it may be necessary to optimize the reaction conditions, e.g. via the addition of PEG and/or propylene glycol.

Advantageously, the modified oligonucleotide is a phosphorothioate oligonucleotide. In some embodiments at least 75% of the internucleoside linkages within the modified oligonucleotide are phosphorothioate internucleoside linkages, such as at least 90% of the internucleoside linkages within the modified oligonucleotide are phosphorothioate internucleoside linkages, such as all the internucleoside linkages within the modified oligonucleotide are phosphorothioate internucleoside linkages.

In some embodiments, the modified oligonucleotide is a 2′ sugar modified oligonucleotide. In some embodiments, the modified oligonucleotide comprises at least 2′ sugar modified nucleosides. In some embodiments the modified oligonucleotide comprises at least 1 or at least 2 3′ terminal sugar modified nucleoside, such as at least 1 or at least 3′ terminal LNA nucleoside or at least 1 or at least 2 terminal 2′-O-MOE nucleosides. In some embodiments, the modified nucleoside comprises at least 3 2′ sugar modified nucleosides, such as 4, 5, 6, 7, 8, 9, 10 or more 2′ sugar modified nucleosides. In some embodiments the 2′ sugar modified nucleosides are independently selected from LNA nucleosides and 2′ substituted sugar modified nucleosides, such as 2′-O-MOE nucleosides. Advantageously, the modified oligonucleotide is a 2′ sugar modified phosphorothioate oligonucleotide, such as a LNA modified phosphorothioate oligonucleotide wherein at least 75% of the internucleoside linkages within the oligonucleotide are phosphorothioate internucleoside linkages and at least one of the nucleosides within the modified oligonucleotides is an LNA nucleoside, such as 2, 3, 4, 5, 6, 7, 8, 9, 10, 11 or 12 of the nucleosides within the modified oligonucleotide are LNA nucleosides. Advantageously the 3′ most nucleoside of the modified LNA oligonucleotide is a sugar modified nucleoside such as an LNA nucleoside or may be a 2′ substituted nucleoside such as a 2′-O-MOE nucleoside. In some embodiments the modified oligonucleotide comprises at least two contiguous LNA nucleosides.

In some embodiments, the modified oligonucleotide comprises at least one modified nucleoside selected from the group consisting of 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleoside.

In some embodiments, the modified oligonucleotide comprises at least one 2′-O-methoxyethyl RNA (MOE) nucleoside. In some embodiments, the modified oligonucleotide comprises at least one 3′ terminal 2′-O-methoxyethyl RNA (MOE) nucleoside and at least one further 2′-O-methoxyethyl RNA (MOE) nucleoside.

In some embodiments the modified oligonucleotide is a 2′-O-MOE modified phosphorothioate oligonucleotide, such as a 2′-O-MOE modified phosphorothioate oligonucleotide wherein at least 75% of the internucleoside linkages within the oligonucleotide are phosphorothioate internucleoside linkages and at least one of the nucleosides within the modified oligonucleotides is an 2′-O-MOE nucleoside, such as 2, 3, 4, 5, 6, 7, 8, 9, 10, 11 or 12 of the nucleosides within the modified oligonucleotide are 2′-O-MOE nucleosides. Advantageously the 3′ most nucleoside of the modified 2′-O-MOE oligonucleotide is a sugar modified nucleoside such as an 2′-O-M0E. In some embodiments the modified oligonucleotide comprises at least two contiguous 2′-O-MOE nucleosides, such as at least 3, 4 or 5 contiguous 2-O-MOE nucleosides.

In some embodiments, the modified oligonucleotide is a population of modified oligonucleotides, which mat for example be from the same oligonucleotide synthesis run or from a pool of oligonucleotide synthesis runs. During oligonucleotide synthesis or manufacture, each oligonucleotide synthesis run will comprise a population of oligonucleotide species, for example the desired oligonucleotide product as well as truncated versions, e.g. so called n−1 products. Furthermore, as illustrated herein, within the population of oligonucleotide species, oligonucleotides with a different sequence may arise due to impurities in the monomers used in the synthesis run or contamination of the synthesis column from previous coupling cycles. It is therefore important to characterize the presence of these impurities at a sequence level. In some embodiments the population of modified oligonucleotides is obtained from a series of synthesis runs where the products of each synthesis run are pooled for form a single batch of modified oligonucleotide which can be tested by the methods of the present invention.

In some embodiments, the 2′ sugar modified oligonucleotide is a 2′ sugar modified phosphorothioate oligonucleotide.

In some embodiments, the modified oligonucleotide comprises at least two contiguous 2′ sugar modified nucleosides.

In some embodiments, the modified oligonucleotide comprises at least one 2′-O-methoxyethyl RNA (MOE) nucleoside.

In some embodiments, the modified oligonucleotide comprises at least two contiguous 2′-O-methoxyethyl RNA (MOE) nucleosides.

In some embodiments, the modified oligonucleotide comprises at least one 2′-O-methoxyethyl RNA (MOE) nucleoside located at the 3′ of the modified oligonucleotide, such as at least two or at least three contiguous 2′-O-methoxyethyl RNA (MOE) nucleosides located at the 3′ end of the modified oligonucleotide.

In some embodiments, the modified oligonucleotide comprises at least 1 LNA nucleoside. In some embodiments, the modified oligonucleotide comprises at least two contiguous LNA nucleotides or at least three contiguous LNA nucleotides.

In some embodiments, the LNA nucleotide(s) are located at the 3′ end of the LNA oligonucleotide.

In some embodiments, the modified oligonucleotide is a LNA phosphorothioate oligonucleotide.

In some embodiments, the modified oligonucleotide comprises both LNA nucleosides and DNA nucleosides, such as a LNA gapmer, or LNA mixmer.

In some embodiments, the modified oligonucleotide comprises at least one T nucleoside or at least one C nucleoside, such as at least one DNA-C or at least one DNA-T, or at least one 2′-methoxyethyl (MOE) C nucleoside or at least one 2′-methoxyethyl (MOE) T nucleoside. In some embodiments, the LNA oligonucleotide comprises at least one LNA-T nucleoside or at least one LNA-C nucleoside.

In some embodiments, the modified oligonucleotide comprises one or more LNA nucleoside(s) and one or more 2′substituted nucleoside, such as one or more 2′-O-methoxyethyl nucleosides.

In some embodiments, the modified oligonucleotide is selected from the group consisting of; a 2′-O-methoxyethyl gapmer, a mixed wing gapmer, an alternating flank gapmer or a LNA gapmer.

In some embodiments, the modified oligonucleotide is a mixmer or a totalmer.

In some embodiments, the modified oligonucleotide comprise a conjugate group, such as a GalNAc conjugate.

In some embodiments, the sequencing step uses massively parallel sequencing.

In some embodiments, the template for primer based sequencing (PCR step of the method of the invention), such as massively parallel sequencing, is performed using clonal bridge amplification (e.g. Illumina sequencing—reversible dye terminator), or clonal emPCR (emulsion PCR, e.g. Roche 454, GS FLX Titanium, Life Technologies SOLiD4, Life Technologies Ion Proton). In some embodiments, the template for primer based sequencing is performed using solid-phase template walking (e.g. SOLiD Wildfire, Thermo Fisher).

Massively Parallel Sequencing Platforms (Next Generation Sequencing) are Commercially available—for example as illustrated in the table below (as listed in Wikipedia):

Exemplary NGS Platforms

| | | | Max Read | Run | |
|---|---|---|---|---|---|
| | Template | | length | Times | Max Gb |
| Platform | Preparation | Chemistry | (bases) | (days) | per Run |
| Roche 454 | Clonal-emPCR | Pyrosequencing | 400‡ | 0.42 | 0.40-0.60 |
| GS FLX | Clonal-emPCR | Pyrosequencing | 400‡ | 0.42 | 0.035 |
| Titanium | | | | | |
| IIlumina MiSeq | Clonal Bridge | Reversible Dye | 2 × 300 | 0.17-2.7 | 15 |
| | Amplification | Terminator | | | |
| IIlumina HiSeq | Clonal Bridge | Reversible Dye | 2 × 150 | 0.3-11 | 1000 |
| | Amplification | Terminator | | | |
| IIlumina | Clonal Bridge | Reversible Dye | 2 × 150 | 2-14 | 95 |
| Genome | Amplification | Terminator | | | |
| Analyzer IIX | | | | | |
| Life | Clonal-emPCR | Oligonucleotide 8- | 35-50 | 4-7 | 35-50 |
| Technologies | | mer Chained | | | |
| SOLiD4 | | Ligation^{[} | | | |
| Life | Clonal-emPCR | Native dNTPs, proton | 200 | 0.5 | 100 |
| Technologies | | detection | | | |
| Ion Proton | | | | | |
| Complete | Gridded DNA- | Oligonucleotide 9- | 7 × 10 | 11 | 3000 |
| Genomics | nanoballs | mer Unchained | | | |
| | | Ligation^{[17][18][19]} | | | |
| Helicos | Single | Reversible Dye | 35‡ | 8 | 25 |
| Biosciences | Molecule | Terminator | | | |
| Heliscope | | | | | |
| Pacific | Single | Phospholinked | 10,000 | 0.08 | 0.5 |
| Biosciences | Molecule | Fluorescent | (N50); | | |
| SMRT | | Nucleotides | 30,000+ | | |
| | | | (max) | | |

In some embodiments, after ligation of the 3′ capture probe to the modified oligonucleotide, the ligation product is purified, e.g. via gel purification, or via enzymatic degradation of the un-ligated capture probe, prior to first strand synthesis (chain elongation).

In some embodiments, after ligation of the adapter probe to the first strand synthesis product, the ligation product is purified, e.g. via gel purification, or via enzymatic degradation of the un-ligated capture probe, prior to PCR or sequencing steps.

In some embodiments, the modified oligonucleotide/3′capture probe ligation product is purified, e.g. via gel purification, or via enzymatic degradation of the un-ligated capture probe.

In some embodiments, the first strand synthesis strand/adapter probe ligation product is purified, e.g. via gel purification, or via enzymatic degradation of the un-ligated capture probe.

In some embodiments, the capture probe or adapter probe or both, each comprise sequencing primer binding sites.

In some embodiments, the first primer or the adapter probe or both, each comprise sequencing primer binding sites.

In some embodiments, the method comprises a PCT amplification step, one or both of the PCR primers used in the PCR step comprise sequencing primer binding sites.

In some embodiments, the capture probe and adapter probe, or the first primer and the adapter probe, further comprise flow cell binding sites.

In some embodiments, the PCR primers used in the PCR step further comprise flow cell binding sites.

Exemplary Modified Oligonucleotide Embodiments

The modified oligonucleotides may be phosphorothioate oligonucleotides. The modified oligonucleotides may be phosphorothioate sugar modified oligonucleotides, such as phosphorothioate 2′sugar modified oligonucleotides, such as an LNA phosphorothioate oligonucleotide or a 2′-O-methoxyethyl (MOE) phosphorothioate oligonucleotide.

In some embodiments, the modified oligonucleotide is a therapeutic oligonucleotide.

In some embodiments the modified oligonucleotide, comprises a conjugate moiety, such as a N-Acetylgalactosamine (GalNAc) moiety, such as a trivalent GalNAc moiety.

In some embodiments the modified oligonucleotide is an LNA oligonucleotide which comprises a conjugate moiety, such as a N-Acetylgalactosamine (GalNAc) moiety, such as a trivalent GalNAc moiety.

In some embodiments, the modified oligonucleotide(s) is a gomer oligonucleotide, such as a MOE gapmer, a LNA gapmer, a mixed wing gapmer or an alternating flank gapmer. In some embodiments the modified oligonucleotide is a mixmer oligonucleotide, such as an LNA mixmer oligonucleotide. In some embodiment the modified oligonucleotide is a totalmer, such as a MOE totalmer, or an LNA totalmer oligonucleotide.

In some embodiments the modified oligonucleotide is a sugar modified oligonucleotide, such as an oligonucleotide comprising LNA or 2′-O-methoxyethyl modified nucleosides, or both LNA and 2′-O-methoxyethyl modified nucleotides.

In some embodiments, the modified oligonucleotide is a LNA phosphorothioate oligonucleotide.

In some embodiments, the modified oligonucleotide comprises both LNA nucleosides and DNA nucleosides, such as a LNA gapmer, or LNA mixmer. In some embodiments, the modified oligonucleotide comprises at least one beta-D-oxy LNA nucleoside or at least one (S)cET LNA nucleoside (6′methyl beta-D-oxyLNA). In some embodiments, the LNA nucleosides present in the LNA oligonucleotide are either beta-D-oxy LNA nucleoside or at least one (S)cET LNA nucleoside (6′methyl beta-D-oxy LNA).

In some embodiments, the modified oligonucleotide comprises at least one sugar modified T nucleoside and/or at least one sugar modified C residue (Including 5 methyl C).

In some embodiments, the modified oligonucleotide comprises at least one LNA-T nucleoside and/or at least one LNA-C (Including 5-methyl C) nucleoside.

In some embodiments, the modified oligonucleotide comprises at least one 2′-O-methoxyethyl T nucleoside and/or at least one 2′-O-methoxyethyl C residue (Including 5 methyl C). The synthesis of cytosine and thymine phosphoramidite monomers used in oligonucleotide synthesis is often via common intermediates—and as illustrated in the examples, this can result in the contamination between C or T phosphoramidites, a problem which the methods of the invention are able to detect.

In some embodiments, the nucleoside modified oligonucleotide comprises at least one (such as 1, 2, 3, 4 or 5) 3′ terminal modified nucleosides, such as at least one (such as 1, 2, 3, 4 or 5) LNA or at least one (such as 1, 2, 3, 4 or 5) 2′ substituted nucleosides, such as 2′O -MOE. In some embodiments, the nucleoside modified oligonucleotide comprises at least one non terminal modified nucleosides, such as LNA or a 2′ substituted nucleoside, such as 2′-O-MOE.

In some embodiments, the modified oligonucleotide comprises one or more LNA nucleoside(s) and one or more 2′substituted nucleoside, such as one or more 2′-O-methoxyethyl nucleosides.

In some embodiments, the modified oligonucleotide comprise a conjugate group, also referred to as a conjugate moiety, such as a GalNAc conjugate. In some embodiments the conjugate moiety is positioned at a terminal position in the modified oligonucleotide, such as at the 3′ terminus or the 5′ terminus, and there may be a nucleosidic or non nucleosidic linker moiety covalently connecting the conjugate group to the oligonucleotide.

The Conjugate Moiety

In some embodiment the conjugate moiety is selected from the group consisting of a protein, such as an enzyme, an antibody or an antibody fragment or a peptide; a lipophilic moiety such as a lipid, a phospholipid, a sterol; a polymer, such as polyethyleneglycol or polypropylene glycol; a receptor ligand; a small molecule; a reporter molecule; and a non-nucleosidic carbohydrate.

In some embodiments, the conjugate moiety comprises or is a carbohydrate, non nucleosidic sugars, carbohydrate complexes. In some embodiments, the carbohydrate is selected from the group consisting of galactose, lactose, n-acetylgalactosamine, mannose, and mannose-6-phosphate.

In some embodiments, the conjugate moiety comprises or is selected from the group of protein, glycoproteins, polypeptides, peptides, antibodies, enzymes, and antibody fragments, In some embodiments, the conjugate moiety is a lipophilic moiety such as a moiety selected from the group consisting of lipids, phospholipids, fatty acids, and sterols.

In some embodiments, the conjugate moiety is selected from the group consisting of small molecules drugs, toxins, reporter molecules, and receptor ligands.

In some embodiments, the conjugate moiety is a polymer, such as polyethyleneglycol (PEG), polypropylene glycol.

In some embodiments the conjugate moiety is or comprises a asialoglycoprotein receptor targeting moiety, which may include, for example galactose, galactosamine, N-formyl-galactosamine, Nacetylgalactosamine, N-propionyl-galactosamine, N-n-butanoyl-galactosamine, and N-isobutanoylgalactos-amine. In some embodiments the conjugate moiety comprises a galactose cluster, such as N-acetylgalactosamine trimer. In some embodiments, the conjugate moiety comprises a GalNAc (N-acetylgalactosamine), such as a mono-valent, di-valent, tri-valent of tetra-valent GalNAc. Trivalent GalNAc conjugates may be used to target the compound to the liver (see e.g. U.S. Pat. No. 5,994,517 and Hangeland et al., Bioconjug Chem. 1995 November-December; 6(6):695-701, WO2009/126933, WO2012/089352, WO2012/083046, WO2014/118267, WO2014/179620, & WO2014/179445).

Conjugate Linkers

A linkage or linker is a connection between two atoms that links one chemical group or segment of interest to another chemical group or segment of interest via one or more covalent bonds. Conjugate moieties can be attached to the oligonucleotide directly or through a linking moiety (e.g. linker or tether). Linkers serve to covalently connect a third region, e.g. a conjugate moiety to an oligonucleotide (e.g. the termini of region A or C).

In some embodiments of the invention the conjugate or oligonucleotide conjugate of the invention may optionally, comprise a linker region which is positioned between the oligonucleotide and the conjugate moiety. In some embodiments, the linker between the conjugate and oligonucleotide is biocleavable.

Biocleavable linkers comprising or consisting of a physiologically labile bond that is cleavable under conditions normally encountered or analogous to those encountered within a mammalian body. Conditions under which physiologically labile linkers undergo chemical transformation (e.g., cleavage) include chemical conditions such as pH, temperature, oxidative or reductive conditions or agents, and salt concentration found in or analogous to those encountered in mammalian cells. Mammalian intracellular conditions also include the presence of enzymatic activity normally present in a mammalian cell such as from proteolytic enzymes or hydrolytic enzymes or nucleases. In one embodiment the biocleavable linker is susceptible to S1 nuclease cleavage. In a preferred embodiment the nuclease susceptible linker comprises between 1 and 10 nucleosides, such as 1, 2, 3, 4, 5, 6, 7, 8, 9 or 10 nucleosides, more preferably between 2 and 6 nucleosides and most preferably between 2 and 4 linked nucleosides comprising at least two consecutive phosphodiester linkages, such as at least 3 or 4 or 5 consecutive phosphodiester linkages. Preferably the nucleosides are DNA or RNA. Phosphodiester containing biocleavable linkers are described in more detail in WO 2014/076195 (hereby incorporated by reference).

Conjugates may also be linked to the oligonucleotide via non biocleavable linkers, or in some embodiments the conjugate may comprise a non-cleavable linker which is covalently attached to the biocleavable linker. Linkers that are not necessarily biocleavable but primarily serve to covalently connect a conjugate moiety to an oligonucleotide or biocleavable linker. Such linkers may comprise a chain structure or an oligomer of repeating units such as ethylene glycol, amino acid units or amino alkyl groups. In some embodiments the linker (region Y) is an amino alkyl, such as a C₂-C₃₆ amino alkyl group, including, for example C₆ to C₁₂ amino alkyl groups. In some embodiments the linker (region Y) is a C₆ amino alkyl group. Conjugate linker groups may be routinely attached to an oligonucleotide via use of an amino modified oligonucleotide, and an activated ester group on the conjugate group.

Quality Assurance Applications

The invention provides for a method for determining the sequence heterogeneity in a population of modified oligonucleotides from the same modified oligonucleotide synthesis run, said method comprising the steps of:
Obtaining or synthesizing the modified oligonucleotide,
Performing the primer based sequencing method according to the invention
Analyze the sequence data obtained to identify the sequence heterogeneity of the population of modified oligonucleotides.

The sequence heterogeneity refers to identification of the sequences of the individual species within the population, such as species which form at least 0.01%, such as at least 0.05%, such as at least 0.1%, such as at least 0.5% of the population, and based on the occurrence of each sequence optionally the proportion of the total population formed by each identified species (unique sequence).

The invention provides for a method for the validating the sequence of a modified oligonucleotide, said method comprising the steps of:
Obtaining or synthesizing the modified oligonucleotide
Performing the primer based sequencing method according to the invention
Analyse the sequence data obtained to validate the sequence of the modified oligonucleotide.

The invention provides for a method for the validating the predominant sequence within a population of modified oligonucleotides, said method comprising the steps of:
Obtaining or synthesizing the modified oligonucleotide
Performing the primer based sequencing method according to the invention
Analyse the sequence data obtained to validate the sequence of the modified oligonucleotide.

The population of modified oligonucleotides may, for example, originate from the same sequencing run (Batch) or a pool of sequencing runs (Batches).

The validation may be used to identify incorrect input errors into the modified oligonucleotide synthesis step, which may for example, result from a typographical error, or an error or contaminant used in the synthesis method step. Alternatively, the validation may be used to confirm the identity of a modified oligonucleotide, e.g. the modified oligonucleotide may be obtained from a patient who has been administered the modified oligonucleotide (e.g. in the form of a therapeutic). Sequence validation may identify incorrect sequences or truncated oligonucleotides or prolonged oligonucleotides or aberrant synthesis products.

The invention provides for a method for the determination of the purity of a modified oligonucleotide
Obtaining or synthesizing the modified oligonucleotide
Performing the primer based sequencing method according to the invention
Analyse the sequence data obtained to determine the purity of the modified oligonucleotide.

The invention provides for the use of massively parallel sequencing to sequence the nucleobase sequence of a population of modified oligonucleotides, such as phosphorothioate oligonucleotides or sugar modified oligonucleotides, such as sugar modified phosphorothioate oligonucleotides, such as phosphorothioate oligonucleotides comprising LNA and/or 2′-O-methoxyethyl modified nucleosides.

The invention provides for the use of massively parallel sequencing to sequence the nucleobase sequence of a therapeutic oligonucleotide, such as a population of therapeutic modified oligonucleotides, such as phosphorothioate oligonucleotides or sugar modified oligonucleotides, such as sugar modified phosphorothioate oligonucleotides, such as phosphorothioate oligonucleotides comprising LNA and/or 2′-O-methoxyethyl modified nucleosides.

The invention provides for the use of sequencing by synthesis sequencing to sequence the nucleobase sequence of a population of modified oligonucleotides, such as phosphorothioate oligonucleotides or sugar modified oligonucleotides, such as sugar modified phosphorothioate oligonucleotides, such as phosphorothioate oligonucleotides comprising LNA and/or 2′-O-methoxyethyl modified nucleosides.

The invention provides for the use of primer based polymerase sequencing to determine the quality of the product of a synthesis or manufacturing run of a modified oligonucleotide, such as phosphorothioate oligonucleotide or sugar modified oligonucleotide, such as sugar modified phosphorothioate oligonucleotide, such as phosphorothioate oligonucleotide comprising LNA and/or 2′-O-methoxyethyl modified nucleosides.

The invention provides for the use of primer based polymerase sequencing to determine the heterogeneity of the product of a synthesis or manufacturing run of a modified oligonucleotide, such as phosphorothioate oligonucleotide or sugar modified oligonucleotide, such as sugar modified phosphorothioate oligonucleotide, such as phosphorothioate oligonucleotide comprising LNA and/or 2′-O-methoxyethyl modified nucleosides.

The invention provides for the use of massively parallel sequencing to determine the quality of the product of a synthesis or manufacturing run of a modified oligonucleotide, such as phosphorothioate oligonucleotide or sugar modified oligonucleotide, such as sugar modified phosphorothioate oligonucleotide, such as phosphorothioate oligonucleotide comprising LNA and/or 2′-O-methoxyethyl modified nucleosides.

The invention provides for the use of sequencing by synthesis to determine the heterogeneity of the product of a synthesis or manufacturing run of a modified oligonucleotide, such as phosphorothioate oligonucleotide or sugar modified oligonucleotide, such as sugar modified phosphorothioate oligonucleotide, such as phosphorothioate oligonucleotide comprising LNA and/or 2′-O-methoxyethyl modified nucleosides.

The invention provides for the use of sequencing by synthesis to determine the quality of the product of a synthesis or manufacturing run of a modified oligonucleotide, such as phosphorothioate oligonucleotide or sugar modified oligonucleotide, such as sugar modified phosphorothioate oligonucleotide, such as phosphorothioate oligonucleotide comprising LNA and/or 2′-O-methoxyethyl modified nucleosides.

The invention provides for the use of sequencing by synthesis to determine the heterogeneity of the product of a synthesis or manufacturing run of a modified oligonucleotide, such as phosphorothioate oligonucleotide or sugar modified oligonucleotide, such as sugar modified phosphorothioate oligonucleotide, such as phosphorothioate oligonucleotide comprising LNA and/or 2′-O-methoxyethyl modified nucleosides.

In some embodiments, the method of the invention is for determining the degree of purity or heterogeneity in the population of modified oligonucleotides, e.g. a single oligonucleotide synthesis batch or a pool of multiple oligonucleotide synthesis batches.

In some embodiments, the method of the invention is for determining the sequence of the modified oligonucleotide, or the predominant sequences present in the population of modified oligonucleotides, e.g. a modified oligonucleotide synthesis batch or a pool of multiple oligonucleotide synthesis batches.

In Vivo & Drug Discovery Applications

Whilst for many years oligonucleotide therapeutics has provided the promise of going from target sequence to drug designed by Watson-Crick base pairing rules, in practice this has been very difficult to achieve and it has recently become apparent that individual sequences of oligonucleotides may have a profound effect on the pharmacological distribution of an oligonucleotide. It is therefore difficult to presume that a compound which has been select on the basis of its outstanding effect in vitro will have the same outstanding effect in vivo—simply put, its biodistribution may result in accumulation in non-target tissues and a low pharmacological effect in the target tissue. The present invention provides a method of parallel sequencing LNA or 2′-O-MOE modified oligonucleotides, allowing the identification of the cryptic sequences which result in the uptake in the desired tissues, and/or avoid accumulation in non-target tissues. This may be achieved by making libraries of oligonucleotides with different sequences (e.g. degenerate oligonucleotide libraries) and using them in a method for identifying a nucleoside modified oligonucleotide (sequence) which is enriched in a target tissue in a mammal.

The invention provides for a method for identifying a modified oligonucleotide (sequence) which is enriched in a target tissue in a mammal said method comprising:
a. Administering the mixture of modified oligonucleotides to a mammal, wherein each member of the mixture of modified oligonucleotides comprises a unique nucleobase sequence,
b. Allow for the modified oligonucleotides to be distributed within the mammal, for example for a period of at least 12 hours, such as 12-96 hours, such as 24-48 hours;
c. Isolate a population of modified oligonucleotides from one or more tissues or cells from the mammal, including a desired target tissue or desired target cell,
d. Perform the method according to the invention, including the step of sequencing the population of modified oligonucleotides, to
e. Identify a modified oligonucleotide sequences which are enriched in the desired target tissue or cell of the mammal.

The modified oligonucleotide comprises a region of nucleobases which are unique (the unique nucleobase sequence). The region may be a region of degenerate nucleobases, e.g. for the discovery of aptamers or aptameric sequences, or may be a region of known sequence, for example a molecular bar-code region (e.g. for conjugate moiety discovery).

Alternatively or in combination, the method may be used to identify a modified oligonucleotide (sequence) which has low accumulation in a non-target tissue in a mammal, said method comprising:
a. Administering the mixture of modified oligonucleotides to a mammal, wherein each member of the mixture of modified oligonucleotides comprises a unique nucleobase sequence,
b. Allow for the nucleoside modified oligonucleotides to be distributed within the mammal, for example for a period of at least 12 hours, such as 12-96 hours, such as 24-48 hours;
c. Isolate a population of modified oligonucleotides from one or more target tissues or cells from of the mammal, including a non-target tissue or non-target cell, and
d. Perform the method according to the invention, including the step of sequencing the population of modified oligonucleotides, to
e. Identify a modified oligonucleotide sequences which have a low accumulation in the non-target tissue or non-target cell of the mammal.

It will be understood that the above methods may be combined within a single in vivo experiment.

The methods may be used to identify modified oligonucleotides which have a desired biodistribution or tissue uptake profile (self-targeting), or may be used to identify oligonucleotides sequences which can target modified oligonucleotides (i.e. modified oligonucleotides which comprise “aptameric” targeting sequences). Alternatively, as described herein, the methods may be used to identify conjugate moieties which can be used to target modified oligonucleotides, thereby enhancing the biodistribution or tissue uptake profile.

It is further envisaged that in vitro assays for tissue/cellular uptake may be employed in place of, or in addition to the in vivo assays.

The invention provides a method for identifying a modified oligonucleotide or modified oligonucleotide sequence which has enhanced cellular uptake said method comprising:
a. Administering a mixture of modified oligonucleotides wherein each member of the mixture of modified oligonucleotides comprises a unique nucleobase sequence, to a cell or a population of cells for a period of time, for example 1-36 hours;
b. Isolate a population of modified oligonucleotides from the cell or population of cells,
c. Perform the method according to the invention, including the step of sequencing the population of modified oligonucleotides obtained in b; to
d. Identify one or more modified oligonucleotide sequences which are enriched in the cell or in the population of cells.

The cell may be a mammalian cell, such as a rodent cell, such as a mouse or rat cell, or may be a primate cell, such as a monkey cell or a human cell. It is also envisaged that the cell may be a pathogen cell, such as a bacterial cell or a parasitic cell, e.g a malaria cell.

After step a., and prior to step b. the cells may be washed to remove any oligonucleotides present in the cell media or attached to the external surface of the cells.

For in vitro administration, the administering step may be performed using gymnosis (unassisted uptake).

Typically, the step of isolation of the nucleoside modified oligonucleotides from the sample (e.g. obtain from the cells/population of cells, or in the case of the in vivo embodiment, from tissue or cells obtained from the mammal (or patient) is RNase treated and may be further purified (e.g. via gel or column purification) prior to use in the oligonucleotide capture method of the invention. The in vitro or in vivo methods of the invention may further comprise sub-cellular fractionation of target cells to identify modified oligonucleotides which accumulate in defined sub-cellular compartment.

The invention provides a method for identifying a modified oligonucleotide or modified oligonucleotide sequence which has a desired sub-cellular compartmentalization, said method comprising:
a. Administering a mixture of modified oligonucleotides wherein each member of the mixture of modified oligonucleotides comprises a unique nucleobase sequence, to a cell or a population of cells for a period of time, for example 1-36 hours;
b. Performing sub-cellular fractionation on the cells;
c. Isolate a population of modified oligonucleotides from at least one sub-cellular fraction, or a range of different sub-cellular fractions;
d. Perform the method according to the invention, including the step of sequencing the population of modified oligonucleotides obtained in c; to
e. Identify one or more modified oligonucleotide sequences which are enriched in one or more desired sub-cellular fractions.

The methods of the invention may be applied to identified nucleic acid sequences with aptameric properties, for example nucleic acid sequences which have a desired biodistribution in vivo. The method of the invention may therefore be used to identify aptameric sequences which can target drug molecules to the desired site of action/tissue.

In some embodiments the modified oligonucleotide is or comprises a region of 10-60 degenerate nucleotides, such as phosphorothioate linked nucleotides. In some embodiments the nucleotides of the degenerated nucleotide region are DNA nucleotides. The mixture of modified oligonucleotides with different nucleobase sequences may therefore comprises a region of 10-60 degenerate nucleotides. In some embodiments the modified oligonucleotide comprises a 5′ region (X) of defined (known) sequence, and a 3′ region (Y) which is the region of degenerate sequences, for example a region of 10-60 degenerate nucleotides. The 5′ region may for example be a the 2′ sugar modified oligonucleotide as described herein and may be a therapeutic oligonucleotide (i.e. the drug cargo). Optionally a further 3′ terminal region (Z) may be used which comprise a region of known nucleotides. Region Z may facilitate the ligation to the 3′ capture probe. The modified oligonucleotide may therefore be of formula [region X-region Y-region Z], or [region X-region-Y], or [region Y-region Z], or in some embodiments [region Y]. Once an aptameric sequence with the desired properties has been identified, it may be covalently attached to a drug molecule, for example a therapeutic oligonucleotide, such as a siRNA or an antisense oligonucleotide. In some embodiments the modified oligonucleotide comprises or consists of regions X-Y, or regions X-Y-Z. In some embodiments the modified oligonucleotide is a phosphorothioate oligonucleotide. In some embodiments the internucleoside linkages within region Y are phosphorothioate internucleoside linkages. In some embodiments the nucleosides in region Y are DNA nucleosides.

In some embodiments, the aptameric region of the modified oligonucleotide may comprise one or more stereodefined phosphorothioate internucleoside linkages. In this respect the modified oligonucleotide may comprise a region or regions of nucleosides which are linked by stereodefined internucleoside linkages, such as stereodefined phosphorothioate internucleoside linkages, and may further comprise regions which are not stereodefined. In some embodiments region Y comprises or is a region of nucleosides which are linked by stereodefined phosphorothioate internucleoside linkages. An advantage of this approach to identify stereodefined aptameric sequences is that the modified oligonucleotides may further comprise molecule bar coding regions which allow the molecular identification of molecules with a unique stereodefined internucleoside linkage motif (the bar code being associated with a specific stereodefined internucleoside linkage motif). In this respect the mixture of modified oligonucleotides may be a library of modified oligonucleotides each comprising a unique stereodefined internucleoside linkage motif and a molecular bar code. It is therefore considered that the diversity in aptameric sequences between modified oligonucleotides may be created by the pattern of stereodefined internucleoside linkage motifs, rather than or in addition to the diversity in nucleotide sequence.

Targeting Conjugate Discovery: The above discovery methods may be used for the identification of conjugate moieties which target drug cargos to the desired target tissue or cell. In some embodiments, the drug cargo may be a modified oligonucleotides, including siRNAs and antisense oligonucleotides, such as a phosphorothioate oligonucleotide, a LNA oligonucleotide or a 2′-O-methoxyethyl oligonucleotide, the method can be used to identify conjugates which may be subsequently attached to other drug modalities. In this respect the modified oligonucleotide may be a therapeutic oligonucleotide (or a potential therapeutic oligonucleotide), or it may be a tool oligonucleotide used to identify conjugates for subsequent use with other drug cargos.

The method involves the step of conjugating a library of conjugate moieties to a series of bar coded modified oligonucleotides (i.e. the modified oligonucleotide comprises a known sequence), so that each conjugate moiety can be identified by the sequencing of the bar coded modified oligonucleotide to which it is covalently attached (conjugated to). The library of bar coded modified oligonucleotide conjugates is then administered to an organism or a cell, e.g. a mammal, or a cell, and after a suitable period of time (e.g. see the above methods), the population of coded modified oligonucleotide conjugates is isolated from the cells or selected cells, or tissues from organism, and the modified oligonucleotides are sequenced using parallel sequencing methods, e.g. as according to the methods described herein. The identification of the unique bar codes, and in some embodiments the frequency of a bar code sequence being identified can then be used to identify the conjugate moieties which provide enhanced uptake into the cells, such as the selected cells, or tissues from the organism.

In some embodiments, for example for conjugate moiety discovery applications the modified oligonucleotides within the population comprises a 5′ region (X′) which is defined sequence—i.e. is a common sequence within the population of modified oligonucleotides, which may comprise a primer binding site for PCR and/or clonal amplification, and a region positioned 3′ to the 5′ region which comprises a molecular bar code region (Y′).

In some embodiments the modified oligonucleotide may comprise a further region (Z′), which comprises a further common sequence shared by the population of modified oligonucleotides, which may comprise a primer binding site for PCR and/or clonal amplification. The inclusion of a 3′ primer binding site region allows for the direct PCR amplification or clonal amplification, and parallel sequencing without the ligation of a 3′ capture probe. Alternatively, a 3′ capture probe ligation may be used. Suitably a library of different conjugate moieties are conjugated to the 5′ region of the modified oligonucleotide, optionally via a linker and/or cleavable linker. For conjugate discovery, the modified oligonucleotide may comprise a region of 3′ terminal 2′ sugar modified nucleotides, for example a region of one or more 2′-O-MOE nucleosides (e.g. a region comprising 1, 2, 3, 4 or 4 2′-O-MOEs) which protect the regions X′ and/or Z′ from exonuclease cleavage. The modified oligonucleotide may be a phosphorothioate oligonucleotide. The modified oligonucleotide may be part of an siRNA complex or may comprise an antisense oligonucleotide.

The invention provides for a method for identifying a modified oligonucleotide or modified oligonucleotide sequence which has enhanced cellular uptake in a cell said method comprising:
i. Administering a population of modified oligonucleotides wherein each member of the population of modified oligonucleotides comprises a unique nucleobase sequence to the cell;
ii. After a period of time, isolate the modified oligonucleotides from within the cell(s),
iii. Perform the method according to any one of embodiments 2-43, to parallel sequence the modified oligonucleotides obtained in step (ii); to
iv. Identify one or more modified oligonucleotide sequences which are enriched in the cell or in the population of cells.

The method may be in vitro or in vivo.

The invention provides for a method for identifying a modified oligonucleotide (sequence) which is enriched in a target tissue or cell in a mammal said method comprising:
i. Administering the mixture of modified oligonucleotides to a mammal, wherein each member of the mixture of modified oligonucleotides comprises a unique nucleobase sequence,
ii. Allow for the modified oligonucleotides to be distributed within the mammal, for example for a period of at least 6 hours;
iii. Isolate a population of modified oligonucleotides from one or more tissues or cells from the mammal, including a desired target tissue or desired target cell,
iv. Perform the method according to any one of embodiments 2-38, including the step of parallel sequencing the population of modified oligonucleotides, to
v. Identify the modified oligonucleotide sequences which are enriched in the desired target tissue or cell of the mammal.

In some embodiments, each member of the population of the modified oligonucleotides comprises a different (unique) molecular bar-code sequence.

In some embodiments, each member of the population of modified oligonucleotides comprises a different aptameric sequence.

In some embodiments, each member of the population of modified oligonucleotides comprises a different conjugate moiety.

In some embodiments, said method is to identify aptamers or aptameric sequences which are preferentially taken up by the cell, such as a target cell or target tissue.

In some embodiments, said method is to identify conjugate moieties which are preferentially taken up by the cell, such as a target cell or target tissue.

Nano Particle Targeting Discovery: The methods of the invention may be used for the identification of nanoparticles which can target drug cargos to the desired target tissue or cell.

The method involves the steps of formulating a series of unique bar-coded oligonucleotides into a range of nanoparticle formulations so that each nanoparticle formulation contains a unique bar-coded oligonucleotide (i.e. the oligonucleotide comprises a known “bar coding” sequence which can be used to identify the nanoparticle), so that each nanoparticle can be identified by the sequencing of the bar coded oligonucleotide to which it comprises. The bar coded oligonucleotide may be within the nanoparticle (e.g. a cargo molecule) or it may form part of the nanoparticle formulation (e.g. via a lipophilic conjugate which is integrated into a lipid lay on the surface of the nanoparticle).

Once formulated, the populations of oligonucleotide bar coded nanoparticles may be pooled to provide a library of bar coded oligonucleotide nanoparticles which are then administered to a cell, a tissue or an organism (such as a mammal).

The parallel sequencing methods of the invention may then be used to determine the level of nanoparticle delivery into a target cell or target tissue. Suitably this is achieved by isolating the oligonucleotide from the target cell or tissue, and performing the parallel sequencing method of the invention to determine the content of each unique bar-coded oligonucleotide, and thereby the efficacy of delivery of each nanoparticle formulation.

Alternatively stated, in a method of the invention a library (or population) of unique bar coded modified oligonucleotide nanoparticles is administered to an organism or a cell, e.g. a mammal, or a cell, and after a suitable period of time (e.g. see the above methods), the population of coded modified oligonucleotide is isolated from the cells or selected cells, or tissues from organism, and the modified oligonucleotides are sequenced using parallel sequencing methods, e.g. as according to the methods described herein. The identification of the unique bar codes, and in some embodiments the frequency of a bar code sequence being identified can then be used to identify the nanoparticles which provide enhanced uptake into the cells, such as the selected cells, or tissues from the organism.

A nanoparticle which is successfully targeted to a target cell or tissue may then be used to target a drug cargo.

In some embodiments the bar-coded oligonucleotide(s) are modified oligonucleotides, such as 2′ sugar modified oligonucleotides, such as LNA or 2′-O-MOE modified oligonucleotides.

In some embodiments the bar-coded oligonucleotide(s) are phosphorothioate oligonucleotides.

In some embodiments, the drug cargo may be a modified oligonucleotides, including siRNAs and antisense oligonucleotides, such as a phosphorothioate oligonucleotide, a LNA oligonucleotide or a 2′-O-methoxyethyl oligonucleotide, the method can be used to identify nanoparticles which may be subsequently attached to other drug modalities. In this respect the modified oligonucleotide may be a therapeutic oligonucleotide (or a potential therapeutic oligonucleotide), or it may be a tool oligonucleotide used to identify nanoparticles for subsequent use with other drug cargos.

In some embodiments, for example for nanoparticle discovery applications the unique bar coded oligonucleotides comprises a 5′ region (X′) which is defined sequence—i.e. is a common sequence within the population of modified oligonucleotides, which may comprise a primer binding site for PCR and/or clonal amplification, and a region positioned 3′ to the 5′ region which comprises a molecular bar code region (Y′).

In some embodiments the modified oligonucleotide may comprise a further region (Z′), which comprises a further common sequence shared by the population of modified oligonucleotides, which may comprise a primer binding site for PCR and/or clonal amplification. The inclusion of a 3′ primer binding site region allows for the direct PCR amplification or clonal amplification, and parallel sequencing without the ligation of a 3′ capture probe. Alternatively, a 3′ capture probe ligation may be used. Suitably a library of different conjugate moieties are conjugated to the 5′ region of the modified oligonucleotide, optionally via a linker and/or cleavable linker. For nanoparticle discovery, the modified oligonucleotide may comprise a region of 3′ terminal 2′ sugar modified nucleotides, for example a region of one or more 2′-O-MOE nucleosides (e.g. a region comprising 1, 2, 3, 4 or 4 2′-O-MOEs) which protect the regions X′ and/or Z′ from exonuclease cleavage. The modified oligonucleotide may be a phosphorothioate oligonucleotide. The modified oligonucleotide may be part of an siRNA complex or may comprise an antisense oligonucleotide.

The invention provides for a method for identifying a nanoparticle comprising a modified oligonucleotide sequence, which has enhanced cellular uptake in a cell said method comprising:
i. Administering a population of nanoparticles, each comprising a modified oligonucleotides comprising a unique nucleobase sequence (bar code) to the cell;
ii. After a period of time, isolate the modified oligonucleotides from within the cell(s),
iii. Perform the method according to any one of embodiments or claims as described herein, to parallel sequence the modified oligonucleotides obtained in step (ii); to
iv. Identify one or more nanoparticle formulations which are enriched in the cell or in the population of cells.

The method may be in vitro or in vivo.

The invention provides for a method for identifying one or more nanoparticle formulation which is enriched in a target tissue or cell in a mammal said method comprising:
i. Administering the mixture of modified oligonucleotides to a mammal, wherein each member of the mixture of modified oligonucleotides comprises a unique nucleobase sequence, wherein each member of the mixture of modified oligonucleotides is formulated in a different nanoparticle formulation;
ii. Allow for the nanoparticle formulated modified oligonucleotides to be distributed within the mammal, for example for a period of at least 6 hours;
iii. Isolate a population of modified oligonucleotides from one or more tissues or cells from the mammal, including a desired target tissue or desired target cell,
iv. Perform the method according to any one of claims or embodiments as described herein, including the step of parallel sequencing the population of modified oligonucleotides, to
v. Identify the modified oligonucleotide sequences which are enriched in the desired target tissue or cell of the mammal, and thereby identifying one or more nanoparticle formulations which are targeted to the target tissue or cell.

In some embodiments, the method or use of the invention is to identify nanoparticles which are preferentially taken up by the cell, such as a target cell or target tissue.

EMBODIMENTS—the following exemplary embodiments of the invention may be combined with other aspects or embodiments of the invention as described or claimed herein.
1. A method for sequencing the nucleobase sequence of a modified oligonucleotide, such as an 2′sugar modified oligonucleotide, such as a LNA or 2′-O-methoxyethyl modified phosphorothioate oligonucleotide, said method comprising the steps of:
   I. Perform polymerase mediated 5′-3′ first strand synthesis from the 2′ sugar modified oligonucleotide to produce a nucleic acid sequence comprising the complement of the 2′ sugar modified oligonucleotide;
   II. Perform primer based sequencing of the first strand synthesis product obtained in step II.
2. The method according to embodiment 1, wherein after step I and prior to step II the first strand product of step I is PCR amplified.
3. The method according to embodiment 1 or 2, wherein step II comprises the clonal amplification of the first strand synthesis products or PCR amplification products, prior to the primer based sequencing.
4. The method according to any one of embodiments 1-3, wherein the method is for the parallel sequencing of multiple modified oligonucleotide species within a population of modified oligonucleotides species present in a modified oligonucleotide sample.
5. The method according to any one of embodiments 1-5, wherein prior to the polymerase mediated 5′-3′ first strand synthesis, a first primer which is complementary to a 3′ region of the modified oligonucleotide is hybridized to the modified oligonucleotide for initiation of the 5′-3′ first strand synthesis.
6. The method according to any one of embodiments 1-5, wherein prior to the polymerase mediated 5′-3′ first strand synthesis, the modified oligonucleotide is ligated to a 3′ capture probe which is either a self-priming capture probe or comprises a first primer binding site.
7. The method according to embodiment 6, wherein prior to the first strand synthesis step a first primer is hybridized to the 3′ capture probe for initiation of the polymerase mediated first strand synthesis.
8. The method according to embodiment 6 or 7, wherein after ligation of the 3′ capture probe to the modified oligonucleotide, and prior to first strand synthesis the ligation product is purified e.g. via gel purification, or via enzymatic degradation of the un-ligated 3′ capture probe.
9. The method according to any one of embodiments 6-8, wherein the PCR step is performed using a PCR primer pair, wherein one of the PCR primers is specific for the 3′ capture probe, and the second PCR primer is specific for the modified oligonucleotide, such as a 5′ region of the modified oligonucleotide.
10. The method according to any one of embodiments 6-9, wherein the sequencing step II comprises the clonal amplification of the first strand product of step I using a clonal amplification primers, wherein one of the clonal amplification primers is specific for the 3′ capture probe, and the second clonal amplification primer is specific for the modified oligonucleotide such as a 5′ region of the modified oligonucleotide.
11. The method according to any one of embodiments 6-8, wherein after the first strand synthesis step I, and first strand synthesis product purification if performed, an adapter probe is ligated at the 3′ end of the first strand synthesis product.
12. The method according to embodiment 11, wherein the PCR step is performed using a pair of PCR primers wherein one of the PCR primers is specific for the 3′ capture probe and the other PCR primer is specific for adapter probe.
13. The method according to embodiment 11 or 12, wherein the sequencing step II comprises the clonal amplification of the first strand synthesis product of step I using clonal amplification primers specific for the 3′ capture probe and adapter probe respectively.
14. The method according to any one of embodiments 1-12, wherein after first strand synthesis, the first strand synthesis product is polyadenylated.
15. The method according to embodiment 14, wherein a second strand is synthesized from the first strand using a poly T primer.
16. The method according to embodiment 15, wherein the polyT primer further comprises a PCR primer binding site and/or a clonal amplification primer binding site.
17. The method according to anyone of embodiments 14-16, wherein the PCR step is performed using a primer which is specific for the 3′ capture probe and either the poly T primer or a PCR primer which is specific for the PCR primer binding site in the poly T primer.
18. The method according to any one of embodiments 14-16, wherein the PCR step is performed using a primer which is specific for modified oligonucleotide, such as the 3′ region of the modified oligonucleotide, and either the poly T primer or a PCR primer which is specific for the PCR primer binding site in the poly T primer.
19. The method according to any one of embodiments 14-18, wherein the sequencing step comprises clonal amplification wherein one of the clonal amplification primers is specific for the 3′ capture probe or the modified oligonucleotide (such as 3′region of the modified oligonucleotide), and the second clonal amplification primer is specific for the poly T primer.
20. The method according to embodiment 18, wherein the PCR primers further comprise clonal amplification primer binding sites, such as flow cell capture probe binding sites, wherein the sequencing step comprises clonal amplification using clonal amplification primers, such as flow cell binding primers, which are complementary to the clonal amplification primer binding sites.
21. The method according to any one of embodiments 1-4, wherein prior to first strand synthesis wherein prior to the polymerase mediated 5′-3′ first strand synthesis, the modified oligonucleotide is ligated to a 5′ capture probe which comprises a primer binding site.
22. The method according to embodiment 21, wherein either the modified oligonucleotide comprises a 5′ phosphate group, or prior to the ligation of the 5′ capture probe the 5′ terminus of the modified oligonucleotide is phosphorylated.
23. The method according to embodiment 21 or 22, wherein the PCR step is performed using a first PCR primer specific for the modified oligonucleotide, such as the 3′ region of the modified oligonucleotide, and a second PCR primer which is specific for the 5′capture probe.
24. The method according to embodiment 21 or 22, wherein prior to first strand synthesis, a 3′ capture probe is ligated to the 3′ terminus of the modified oligonucleotide, either prior to or subsequent to or simultaneous to the ligation of the 5′ capture probe to the 5′ terminus of the modified oligonucleotide, wherein the 3′ capture probe is either a self-priming capture probe or comprises a first primer binding site.
25. The method according to any one of embodiments 21-24, wherein prior to first strand synthesis the ligation product(s) is purified e.g. via gel purification, or via enzymatic degradation of the unligated 5′, and if used 3′ capture probes.
26. The method according to embodiment 24 or 25, wherein the first strand synthesis is initiated using a first primer hybridized to the 3′ capture probe or from the self-priming 3′ capture probe.
27. The method according to any one of embodiments 24-26, wherein the PCR step is performed using a first PCR primer which is specific for the 3′ capture probe and a second PCR primer which is specific for the 5′ capture probe.
28. The method according to embodiment 23 or 27, wherein the PCR primers comprise clonal amplification primer binding sites (such as flow cell primer binding sites), and the sequencing step comprises a clonal amplification.
29. The method according to any one of embodiments 24-28, wherein the sequencing step II comprises the clonal amplification of the first strand product of step I using a clonal amplification primers, wherein one of the clonal amplification primers is specific for the 3′ capture probe, and the second clonal amplification primer is specific for the modified oligonucleotide such as a 5′ region of the modified oligonucleotide.
30. The method according to any one of embodiments 6-29, wherein the 3′ capture probe is ligated to the 3′ end of the modified oligonucleotide using T4 DNA ligase.
31. The method according to any one of embodiments 21-30, wherein the 5′ capture probe is ligated to the 5′ end of the modified oligonucleotide using T4 RNA ligase or T4 RNA ligasell.
32. The method according to any one of embodiments 1-31, wherein the sequencing step II comprises clonal amplification and the clonal amplification primers are bound to a solid support [e.g. a flow cell] or are compartmentalized within an emulsion droplet.
33. The method according to embodiment 32, wherein the solid phase amplification is solid phase bridge amplification.
34. The method according to any one of embodiments 1-32, wherein the primer based sequencing method is performed using sequencing by synthesis method.
35. The method according to any one of embodiments 1-33, wherein the primer based sequencing method is a cyclic reversible termination method (CRT).
36. The method according to any one of embodiments 1-34 wherein the sequencing is a parallel sequencing method, such as massively parallel sequencing.
37. The method according to any one of embodiments 1-35, wherein the primer based sequencing is performed using clonal bridge amplification (e.g. Illumina sequencing—reversible dye terminator), or clonal emulsion PCR (Roche 454, GS FLX Titanium, Life Technologies SOLiD4, Life Technologies Ion Proton).
38. The method according to any one of embodiments 1-37, wherein the first strand synthesis is performed in the presence of a polymerase and polyethylene glycol or propylene glycol.
39. The method according to any one of embodiments 1-38, wherein the polymerase used for first strand synthesis is Taq polymerase or Volcano2G polymerase or PrimeScript reverse transcriptase.
40. The method according to embodiment 2-39, wherein the sequencing step comprises the clonal amplification of the PCR amplification product(s) using clonal amplification primers.
41. The method according to any one of embodiments 1-40, wherein the 2′ sugar modified oligonucleotide is a 2′ sugar modified phosphorothioate oligonucleotide.
42. The method according to any one of embodiments 1-41, wherein the modified oligonucleotide comprises at least two contiguous 2′ sugar modified nucleosides.
43. The method according to any one of embodiments 1-42, wherein the modified oligonucleotide comprises at least one 2′-O-methoxyethyl RNA (MOE) nucleoside.
44. The method according to any one of embodiments 1-43, wherein the modified oligonucleotide comprises at least two contiguous 2′-O-methoxyethyl RNA (MOE) nucleosides.
45. The method according to any one of embodiments 1-44, wherein the modified oligonucleotide comprises at least one 2′-O-methoxyethyl RNA (MOE) nucleoside located at the 3′ of the modified oligonucleotide, such as at least two or at least three contiguous 2′-O-methoxyethyl RNA (MOE) nucleosides located at the 3′ end of the modified oligonucleotide.
46. The method according to any one of embodiments 1-45, wherein the modified oligonucleotide comprises at least 1 LNA nucleoside.
47. The method according to any one of embodiments 1-46, wherein the modified oligonucleotide comprises at least two contiguous LNA nucleotides or at least three contiguous LNA nucleotides.
48. The method according to embodiment 1-47, wherein the modified oligonucleotide comprises one or more LNA nucleotide(s) are located at the 3′ end.
49. The method according to any one of embodiments 1-48, wherein the modified oligonucleotide is a LNA phosphorothioate oligonucleotide.
50. The method according to any one of embodiments 1-49, wherein the modified oligonucleotide comprises both LNA nucleosides and DNA nucleosides, such as a LNA gapmer, or LNA mixmer.
51. The method according to any one of embodiments 1-50, wherein the LNA oligonucleotide comprises at least one LNA-T nucleoside or at least one LNA-C nucleoside.
52. The method according to any one of embodiments 1-51, wherein the modified oligonucleotide comprises one or more LNA nucleoside(s) and one or more 2′substituted nucleoside, such as one or more 2′-O-methoxyethyl nucleosides.
53. The method according to any one of embodiments 1-52 wherein the modified oligonucleotide is selected from the group consisting of; a 2′-O-methoxyethyl gapmer, a mixed wing gapmer, an alternating flank gapmer or a LNA gapmer.
54. The method according to any one of embodiments 1-52, wherein the modified oligonucleotide is a mixmer or a totalmer.
55. The method according to any one of embodiments 1-54, wherein the modified oligonucleotide comprise a conjugate group, such as a GalNAc conjugate.
56. The method according to any one of embodiments 1-55, wherein said method is for determining the degree of purity or heterogeneity in the population of modified oligonucleotides, e.g. a single oligonucleotide synthesis batch or a pool of multiple oligonucleotide synthesis batches.
57. The method according to any one of embodiments 1-56, wherein said method is for determining the sequence of the modified oligonucleotide, or the predominant sequences present in the population of modified oligonucleotides, e.g. a modified oligonucleotide synthesis batch or a pool of multiple oligonucleotide synthesis batches.
58. The method according to any one of embodiments 1-57, wherein the modified oligonucleotide is a population of modified oligonucleotides, e.g. a population of modified oligonucelotides from the same oligonucleotide synthesis run [or batch] or a pool of oligonucleotide synthesis runs [or batches].
59. The method according to any one of embodiments 1-52, wherein the modified oligonucleotide is a population of modified oligonucleotides isolated from a cell, or a tissue or an organism.
60. The method according to any one of embodiments 1-52, wherein the method is for identifying a modified oligonucleotide which is preferentially targeted to a target tissue or a target cell, wherein said method comprises the steps of
   (i) Administering a mixture of modified oligonucleotides to an organism, such as a mammal, wherein each member of the mixture of modified oligonucleotides comprises a unique nucleobase sequence,
   (ii) Allow for the modified oligonucleotides to be distributed within the mammal and/or taken up by the cells;
   (iii) Isolate a population of modified oligonucleotides from one or more tissues or cells from the mammal, including a desired target tissue or desired target cell,
   (iv) Perform the method according to any one of the preceding embodiments, to
   (v) Identify one or more modified oligonucleotide sequences which are enriched in the desired target tissue or cell of the mammal.
61. The method according to any one of embodiments 1-52, wherein the method is for identifying a modified oligonucleotide which is preferentially taken up by a cell, such as a target cell, wherein said method comprises the steps of:
   (i) Administering a mixture of modified oligonucleotides to a cell, wherein each member of the mixture of modified oligonucleotides comprises a unique nucleobase sequence,
   (ii) Incubate the cells for a period of time;
   (iii) Isolate a population of modified oligonucleotides from the cell,
   (iv) Perform the method according to any one of embodiments 1-59, to
   (v) Identify one or more modified oligonucleotide sequences which are enriched in the cell.
62. The method according to any one of embodiments 1-61, wherein the modified oligonucleotide comprises a population of modified oligonucleotides, wherein each member of the population of modified oligonucleotides comprises an aptameric region of nucleotides.
63. The method according to any one of embodiments 1-61, wherein the modified oligonucleotide comprises a population of modified oligonucleotides, wherein each member of the population of modified oligonucleotides comprises a different conjugate group.
64. The method according to any one of embodiments 21-63, wherein the 5 capture probe is as defined in any one of embodiments 65-76.
65. A 5′ capture probe oligonucleotide, for T4 RNA ligase or T4 RNA ligase II mediated ligation to the 5′ terminus of 5′ phosphorylated modified oligonucleotides, comprising 3′-5:
   I. a first region comprising a 3′terminal RNA nucleoside or a 3′ terminal region of RNA nucleotides with a 3′ terminal —OH group;
   II. a second region of nucleotides which comprises the complement of a primer binding site, e.g. a PCR primer binding site (or a primer site for second strand synthesis);
   III. A third region, comprising
      a. A sequence of nucleotides; or
      b. a linker region which blocks polymerase read through; or
      c. a 5′ terminal group other than 5′ phosphate.
   IV. A fourth region of nucleotides which is either
      a. covalently attached with the third region, or
      b. in the event that the third region is a 5′ terminal group other than 5′-phosphase is discontinuous with the fourth region,
   V. A fifth region of nucleotides which are complementary to a 5′ region of the modified oligonucleotide, or may optionally be a region of degenerate nucleotides;
   VI. A 5′ blocking group—i.e. a 5′ group other than 5′ phosphate. wherein the second region and the fourth region comprise complementary regions of nucleotides which are capable of forming a duplex between the second a fourth regions.
66. The 5′ capture probe of embodiment 65, wherein the first region comprises 2-20 RNA nucleotides, such as 3-10 RNA nucleotides.
67. The 5′ capture probe of embodiment 65 or 66, wherein the second region (II) comprises a sequencing primer binding site and/or a flow cell binding site.
68. The 5′ capture probe of any one of embodiments 65-67, wherein the second region (II) further comprises a molecular bar-code region.
69. The 5′ capture probe of any one of embodiments 65-68, wherein the second region (II) further comprises a sequencing reaction bar-code region.
70. The 5′ capture probe of any one of embodiments 65-69, wherein the second region (II) further comprises a sequencing primer binding region.
71. The 5′ capture probe according to any one of embodiments 65-70, which comprises a linker region III which prevents polymerase read through during first strand synthesis.
72. The 5′capture probe according to embodiment 71, wherein the linker region III comprises a non-nucleotide linker, such as a C₆₋₃₂ polyethyleneglycol linker, such as a C18 polyethyleneglycol linker or an alkyl linker, or is a region of nucleotides comprising an inverted nucleoside linkage.
73. The 5′ capture probe according to embodiments 71 wherein region III comprises or is a discontinuity resulting in a two stranded 5′ capture probe, the first strand comprising regions I and II, and a second strand comprising regions IV and V.
74. The 5′ capture probe according to any one of embodiments 65-73, wherein region V comprises a predetermined region of nucleotides which are complementary to the 5′ region of a modified oligonucleotide.
75. The 5′ capture probe according to any one of embodiments 65-73, wherein region

V comprises a degenerate sequence of nucleotides.
76. The 5′ capture probe according to any one of embodiments 65-75 wherein the 5′ terminus of region V comprises a fluorescent label such as 5′-FAM.
77. A kit for use in the capture of modified oligonucleotides, said kit comprising a 3′ capture probe and a 5′ capture probe, wherein the 5′ capture probe is as according to any one of embodiments 65-76.

Further Embodiments A (These embodiments refer to certain 3′ capture probe and adaptor probe ligation methods—the dependent embodiments may apply to the other methods of the invention described herein, such as the 5′ capture probe embodiments as described or claimed here).

The invention further provides the following embodiments:

Embodiment 1: A method for sequencing the nucleobase sequence of a 2′ sugar modified phosphorothioate modified oligonucleotide said method comprising the steps of:
a. Ligating a capture probe oligonucleotide to the 3′ terminus of the modified oligonucleotide;
b. Perform polymerase mediated 5′-3′ first strand synthesis from the capture probe to produce a nucleic acid sequence comprising the complement of the modified oligonucleotide;
c. Ligate an adapter probe to the 3′ end of the first strand synthesis product obtained in step b; and subsequently either
   Perform primer based sequencing of the ligation product obtained in step c); or
   Perform PCR amplification of the ligation product obtained in step c) and perform primer based sequencing of the PCR amplification product.

Embodiment 2: A method for parallel sequencing the base sequence of a population of 2′sugar modified phosphorothioate modified oligonucleotides said method comprising the steps of:
a. Ligating a capture probe oligonucleotide to the 3′ terminus of the modified oligonucleotides present in the population of modified oligonucleotides;
b. Perform polymerase mediated 5′-3′ first strand synthesis from the capture probe to produce a population of nucleic acid sequences, each comprising the complement of base sequence of a modified oligonucleotide present in the population of modified oligonucleotides;
c. Ligate an adapter probe to the 3′ end of the first strand synthesis products obtained in step b; and subsequently either
   Perform primer based parallel sequencing of the ligation products obtained in step c); or
   Perform PCR amplification of the ligation products obtained in step c) and perform primer based parallel sequencing of the PCR amplification products.
2. The method according to embodiment 1 or 2, wherein the capture probe comprises a first primer binding site, and prior to first strand synthesis a first primer is hybridized to the capture probe for initiation of first strand synthesis.
3. The method according to embodiment 1 or 2, wherein the capture probe is a self-priming capture probe.
4. The method according to any one of embodiments 1-4, wherein the capture probe and the adaptor probe comprise clonal amplification primer binding sites and the sequencing step comprises clonal amplification of the ligation products obtained in step c or the PCR amplification product.
5. The method according to any one of embodiments 1-5, wherein the PCR amplification step is performed using a pair of PCR primers, one which is specific for the capture probe oligonucleotide, the other which is specific for the adapter probe;
6. The method according to embodiment 6, wherein the PCR amplification primers comprise clonal amplification primer binding sites, and the primer based sequencing step comprises clonal amplification of the PCR amplification products.
7. The method according to any one of embodiments 1-5, wherein the clonal amplification primers are specific for the first and second PCR primers; or the clonal amplification primers are specific for the 3′ capture probe and adaptor probe; or one of the clonal amplification primers is specific for one of the PCR primers, and the other clonal amplification primer is specific for either the 3′capture probe or the adaptor probe respectively.
8. The method according to any one of embodiments 1-8, wherein the primer based sequencing step is performed using sequencing by synthesis method.
9. The method according to any one of embodiments 1-9, wherein the primer based sequencing method is a cyclic reversible termination method (CRT).
10. The method according to any one of embodiments 1-10, wherein the sequencing step comprises clonal amplification and the clonal amplification primers are bound to a solid support, e.g. a flow cell, or are compartmentalized within an emulsion droplet.
11. The method according to any one of embodiments 1-11, wherein the PCR step of the primer based sequencing step comprises, either
   a. solid phase amplification such as solid phase bridge amplification, or
   b. emulsion phase amplification, such as droplet PCR.
12. The method according to any one of embodiments 1-12, wherein the primer based sequencing is performed using parallel sequencing, such as massively parallel sequencing.
13. The method according to any one of embodiments 1-13, wherein the first strand synthesis is performed in the presence of a polymerase and polyethylene glycol or propylene glycol.
14. The method according to any one of embodiments 1-14, wherein the polymerase used for first strand synthesis is Taq polymerase or Volcano2G polymerase or PrimeScript reverse transcriptase, or an effective polymerase which has at least 70% identity to Taq polymerase.
15. The method according to any one of embodiments 1-15, wherein the modified oligonucleotide is a 2′ sugar modified phosphorothioate oligonucleotide, such as a LNA phosphorothioate or a 2′-O-MOE phosphorothioate oligonucelotide.
16. The method according to any one of embodiments 1-16, wherein the modified oligonucleotide comprises at least two contiguous 2′ sugar modified nucleosides.
17. The method according to any one of embodiments 1-17, wherein the modified oligonucleotide comprises at least one 2′-O-methoxyethyl RNA (MOE) nucleoside.
18. The method according to any one of embodiments 1-18, wherein the modified oligonucleotide comprises at least two contiguous 2′-O-methoxyethyl RNA (MOE) nucleosides.
19. The method according to any one of embodiments 1-19, wherein the modified oligonucleotide comprises at least one 2′-O-methoxyethyl RNA (MOE) nucleoside located at the 3′ of the modified oligonucleotide, such as at least two or at least three contiguous 2′-O-methoxyethyl RNA (MOE) nucleosides located at the 3′ end of the modified oligonucleotide.
20. The method according to any one of embodiments 1-20, wherein the modified oligonucleotide comprises at least 1 LNA nucleoside.
21. The method according to any one of embodiments 1-21, wherein the modified oligonucleotide comprises at least two contiguous LNA nucleotides or at least three contiguous LNA nucleotides.
22. The method according to embodiment 1-22 wherein the modified oligonucleotide comprises at least one LNA nucleotide, such as at least two LNA nucleotides located at the 3′ end of the LNA oligonucleotide.
23. The method according to any one of embodiments 1-23, wherein the modified oligonucleotide is a LNA phosphorothioate oligonucleotide.
24. The method according to any one of embodiments 1-24, wherein the modified oligonucleotide comprises both LNA nucleosides and DNA nucleosides, such as a LNA gapmer, or LNA mixmer.
25. The method according to any one of embodiments 1-25, wherein the modified oligonucleotide comprises at least one 2′sugar modified T nucleoside, such as a LNA-T nucleoside or at least one 2′sugar modified C nucleoside such as a LNA-C nucleoside.
26. The method according to any one of embodiments 1-26, wherein the modified oligonucleotide comprises one or more LNA nucleoside(s) and one or more 2′substituted nucleoside, such as one or more 2′-O-methoxyethyl nucleosides.
27. The method according to any one of embodiments 1-27 wherein the modified oligonucleotide is selected from the group consisting of; a 2′-O-methoxyethyl gapmer, a mixed wing gapmer, an alternating flank gapmer or a LNA gapmer.
28. The method according to any one of embodiments 1-27, wherein the modified oligonucleotide is a mixmer or a totalmer.
29. The method according to any one of embodiments 1-29, wherein the modified oligonucleotide comprise a conjugate group, such as a GalNAc conjugate.
30. The method according to any one of embodiments 1-30, wherein said method is for determining the degree of purity or heterogeneity in the population of modified oligonucleotides, e.g. a single oligonucleotide synthesis batch or a pool of multiple oligonucleotide synthesis batches.
31. The method according to any one of embodiments 1-31, wherein said method is for determining the sequence of the modified oligonucleotide, or the predominant sequences present in the population of modified oligonucleotides, e.g. a modified oligonucleotide synthesis batch or a pool of multiple oligonucleotide synthesis batches.
32. The method according to any one of embodiments 1-32, wherein the modified oligonucleotide is a population of modified oligonucleotides, e.g. a population of modified oligonucelotides from the same oligonucleotide synthesis run [or batch] or a pool of oligonucleotide synthesis runs [or batches].
33. Use of primer based sequencing to sequence the nucleobase sequence of a population of 2′ sugar modified oligonucleotides.
34. Use of primer based sequencing to determine the heterogeneity of the product of a synthesis or manufacturing run of a 2′ sugar modified oligonucleotide.
35. Use of primer based sequencing to determine the quality of the product of a synthesis or manufacturing run of a 2′ sugar modified oligonucleotide.
36. Use of parallel sequencing to sequence the nucleobase sequence of a population of 2′ sugar modified oligonucleotides.
37. Use of parallel sequencing to determine the quality of the product of a synthesis or manufacturing run of a 2′ sugar modified oligonucleotide.
38. Use of parallel sequencing to determine the heterogeneity of the product of a synthesis or manufacturing run of a 2′ sugar modified oligonucleotide.
39. Use of sequencing by synthesis sequencing to sequence the nucleobase sequence of a population of 2′ sugar modified oligonucleotides.
40. Use of sequencing by synthesis to determine the heterogeneity of the product of a synthesis or manufacturing run of a 2′ sugar modified oligonucleotide.
41. Use of sequencing by synthesis to determine the quality of the product of a synthesis or manufacturing run of a 2′ sugar modified oligonucleotide.
42. The use according to any one of embodiments 34-42, wherein the 2′ sugar modified oligonucleotide is as defined in any one of embodiments 1-33.
43. The use of a Taq polymerase, or a polymerase enzyme with at least 70% identity to SEQ ID NO 1, for first strand synthesis from a template comprising a LNA modified phosphorothioate oligonucleotide or a 2′-O-methoxyethyl modified phosphorothioate oligonucleotide.

Further Embodiments B (These embodiments refer to certain 3′capture probe aspect—the dependent embodiments may apply to the other methods of the invention described herein, such as the 5′ capture probe embodiments as described or claimed here)

Embodiment 1: A method for sequencing the nucleobase sequence of a modified oligonucleotide said method comprising the steps of:
a. Ligating a capture probe oligonucleotide to the 3′ terminus of the modified oligonucleotide;
b. Perform polymerase mediated 5′-3′ first strand synthesis from the capture probe to produce a nucleic acid sequence comprising the complement of the modified oligonucleotide;
c. Perform primer based sequencing of the first strand synthesis product obtained in step b).

Embodiment 2: A method for parallel sequencing the base sequence of a population of modified oligonucleotides said method comprising the steps of:
d. Ligating a capture probe oligonucleotide to the 3′ terminus of the modified oligonucleotides present in the population of modified oligonucleotides;
e. Perform polymerase mediated 5′-3′ first strand synthesis from the capture probe to produce a population of nucleic acid sequences, each comprising the complement of base sequence of a modified oligonucleotide present in the population of modified oligonucleotides;
f. Perform primer based parallel sequencing of the population of first strand synthesis products obtained in step b).
2. The method according to embodiment 1 or 2, wherein the capture probe comprises a first primer binding site, and prior to first strand synthesis a first primer is hybridized to the capture probe for initiation of first strand synthesis.
3. The method according to embodiment 1 or 2, wherein the capture probe is a self-priming capture probe.
4. The method according to any one of embodiments 1-4, wherein after step b, and prior to step c. the first strand synthesis product is PCR amplified.
5. The method according to any one of embodiments 1-5, wherein step c) comprises the clonal amplification of either the first strand synthesis products of step b., or the PCR amplification product of embodiment 5, prior to primer based sequencing.
6. The method according to any one of embodiments 1-6, wherein after ligation of the 3′ capture probe to the modified oligonucleotide, and prior to first strand synthesis, the ligation product is purified e.g. via gel purification, or via enzymatic degradation of the un-ligated 3′ capture probe.
7. The method according to any one of embodiments 5-7, wherein the PCR step is performed using a PCR primer pair, wherein one of the PCR primers is specific for the 3′ capture probe, and the second PCR primer is specific for the modified oligonucleotide, such as a 5′ region of the modified oligonucleotide.
8. The method according to any one of embodiments 1-8, wherein the primer based sequencing step comprises the clonal amplification of the first strand synthesis step (b) using a clonal amplification primers, wherein one of the clonal amplification primers is specific for the 3′ capture probe, and the second clonal amplification primer is specific for the modified oligonucleotide such as a 5′ region of the modified oligonucleotide.
9. The method according to any one of embodiments 1-7, wherein after the first strand synthesis step (b), or the purification step of embodiment 7, an adapter probe is ligated at the 3′ end of the first strand synthesis product.
10. The method according to embodiment 10, wherein the PCR step is performed using a pair of PCR primers wherein one of the PCR primers is specific for the 3′ capture probe and the other PCR primer is specific for adapter probe.
11. The method according to embodiment 10 or 11, wherein the capture probe and the adaptor probe comprise clonal amplification primer binding sites and the sequencing step comprises clonal amplification of the first strand synthesis/adapter probe ligation product of embodiment 10 or the PCR amplification product of embodiment 11.
12. The method according to any one of embodiments 12, wherein the clonal amplification primers are specific for the first and second PCR primers; or the clonal amplification primers are specific for the 3′ capture probe and adaptor probe; or one of the clonal amplification primers is specific for one of the PCR primers, and the other clonal amplification primer is specific for either the 3′capture probe or the adaptor probe respectively.
13. The method according to any one of embodiments 1-7, wherein after the first strand synthesis step (b) the first strand synthesis product is polynucleated (polyN) at the 3′ end, e.g. polyadenylated.
14. The method according to embodiment 14, wherein a second strand is synthesized using a primer with a complementary poly(N) sequence, e.g. a poly T primer.
15. The method according to embodiment 15, wherein the second stand synthesis primer further comprises a PCR primer binding site and/or a clonal amplification primer binding site (or flow-cell primer binding site).
16. The method according to any one of embodiments 14-16, wherein the PCR step is performed using a primer which is specific for the 3′ capture probe and either the second strand synthesis primer or a PCR primer which is specific for the second strand synthesis primer.
17. The method according to any one of embodiments 14-17, wherein the sequencing step comprises clonal amplification wherein one of the clonal amplification primers is specific for the 3′ capture probe, and the second clonal amplification primer is specific for the second strand synthesis primer.
18. The method according to embodiment 18, wherein the PCR primers further comprise clonal amplification primer binding sites, such as flow cell capture probe binding sites, wherein the sequencing step comprises clonal amplification using clonal amplification primers, such as flow cell binding primers, which are complementary to the clonal amplification primer binding sites.
19. The method according to any one of embodiments 1-19, wherein the primer based sequencing step is performed using sequencing by synthesis method.
20. The method according to any one of embodiments 1-20, wherein the primer based sequencing method is a cyclic reversible termination method (CRT).
21. The method according to any one of embodiments 1-21, wherein the sequencing step comprises clonal amplification and the clonal amplification primers are bound to a solid support, e.g. a flow cell, or are compartmentalized within an emulsion droplet.
22. The method according to embodiment 22, wherein the clonal amplification step of the primer based sequencing step comprises, either
   a. solid phase amplification such as solid phase bridge amplification, or
   b. emulsion phase amplification, such as droplet PCR.
23. The method according to any one of embodiments 1-23, wherein the primer based sequencing is performed using parallel sequencing, such as massively parallel sequencing.
24. The method according to any one of embodiments 1-24, wherein the first strand synthesis is performed in the presence of a polymerase and polyethylene glycol or propylene glycol.
25. The method according to any one of embodiments 1-25, wherein the polymerase used for first strand synthesis is Taq polymerase or Volcano2G polymerase or PrimeScript reverse transcriptase, or an effective polymerase which has at least 70% identity to Taq polymerase.
26. The method according to any one of embodiments 1-26, wherein the modified oligonucleotide is a 2′ sugar modified phosphorothioate oligonucleotide, such as a LNA phosphorothioate or a 2′-O-MOE phosphorothioate oligonucelotide.
27. The method according to any one of embodiments 1-27, wherein the modified oligonucleotide comprises at least two contiguous 2′ sugar modified nucleosides.
28. The method according to any one of embodiments 1-28, wherein the modified oligonucleotide comprises at least one 2′-O-methoxyethyl RNA (MOE) nucleoside.
29. The method according to any one of embodiments 1-29, wherein the modified oligonucleotide comprises at least two contiguous 2′-O-methoxyethyl RNA (MOE) nucleosides.
30. The method according to any one of embodiments 1-30, wherein the modified oligonucleotide comprises at least one 2′-O-methoxyethyl RNA (MOE) nucleoside located at the 3′ of the modified oligonucleotide, such as at least two or at least three contiguous 2′-O-methoxyethyl RNA (MOE) nucleosides located at the 3′ end of the modified oligonucleotide.
31. The method according to any one of embodiments 1-31, wherein the modified oligonucleotide comprises at least 1 LNA nucleoside.
32. The method according to any one of embodiments 1-32, wherein the modified oligonucleotide comprises at least two contiguous LNA nucleotides or at least three contiguous LNA nucleotides.
33. The method according to embodiment 1-33 wherein the modified oligonucleotide comprises at least one LNA nucleotide, such as at least two LNA nucleotides located at the 3′ end of the LNA oligonucleotide.
34. The method according to any one of embodiments 1-34, wherein the modified oligonucleotide is a LNA phosphorothioate oligonucleotide.
35. The method according to any one of embodiments 1-35, wherein the modified oligonucleotide comprises both LNA nucleosides and DNA nucleosides, such as a LNA gapmer, or LNA mixmer.
36. The method according to any one of embodiments 1-36, wherein the modified oligonucleotide comprises at least one 2′sugar modified T nucleoside, such as a LNA-T nucleoside or at least one 2′sugar modified C nucleoside such as a LNA-C nucleoside.
37. The method according to any one of embodiments 1-37, wherein the modified oligonucleotide comprises one or more LNA nucleoside(s) and one or more 2′substituted nucleoside, such as one or more 2′-O-methoxyethyl nucleosides.
38. The method according to any one of embodiments 1-38 wherein the modified oligonucleotide is selected from the group consisting of; a 2′-O-methoxyethyl gapmer, a mixed wing gapmer, an alternating flank gapmer or a LNA gapmer.
39. The method according to any one of embodiments 1-39, wherein the modified oligonucleotide is a mixmer or a totalmer.
40. The method according to any one of embodiments 1-40, wherein the modified oligonucleotide comprise a conjugate group, such as a GalNAc conjugate.
41. The method according to any one of embodiments 1-41, wherein the modified oligonucleotide is an aptamer or comprises an aptameric sequence.
42. The method according to any one of embodiments 1-42, wherein the modified oligonucleotide comprises a population of modified oligonucleotide conjugates, wherein each member of the population of modified oligonucleotide conjugates comprises a different conjugate group.
43. A method for identifying a modified oligonucleotide or modified oligonucleotide sequence which has enhanced cellular uptake in a cell said method comprising:
   v. Administering a population of modified oligonucleotides wherein each member of the population of modified oligonucleotides comprises a unique nucleobase sequence to the cell;
   vi. After a period of time, isolate the modified oligonucleotides from within the cell(s),
   vii. Perform the method according to any one of embodiments 2-43, to parallel sequence the modified oligonucleotides obtained in step (ii); to
   viii. Identify one or more modified oligonucleotide sequences which are enriched in the cell or in the population of cells.
44. The method according to any one of embodiments 44, wherein the cells are in vitro.
45. The method according to any one of embodiments 44, wherein the cells are in vivo.
46. A method for identifying a modified oligonucleotide (sequence) which is enriched in a target tissue or cell in a mammal said method comprising:
   vi. Administering the mixture of modified oligonucleotides to a mammal, wherein each member of the mixture of modified oligonucleotides comprises a unique nucleobase sequence,
   vii. Allow for the modified oligonucleotides to be distributed within the mammal, for example for a period of at least 6 hours;
   viii. Isolate a population of modified oligonucleotides from one or more tissues or cells from the mammal, including a desired target tissue or desired target cell,
   ix. Perform the method according to any one of embodiments 2-38, including the step of parallel sequencing the population of modified oligonucleotides, to
   x. Identify the modified oligonucleotide sequences which are enriched in the desired target tissue or cell of the mammal.
47. The method according to any one of embodiments 44-47, wherein the each member of the population of the modified oligonucleotides comprises a different (unique) molecular bar-code sequence.
48. The method according to embodiment 44-48, wherein each member of the population of modified oligonucleotides comprises a different aptameric sequence.
49. The method according to embodiment 44-48, wherein each member of the population of modified oligonucleotides comprises a different conjugate moiety.
50. The method according to embodiment 49, wherein said method is to identify aptamers or aptameric sequences which are preferentially taken up by the cell, such as a target cell or target tissue.
51. The method according to embodiment 50, wherein said method is to identify conjugate moieties which are preferentially taken up by the cell, such as a target cell or target tissue.

### EXAMPLES

In order to be able to sequence nucleoside modified oligonucleotides, such as LNA oligonucleotides, we need a polymerase which is able to efficiently read across the entire LNA oligonucleotide. We identified that only certain polymerases are able to do this, and for some polymerases the efficacy of read through across a nucleoside modified oligonucleotide is enhanced by the presence of certain additives. Here we identify preferred polymerases and we have discovered additives for the PCR reactions that enable the polymerase to read across a test LNA oligo nucleotide.

### Example 1: Generation of Test Molecule to Test Polymerase Reading Efficiency of LNA Oligo Nucleotide

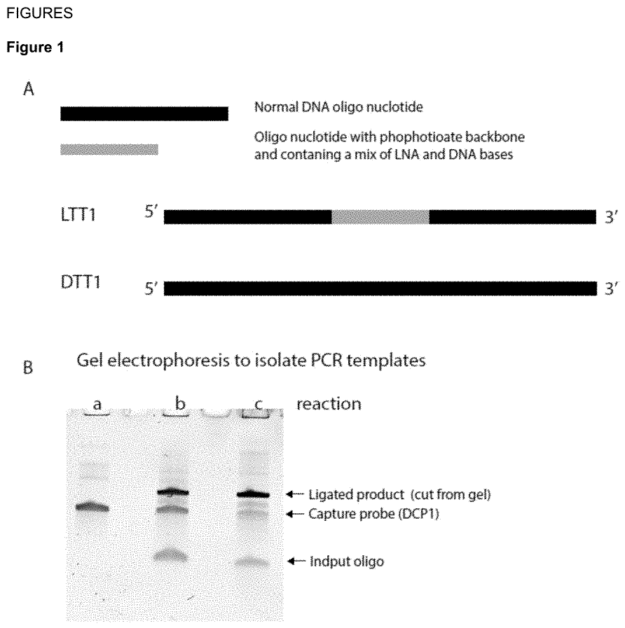

In order to be able to test various polymerase ability to read across a LNA oligo nucleotide we generated a single stranded test template molecule where a LNA oligonucleotide with a phosphorothioatebackbone (12 base pairs) is flank on both the 5′ and 3′ side of >20 bp of normal DNA bases with phosphorothioatebackbone (see FIG. 1 A), named “LNA Test Template 1” (LTT1). This template molecule was used for various later experiments. In parallel with this LNA oligo test molecule we also generated the same test template where the 12 base pairs consisted of the same sequence but where all bases where DNAs and the backbone was phosphodiester. This template is referred to as “DNA Test Template 1” (DTT1) and it served as a control oligo. These templates were used in later experiments with primers placed in the 5′ end and 3′ part of these molecules. In order for a PCR reaction to occur a polymerase must be able to extend a primer all the way across the part of the template containing the phosphodiester backbone as well as the 5′ normal DNA part. Hence this LTT1 molecules serves to test polymerases ability to copy a LNA oligo.

LTT1 and DTT1 where generated as follows:

The following oligoes where synthesized (LNA O1) or order from IDT(DNA O1).

LNA O1:
(SEQ ID NO 2)
5′-gₒcₒgₒtₒaₒaₒcₒtₒaₒgₒaₒcₒcₒaₒtₒaₒaₒgₒcₒcₒGₛAₛTₛ
AₛgₛcₛtₛtₛGₛAₛAₛ^{m}C-3′
DNA O1:
(SEQ ID NO 3)
5′-gₒcₒgₒtₒaₒaₒcₒtₒaₒgₒaₒcₒcₒaₒtₒaₒaₒgₒcₒcₒgₒaₒtₒ
aₒgₒcₒtₒtₒgₒaₒaₒc-3′

Wherein lower case letters are DNA nucleosides, uppercase letters are beta-D-oxy LNA nucleosides, ^{m} C=5 methyl cytosine beta-D-oxy LNA nucleoside, subscript o=phosphodiester internucleoside linkage, subscript s=phosphorothioate internucleoside linkages. LNA O1 is illustrated as LTT1 in FIG. 1 A, DNA O1 is illustrated as DTT1 in FIG. 1A.

These oligoes were ligated to the following DNA capture probe (DCP1)/5Phos/CGGACCAGCAAGCTTAGAGATCACGGTATCCAGATTCGCTCATAGTACACAACT GCC/iSp18/TCCGGTTCAA/3AmMO/(SEQ ID NO 4)

(All nucleosides are phosphodiester linked DNA nucleosides; “/5Phos/” indicates 5′ phosphate group; /iSp18/indicates 18-atom hexa-ethyleneglycol spacer; /3AmMO/indicates a 3′Amino modifier). Note in sequence listing, the base sequence of the probes disclosed herein is provided without the modifications specified, and in some instances RNA bases illustrated as DNA bases—In the case of discrepancy, the sequence and modifications of the sequences in the examples takes preference over the disclosure in the sequence listing.

Ligation Reaction:

The following ligation reaction was setup in PCR tubes:

a: 2 ul H2O+2 uL DCP1 (100 uM)

b: 2 ul LNA O1 (10 uM)+2 uL DCP1 (100 uM)

c: 2 ul LNA O1 (10 uM)+2 uL DCP1 (100 uM)

The mixes were heated 3 min 55 C and then cool to 4 C.

To each tube was added:

2 ul T4 DNA Ligase Buffer (Thermo Scientific)

6 ul PEG (50%)

6 ul H2O

2 ul T4 DNA Ligase (Thermo Scientific)

The mixes were vortexed and ligation was done at the following condition 3× cycles (16 C; 20 min, 25; 10 min, 37 C; 1 min) then 75 C 10 min then 4 C hold.

Gel Electrophoresis:

To each of the above mentioned reactions an equal volume of 2× Novex® TBE-Urea Sample Buffer (Thermo Fisher Scientific) has been added and samples were heat denatured for 2 min at 95° C. and placed on ice. Fifteen μl of thus prepared samples were loaded onto Novex® TBE-Urea Gels, 15%, 15 well (Thermo Fisher Scientific) and the electrophoresis was conducted for 75 min with constant voltage of 180 V. DNA was stained using SYBR Gold Nucleic Acid Gel Stain (Thermo Scientific) for 10 min. Gel was visualized with ChemiDoc Touch Imaging System (Bio Rad) on a Blue Tray (see FIG. 1 B).

The band containing the ligation product between DCP1 and the oligonucleotides were cut from the gel. The Gel piece crunch and soaked in 500 ul TE buffer over night the extract the ligated oligoes. Following the soaking the ligated oligoes were up washed and concentrated using

Amicon® Ultra-0.5 Ultracel-3 membrane, 3 kDa columns. The concentration of the two template oligoes (LTT1 and DTT1) were measured on a nanodrop and normalized to the same concentration.

### Example 2: Standard PCR Amplification on LNA Oligo Containing Template is not Possible

LTT1, DTT1 and the capture probe (DCP1) were used as template molecules in a standard emulsion PCR reaction performed with QX200™ ddPCR™ EvaGreen Supermix. Droplets were generated on a AutoDG(BioRad) using Automated Droplet Generation Oil for EvaGreen. Following PCR cycling the droplets were read on a QX200 droplet reader(BioRad).

The PCR reaction was setup using a DCP1 specific primer (DCP1_primer1: GCAGTTGTGTACTATGAGCGA, SEQ ID NO 5) and a forward primer binding to the 5′ end of the LTT1 and DTT1 (TT1_primer1: GCGTAACTAGACCATAAGCC, SEQ ID NO 6). A second reaction was also performed were additional standard Taq Polymerase (New England Biolabs) was added. This was done to see if addition of standard Taq would improve the read through of the LTT1.

22 uL PCR reaction setup:

| Normal QX200 ddPCR | QX200 ddPCR Evagreen |
|---|---|
| Evagreen | Supermix reaction with added |
| Supermix reaction | standard Taq Polymerase |
| 11 uL QX200 ddPCR Evagreen | 11 uL QX200 ddPCR Evagreen |
| Supermix (Biorad) | Supermix (Biorad) |
| 0.2 ul TT1_primer1 (10 uM) | 0.2 ul TT1_primer1 (10 uM) |
| 0.2 ul DCP1_primer1 (10 uM) | 0.2 ul DCP1_primer1 (10 uM) |
| 8.6 uL H₂O | 0.1 ul Taq Polymerase |
| 2 uL Sample (10 fM) | (NEB 5000 units/mL) |
| (LTT1, DCP1 or DTT1) | 8.5 uL H₂O |
| | 2 uL Sample (10 fM) |
| | (LTT1, DCP1 or DTT1) |

EvaGreen ddPCR program:

| | | |
|---|---|---|
| Initial Denauration: | 95° C. | 5 min |
| 40x cycles of: | | |
| Denaturation: | 95° C. | 30 sek |
| Anneling/extention : | 52.5° C. | 1 min |
| Droplet stabilization: | 4° C. | 5 min |
| | 90° C. | 5 min |
| Hold: | 4° C. | inf. |

Results:

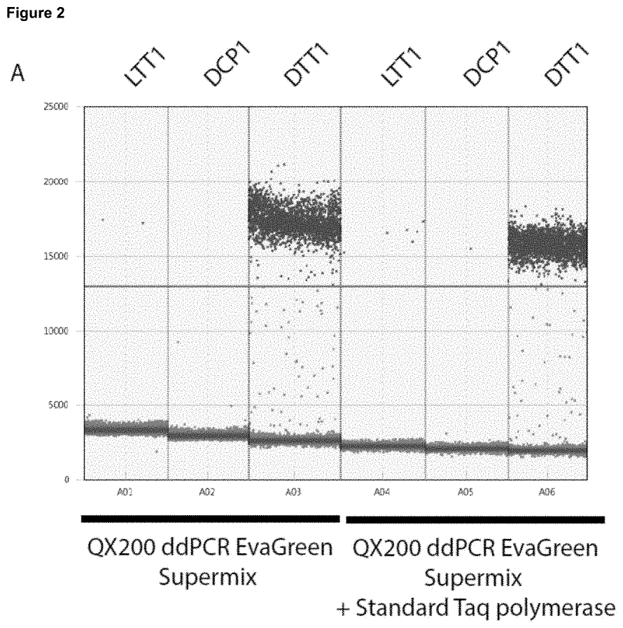

FIG. 2 Panel A shows a 1D plot of the fluoresce intensities of the droplets in the 6 different PCR reactions. It can clearly be seen that PCR amplification of the DTT1 template is feasible since many positive droplets appear. However we saw none or very few positive droplets in the PCR reaction using the LTT1 or the DCP1. The same results were seen regardless of extra addition of standard Taq Polymerase. This illustrates that Taq Polymerase is almost never able to read all the way across a LNA containing oligo with a phosphorotioate backbone.

### Example 3: Testing Various Enzymes Ability in a 1. Strand Synthesis Assay of LTT1

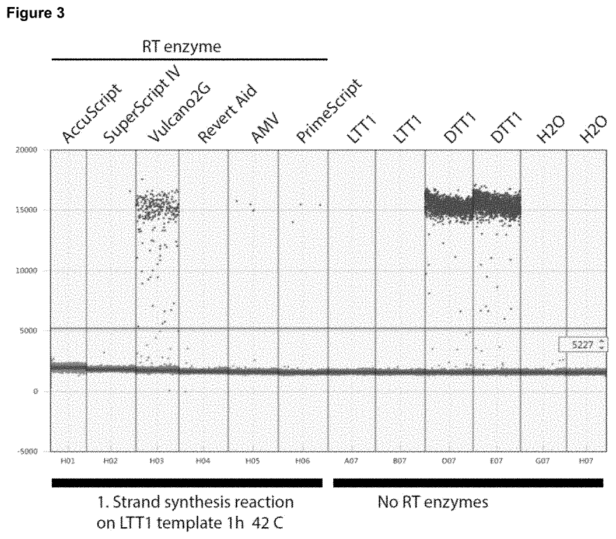

Since a Taq Polymerase is unable to read across the LTT1 template under normal conditions we tested a number of commercial available Reverse Transcriptase (RT) Polymerases, to see if RT enzymes were able to read across the LNA oligo stretch. This was done by setting up a 1.strand copy reaction were different polymerase tried to copy the LTT1 using the DCP1_primer1 as primer. Secondly; the quantity of generated intact LTT1 1.strand copy were tested in a ddPCR reaction using the DCP1_primer1 and TT1_primer1 as described in Example 2. FIG. 3 displays the results of testing the following 6 polymerases: AccuScript Hi-Fi Reverse Transcriptase from Agilent, SuperScript IV Reverse Transcriptase 200 U/ul from Thermo Scientific, Volcano2G DNA polymerase 5 U/ul from MyPols, RevertAid Reverse Transcriptase 200 U/ul from Thermo Scientific, AMV Reverse Transcriptase 10 Units/ul from New England BioLabs, PrimeScript Reverse Transcriptase (PrimeScript RT Reagent Kit) from Takara.

10 ul 1.Strand synthesis reactions:

All reaction contained (1 ul LTT1 (31 pM), 0.5 ul DCP1_primer1 (10 uM) and water add 10 ul. The different enzymes were run with the buffer provided from the vendor.
a) 1 ul Accuscript, 1 ul 0.1M DTT, 1 ul buffer, 1 ul dNTP (10 mM)
b) 0.5 ul SuperScript IV, 0.5 ul 0.1M DTT, 2 ul first strand buffer, 1 ul dNTP (10 mM)
c) 0.2 ul Vulcano2G, 2 ul Vulcano Buffer, 1 ul dNTP (10 mM)
d) 0.5 ul RevertAid, 2 ul Reaction Buffer, 1 ul dNTP (10 mM)
e) 0.5 ul AMV, 0.5 ul 0.1M DTT, 2 ul cDNA synthesis buffer, 1 ul dNTP (10 mM)
f) 0.5 ul PrimeScript, 2 ul PrimeScript Buffer, 1 ul dNTP (10 mM)

All components were added except the enzyme at the mixes were headed to 65 C 5 min then on ice 1 min before the RT enzyme was finally added. 1.Strand synthesis was done 1 hour at was done at the following temperatures for each condition (55 C, 54.2 C, 52.5 C, 50 C, 47.1 C, 44.6 C, 42.9 C, 42 C). Then 80 C 10 min and cool and hold at 4 C.

All 1.strand reactions were diluted 200× in water, and 2 ul sample was used as input for a normal QX200™ ddPCR™ EvaGreen Supermix PCR reaction as described in Example 2. 2 ul LTT1(15.5 fM), DTT1(15.5 fM) or H2O was included as negative and positive controls. 2 ul input of 15.5 fM is equivalent to the number of LTT1 molecules added from the 1.Strand Synthesis step.

Results:

FIG. 3 displays the fluoresce intensities of the droplets in the different PCR reactions. Only the results from the 42 C RT reactions are displayed in the figure. The results for the other temperatures were the same for each condition except the very small activity seen with AMV was lost above 52.5 C. We see that in general RT enzymes so no ability or very very low efficiency in reading across the LTT1 template. However we find that the Vulcano2G seems to have a considerable efficiency in reading the template. Quantification of the number of droplet compared to the ddPCR on the DTT1 template with equal number of input molecules, showed that the reading efficiency of the Vulcano2G enzyme was around 10%, meaning that one out of 10 LLT1 molecules are transcribed all the way across by the Vulcano2G enzyme. We saw around 0.1% efficiency on the AMV and Prime Script enzymes.

### Example 4: Testing PCR Additives to Allow LNA Oligo Readthrouqh by DNA Polymerase

To try to overcome the difficulties in transcribing across a LNA oligo with a polymerase we set out to test if additives in the PCR reaction could help the polymerases in reading across the LNA oligo in the LTT1. We tested 4 know PCR additives to see if they would have a beneficial effect in reading LNA oligoes, namely Tetramethylammonium (TMA) chloride, Polyethylen Glycol (PEG), Ammonium Chlorid and 1,2-propandiol. The additives were tested in a emulsion PCR reaction using the AccuStart II PCR ToughMix (QuantiBio) which contain a modified TaqPolymerase. Droplets were generated on a AutoDG(BioRad) using Automated Droplet Generation Oil for EvaGreen. Following PCR cycling the droplets were read on a QX200 droplet reader(BioRad). Separate EvaGreen dye (Biotium cat no. 31000) was added to the PCR reaction.

The following addites were tested for the ddPCR reaction: TMA (1 mM, 5 mM, 10 mM, 20 mM, 40 mM, 60 mM, 80 mM, 100 mM), PEG(0%, 0.1%, 0.5%, 1%, 2%, 3%, 4%, 5%), Ammonium Chloride (1 mM, 5 mM, 10 mM, 20 mM, 40 mM, 60 mM, 80 mM, 100 mM), 1,2-propandiol (0.2M, 0.4M, 0.6M, 0.8M, 1M, 1.2M, 1.6M, 2M).

PCR reaction mix (22 ul):

11 ul AccuStart II PCR ToughMix

0.2 ul TT1_primer1 (10 uM)

0.2 ul DCP1_primer1 (10 uM)

0.5 ul EvaGreen dye (40×)

X uL PCR additive

2 uL LTT1 (100 fM)

H2O ad 22 uL

ddPCR program:

| | | |
|---|---|---|
| Initial Denauration: | 95° C. | 5 min |
| 40x cycles of: | | |
| Denaturation: | 95° C. | 30 sek |
| Anneling/extention : | 52.5° C. | 1 min |
| Droplet stabilization: | 4° C. | 5 min |
| | 90° C. | 5 min |
| Hold: | 4° C. | inf. |

Results:

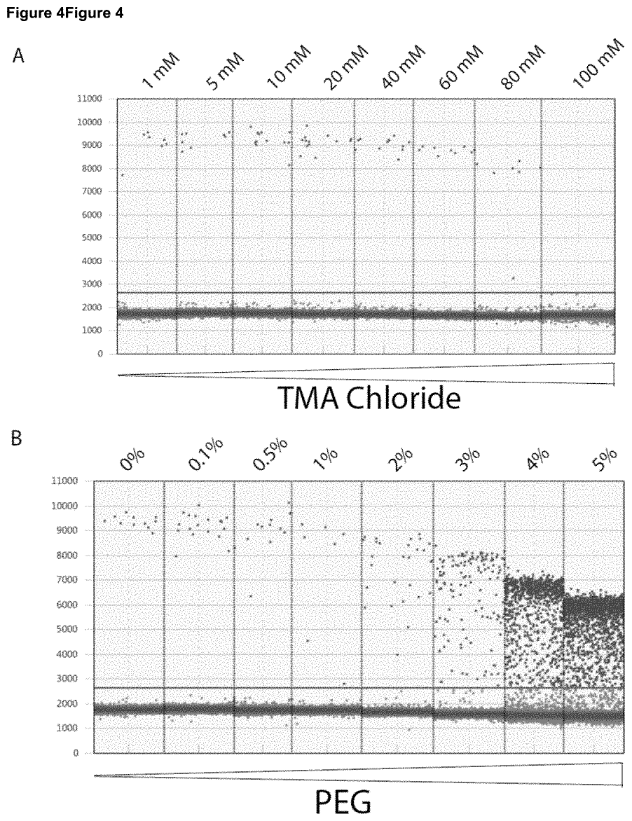

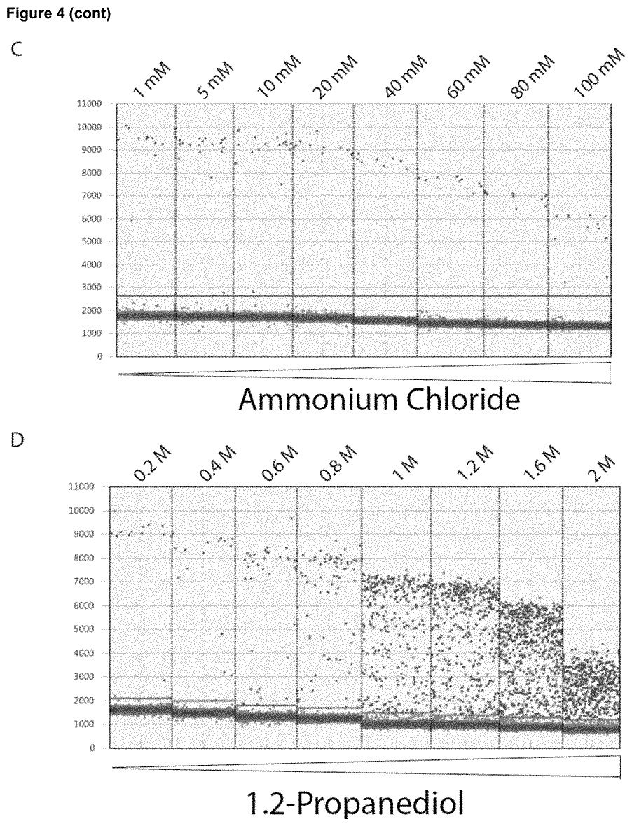

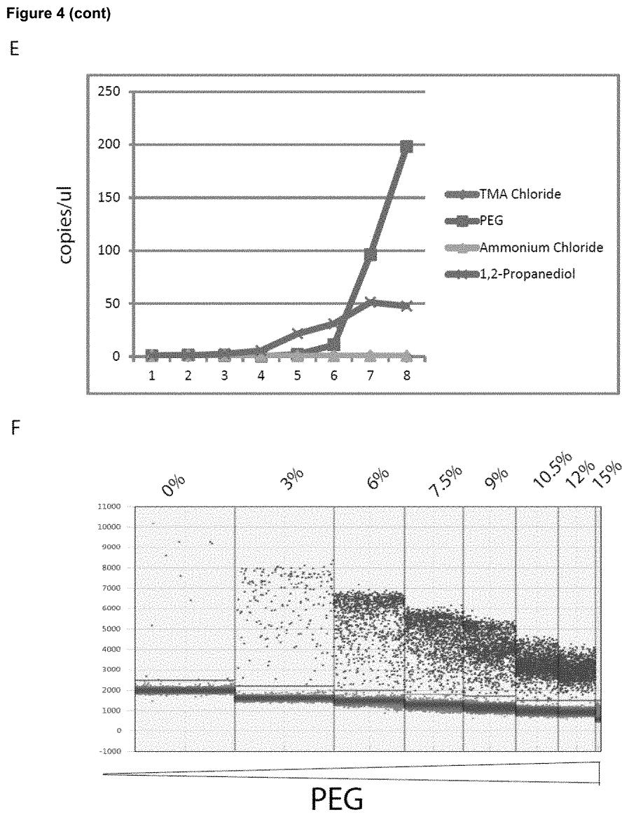

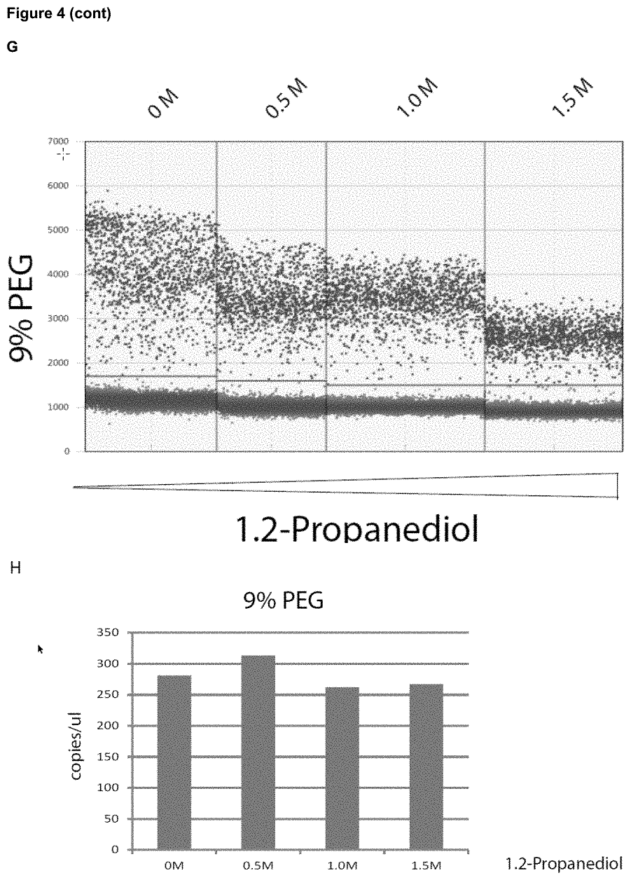

FIG. 4 displays the results of the ddPCR with additives. The results are displayed as 1D plots showing the Florence intensities of all the droplets. The results showed that addition of TMA Chloride and Ammonium Chloride didn't result in any improvement of the LNA read through (FIG. 4 panel A and C). However we see that increasing concentrations of PEG results in an increase in the amount of positive droplets. The positive effect start at 3% PEG and increased for both 4% and 5% (FIG. 4 panel B). We also observed a positive effect with the addition of 1,2-Prooanediol were we saw an increase in the amounts of positive droplets starting around 0.8M 1,2-Propanediol (FIG. 4 panel D). Although increasing the 1.2-Propanediol concentration above 1.2M did resulted in a bit more positive droplets, it also resulted in a decrease in the fluoresce intensity of the positive droplets. Taken together we conclude that addition of PEG or 1,2-propanediol enables a Taq polymerase to increase its ability to read across a LNA oligo with phosphorotioate backbone dramatically. FIG. 4 Panel E displays the concentration of LTT1 molecules detected showing that PEG clearly increase LTT1 detection and that PEG is a better additive compared to 1.2-Propanediol that also gave some increased ability to read the LTT1 template.

Since we didn't see the saturation of the positive effect of PEG we performed a second experiment with the same setup but where we increased the concentration of PEG in the reaction further. (FIG. 4 Panel F). When the concentration of PEG is increased above 9% we start to see that the droplets start to collapse although the numbers of positive droplet are the same. When the concentration of PEG was 15% we saw a complete collapse of the droplets. Finally we tested if the combination of PEG and 1,2-propanediol increased the oligo read-through further. FIG. 4 panel G displays the ddPCR reaction with 9% PEG and 0M, 0.5M, 1.0M or 1.5M 1.2-propanediol accordingly, showing that no clear benefit was see by co-adding the 1.2-Propanediol to further enhance the reaction. In general we didn't see a benefit of adding additional 1.2-Propanediol to the ddPCR reaction when the amount of PEG was above 6% (data not shown).

### Example 5: PEG and/or 1.2-Propandiol Enables Some DNA Polymerases to Read LNA Oligoes

In example 4 we showed that addition of PEG and 1.2-Propanediol was beneficial for the successful amplification of a LTT1 template molecule when we used the Accustart II PCR though mix (QuantaBio) that contains an undisclosed modified Taq Polymerase. To see if the PCR additives also enabled LNA oligo “read-through” for other polymerase, we tested a normal Taq Polymerase and a High fidelity Polymerase (Phusion polymerase, Thermo Scientific) by performed multiple rounds of 1. Strand synthesis of the LTT1 template. The number of intact 1.strand copy's generated was detected by Evagreen ddPCR detection as perform in experiment 2,3,4. The following 20 ul reactions were setup and performed with 0, 1, 3, 5 and 10 rounds 1.strand amplification.
a) 0.1 ul Taq Polymerase, 2 ul Taq buffer, 0.4 ul DCP1_primer1 (10 uM), 0.4 ul LTT1 (10 pM)
b) 0.1 ul Taq Polymerase, 2 ul Taq buffer, 0.4 ul DCP1_primer1 (10 uM), 0.4 ul LTT1 (10 pM), 0.5 ul 1.2-propandiol, 4 ul PEG (50%)
c) 2 ul Taq buffer, 0.4 ul DCP1_primer1 (10 uM), 0.4 ul LTT1 (10 pM), 0.5 ul 1.2-propandiol, 4 ul PEG (50%)
d) 0.2 ul Phusion Polymerase, 5×HF buffer, 0.4 ul DCP1_primer1 (10 uM), 0.4 ul LTT1 (10 pM)
e) 0.2 ul Phusion Polymerase, 5×HF buffer, 0.4 ul DCP1_primer1 (10 uM), 0.4 ul LTT1 (10 pM), 0.5 ul 1.2-propandiol, 4 ul PEG (50%)
f) 0.2 ul Phusion Polymerase, 5×GF buffer, 0.4 ul DCP1_primer1 (10 uM), 0.4 ul LTT1 (10 pM)
g) 0.2 ul Phusion Polymerase, 5×GF buffer, 0.4 ul DCP1_primer1 (10 uM), 0.4 ul LTT1 (10 pM), 0.5 ul 1.2-propandiol, 4 ul PEG (50%)

1.Strand reaktion. (95° C. 5 min; X cycles of 52.5 3 min, 95° C. 30 sek; then on ice)

The 1.strand synthesis reaction was diluted 50× and 2 ul was used as input in a Evagreen ddPCR reaction as described in example 2.

Results:

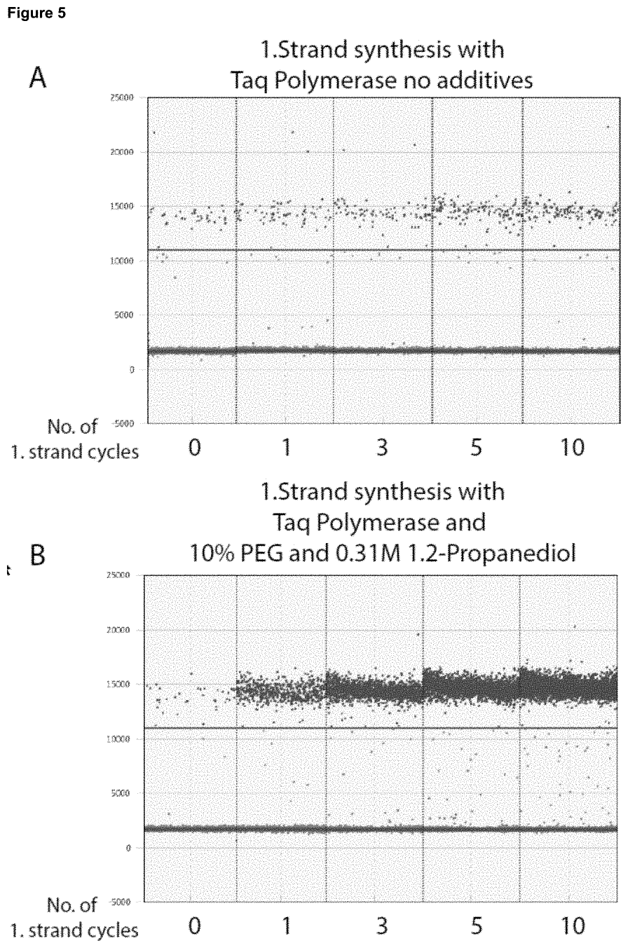

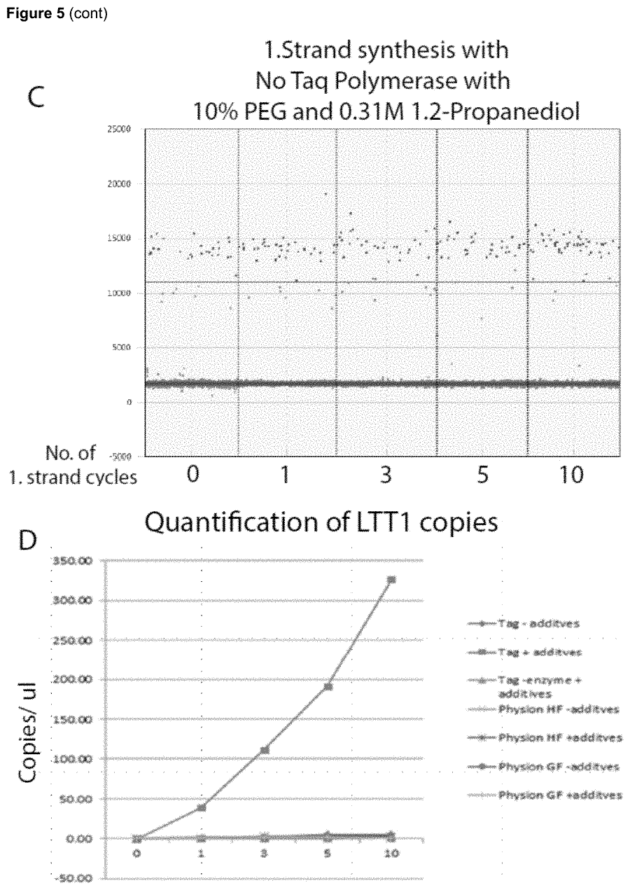

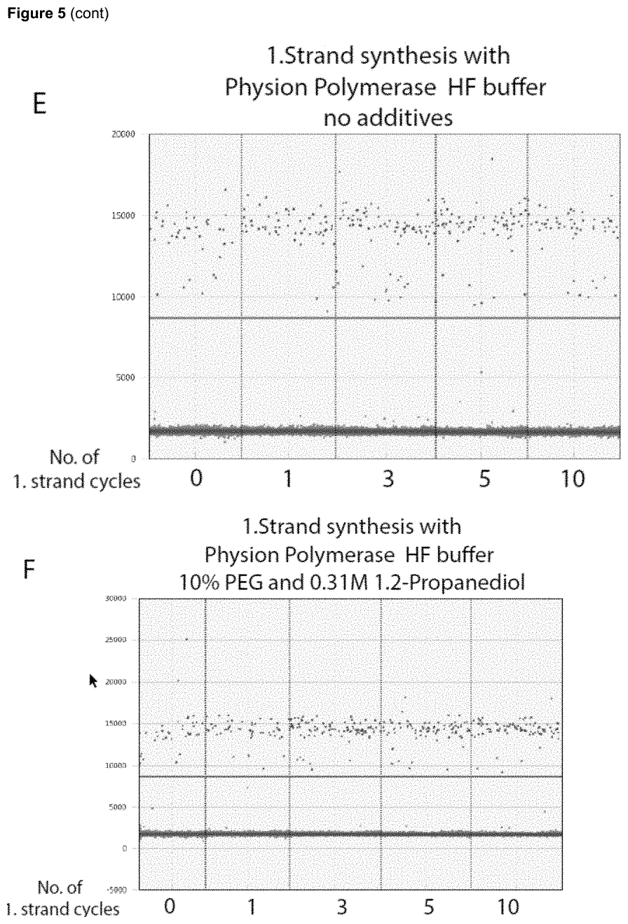

FIG. 5 displays the results of the ddPCR reaction on the 1.strand synthesis. FIG. 5 panel A displays the ddPCR reaction on 1.strand Taq polymerase synthesis without PCR additives. As can be seen there is hardly any increase in the number of positive droplets as a result of the 1.strand synthesis cycling, displaying again that Taq Polymerase under normal conditions cannot read across a oligo containing phosphorotioate backbone and LNA bases. FIG. 5 panel C show the same reaction but without Taq Polymerase presence. The numbers of positive droplets in 1.strand reaction without Taq polymerse are almost the same as for the reaction without additives. This showed that most of the positive droplets came from a copying of the LNA template that occurred during the Evagreen ddPCR reaction and not during the 1.strandsynthesis. FIG. 5 panel B displays the results of the ddPCR reaction when 10% PEG and 0.31M was present during the 1. Strand synthesis reaction. As can be seen the number of positives droplets are increased with the number of 1. Strand synthesis cycles demonstrating that the additives enable the standard Taq polymerase to read across the LNA oligo part of LTT1. FIG. 5 panel E and F displays the ddPCR on the 1.strand synthesis reaction with phusion DNA polymerase in HF buffer with and without the 10% PEG and 0.31M 1.3-Propanediol additives. This illustrates that Phusion polymerase cannot read across LNA oligonucleotide part of LTT1 regardless of the addition of PEG and 1.2-Propanediol and the use of buffer HF or GF buffer. FIG. 5 panel D displays a quantification of the number of detected LTT1 copies for the 7 tested conditions. As can be seen only in the reaction with Taq Polymerase and additives do we see an effective 1.Strand copying of the LTT1 template molecule. We conclude that the presence of these tested PCR additives (PEG and 1.2-propane diol) can enable some DNA polymerases, to read across an LNA oligo.

### Example 6: NGS Sequencing of LNA Oligonucleotides

In order to illustrate that sequencing of LNA oligonucleotides with full phosphorothioate backbone is possible we used the following method to sequence a mixture of 5 LNA oligonucleotides. The sequencing was performed using both a normal Taq DNA polymerase and the Vulcano2G polymerase (myPOLS) with and without the addition of PEG during 1.

Strand Synthesis.

The following LNA oligoes were mixed in a 1:1 ratio and diluted to a final conc of 1 uM each:

LNA Mix:

| Oligo | | SEQ ID |
|---|---|---|
| No | Compound | NO |
| Oligo | CTaccTgagTggcATcCT | SEQ ID |
| 1: | | NO 7 |
| Oligo | CTaccTgagTggcATcCT | SEQ ID |
| 2: | | NO 8 |
| Oligo | CAgaAaaTaCCtaCctGA | SEQ ID |
| 3: | | NO 9 |
| Oligo | GcttttaaccagagtGGC | SEQ ID |
| 4: | | NO 10 |
| Oligo | gcgtaactagaccataagccGATAgcttGAAC | SEQ ID |
| 5: | | NO 11 |

Capture Probes:

| | | SEQ ID |
|---|---|---|
| Capture probe 1 | /5Phos/CCGCAATTGGCGTGATNNNNAGATCGGAAGAGCGTCGTGT | 12 |
| index 1: | AGTCCGCATGTCGCGTGATAGGGATATCTTGCGGNNNNN/3AmMO/ | |
| Capture probe 1 | /5Phos/CCGCAATTGGACATCGNNNNAGATCGGAAGAGCGTCGTGT | 13 |
| index 2: | AGTCCGCATGTCGCGTGATAGGGATATCTTGCGGNNNNN/3AmMO/ | |
| Capture probe 1 | /5Phos/CCGCAATTGGCACTGTNNNNAGATCGGAAGAGCGTCGTGT | 14 |
| index 3: | AGTCCGCATGTCGCGTGATAGGGATATCTTGCGGNNNNN/3AmMO/ | |
| Capture probe 1 | /5Phos/CCGCAATTGGATTGGCNNNNAGATCGGAAGAGCGTCGTGT | 15 |
| index 4: | AGTCCGCATGTCGCGTGATAGGGATATCTTGCGGNNNNN/3AmMO/ | |

Capture probe RT primer:
(SEQ ID NO 16)
CTATCACGCGACATGCGG

1. Ligation reaction:

2 ul LNA mix was mixed with 2 ul Capture probe index 1 (10 uM)

2 ul LNA mix was mixed with 2 ul Capture probe index 2 (10 uM)

2 ul LNA mix was mixed with 2 ul Capture probe index 3 (10 uM)

2 ul LNA mix was mixed with 2 ul Capture probe index 4 (10 uM)

Then mix was heated to 55 C then cool to 4 C.

16 ul Ligation mix was added to each tube:

Per 20 ul reaction: (2 ul T4 Ligation buffer, 6 ul PEG, 6 ul H2O, 2 ul T4 Ligase)

Gel Electrophoresis:

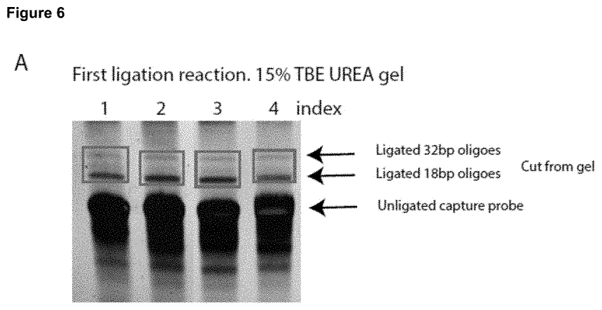

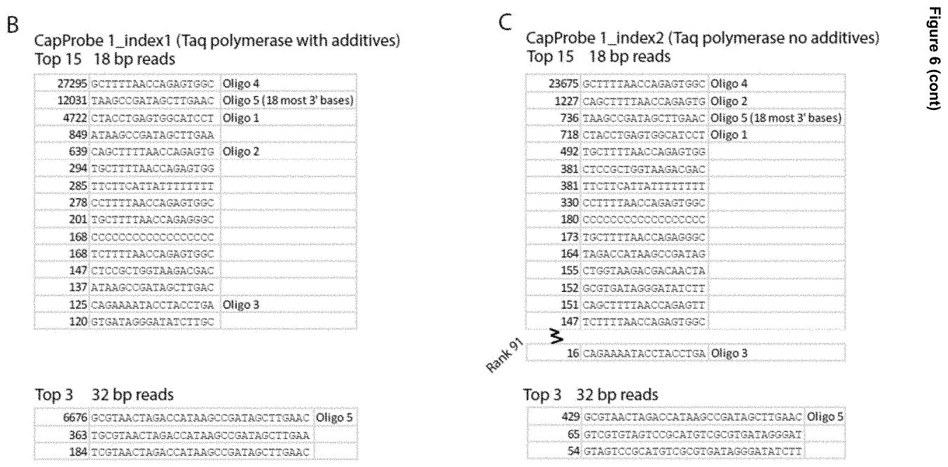

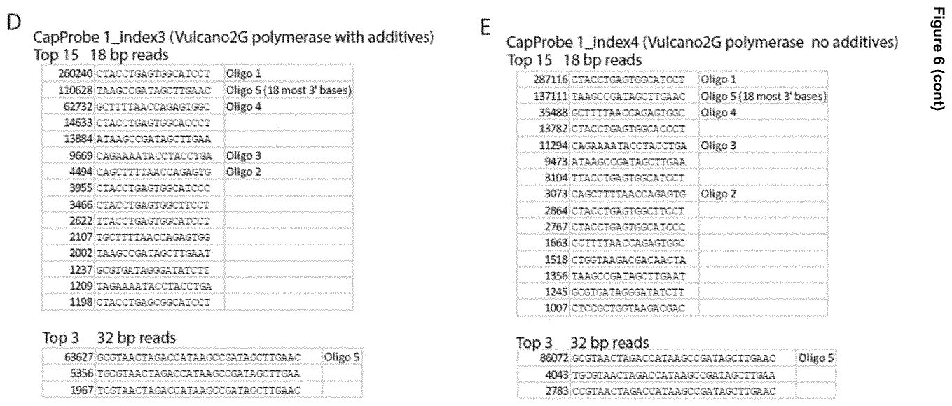

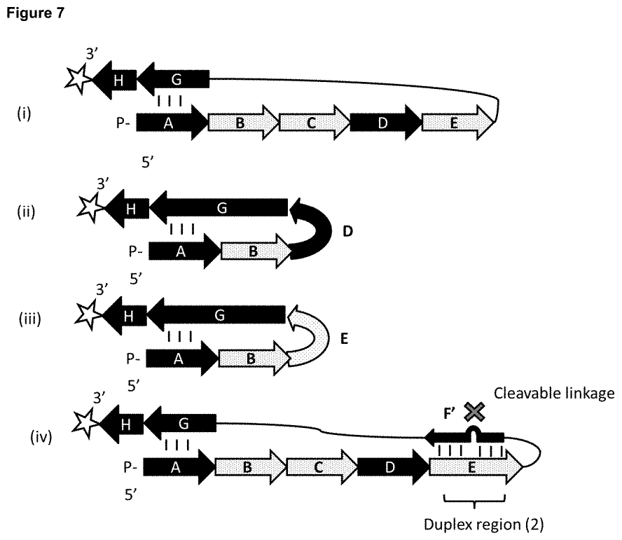

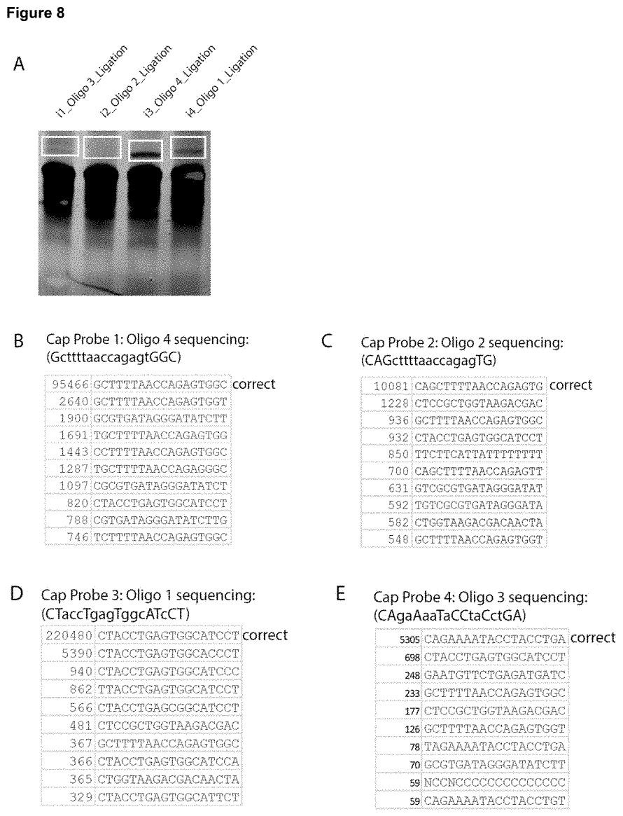

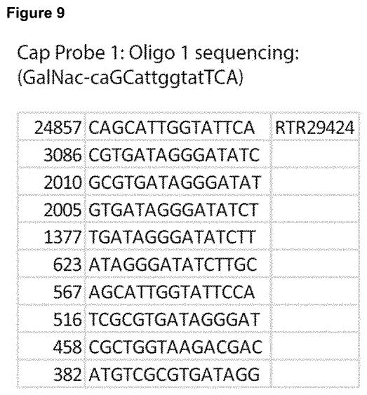

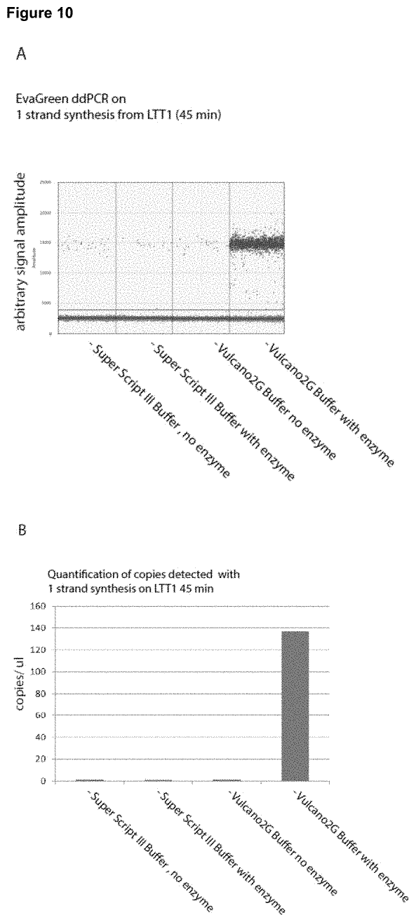

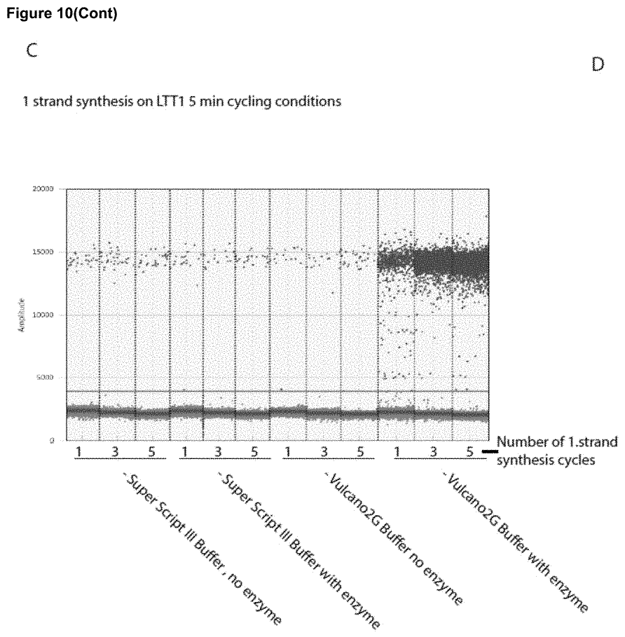

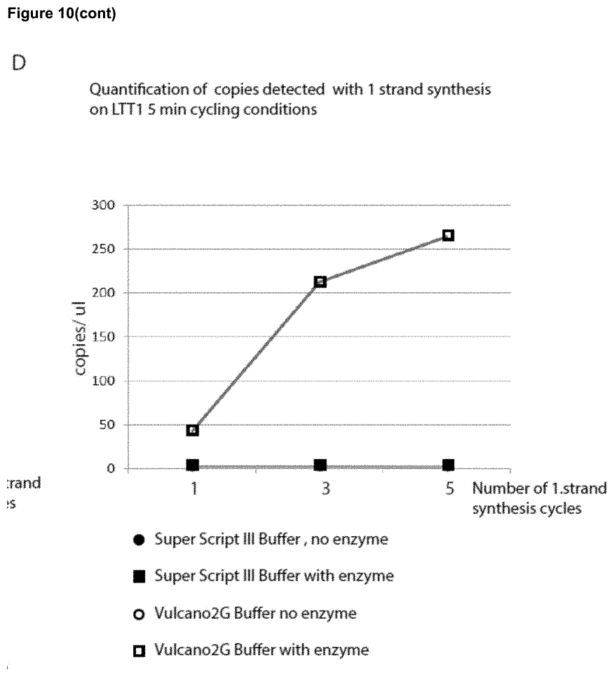

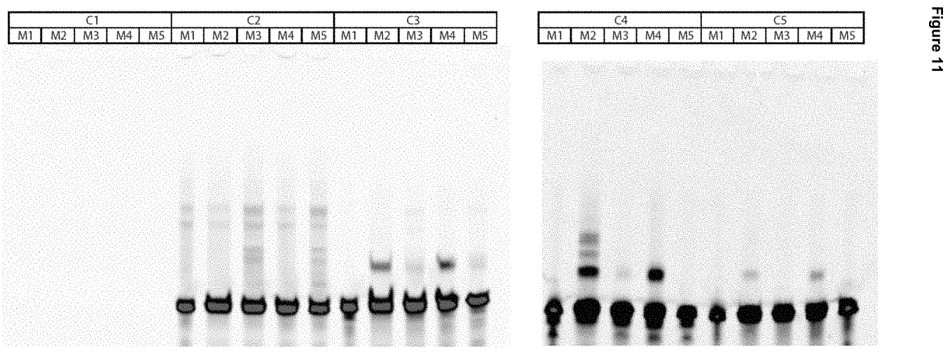

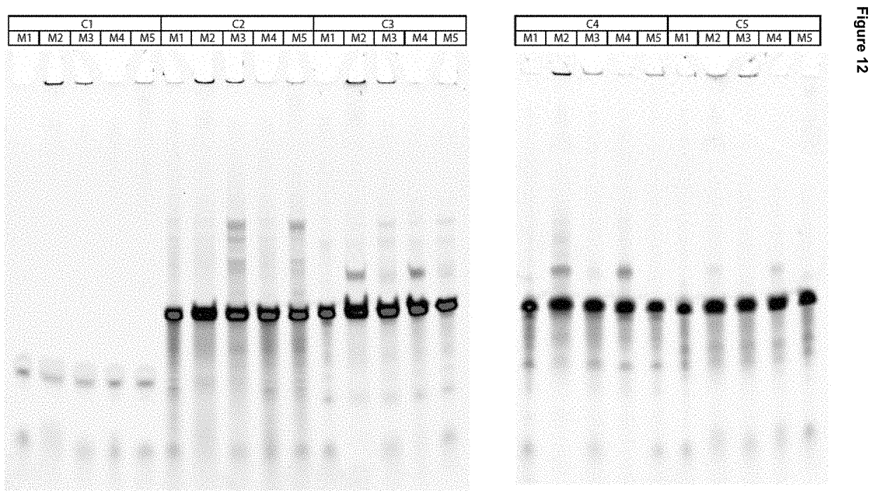

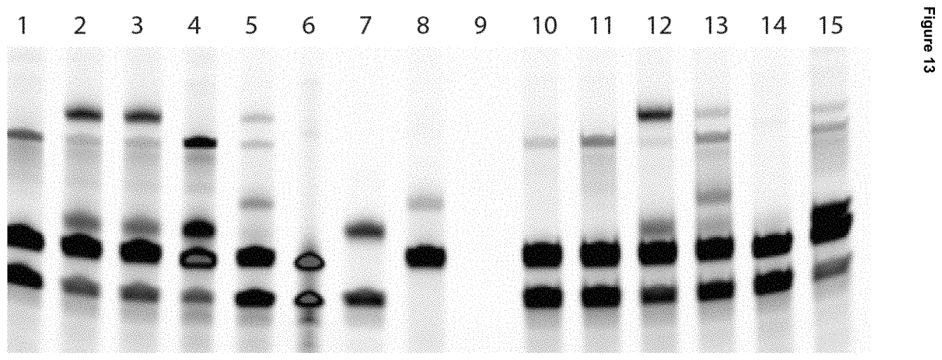

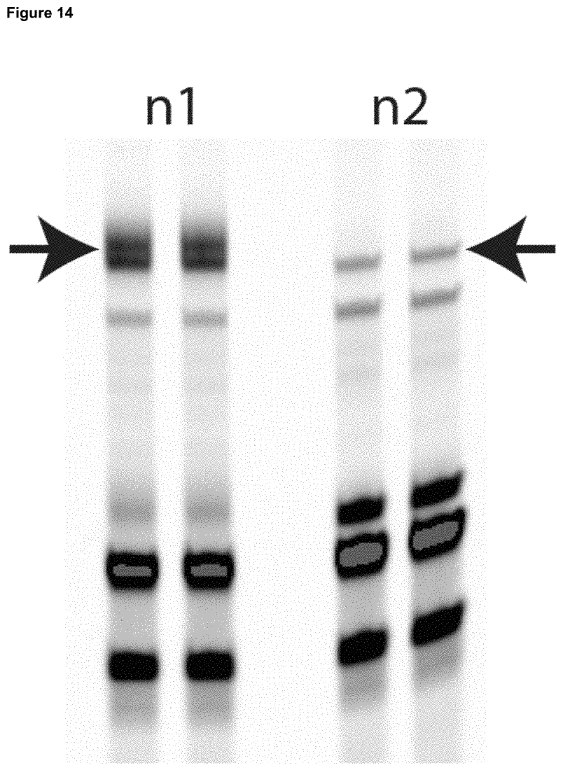

To each of the above mentioned reactions an equal volume of 2× Novex® TBE-Urea Sample Buffer (Thermo Fisher Scientific) were added and samples were heat denatured for 2 min at 95° C. and placed on ice. Fifteen μl of thus prepared samples were loaded onto Novex® TBE-Urea Gels, 15%, 15 well (Thermo Fisher Scientific) and the electrophoresis was conducted for 75 min with constant voltage of 180 V. DNA was stained using SYBR Gold Nucleic Acid Gel Stain (Thermo Scientific) for 10 min. Gel was visualized with ChemiDoc Touch Imaging System(Bio Rad) on a Blue Tray (see FIG. 6 A).

The band containing the ligation product between the capture probes and the oligonucleotides were cut from the gel. The cut area is indicated by a red box in FIG. 6A. The Gel pieces were crunch and soaked in 500 ul TE buffer over night the extract the ligated oligoes. Following the soaking the ligated oligoes were washed and concentrated using Amicon Ultra 0.5 mL centrifugal MWCO 3 kDa filters. Finally the samples were concentrated to approximately 10 ul using a speedvac.

1.Strand Synthesis and Purification:

4 different protocols were used to produce 1.strand copys of the ligated LNA oligoes.

| Capture probe index | Capture probe index | Capture probe index | Capture probe index |
|---|---|---|---|
| 1 reaction | 2 reaction | 3 reaction | 4 reaction |
| 2 ul: 10x standard | 2 ul: 10x standard | 4 ul: 5x Vulcano2G | 4 ul: 5x Vulcano2G |
| Taq buffer | Taq buffer | buffer | buffer |
| 0.2 ul: Taq DNA | 0.2 ul: Taq DNA | 0.4 ul: Vulcano2G | 0.4 ul: Vulcano2G |
| Polymerase | Polymerase | Polymerase | Polymerase |
| 0.5 ul: 1,2-Propandiol | | 0.5 ul: 1,2-Propandiol | |
| 4 ul: PEG (50%) | | 4 ul: PEG (50%) | |
| 0.5 ul: dNTP (10 uM) | 0.5 ul: dNTP (10 uM) | 0.5 ul: dNTP (10 uM) | 0.5 ul: dNTP (10 uM) |
| 0.4 ul: Capture RT | 0.4 ul: Capture RT | 0.4 ul: Capture RT | 0.4 ul: Capture RT |
| Primer (10 uM) | Primer (10 uM) | Primer (10 uM) | Primer (10 uM) |
| 4 ul: Ligation | 4 ul: Ligation | 4 ul: Ligation | 4 ul: Ligation |
| template | template | template | template |
| H20 ad 20 ul | H20 ad 20 ul | H20 ad 20 ul | H20 ad 20 ul |

1. Strand synthesis was performed on a thermocycler with the following program: 95 C; 3 min 10×(55 C; 5 min, 72 C; 1 min) then hold 4 C

The 1.strandsynthesis reaction were purified using the Monarch® PCR & DNA Cleanup Kit (New England Biolabs) using the manufactures Oligonucleotide Cleanup Protocol. Samples were eluted in 10 ul Elution buffer.

2.Ligation Reaction:

Capture Probe 2
(SEQ ID NO 17)
/5Phos/CCGCAAGATCGGAAGAGCGGTTCAGCAGGAATGCATATGCTTG
CGGNNNNNN/3AmMO/

8 ul 1.strand synthesis reaction was mix with 2 ul Capture probe 2 (1 uM)

Then mix was heated to 55 C then cool to 4 C.

16 ul Ligation mix was added to each tube:

Per 20 ul reaction: (2 ul T4 Ligation buffer, 6 ul PEG, 6 ul H2O, 2 ul T4 Ligase). Ligation:

2×(40; 2 min, 160; 2 hours, 22 C 5 min) 75 C; 10 min then hold on 4 C.

PCR Amplification of NGS Library:

NGS_PCR_primer1
(SEQ ID NO 18)
AATGATACGGCGACCACCGAGATCTACACTCTTTCCCTACACGACGCTCT
TCCGATCT
NGS_PCR_primer2
(SEQ ID NO 19)
CAAGCAGAAGACGGCATACGAGATCGGTCTCGGCATTCCTGCTGAACCGC
TCTTCCGATCTT

PCR amplification of the ligated 1.strand synthesis was performed with a phusion DNA polymease using the NGS_PCR_primer1 and 2. These primers contain 5′ overhang compatible with illuminas TruSeq NGS protocol.

| | |
|---|---|
| 4 ul: | 5× HF Buffer |
| 0.4 ul: | dNTP (10 uM) |
| 1 ul: | NGS_PCR_primer1 (10 uM) |
| 1 ul: | NGS_PCR_primer2 (10 uM) |
| 0.2 ul: | Phusion DNA polymerase |
| 11.4 ul: | H₂O |
| 2 ul: | Sample from 2. Ligation reaction |

PCR cycling: 98 C; 30s, 15×(98 C; 15 s, 60 C; 20s, 72 C; 20s), 72 5 min then hold 4 C The PCR product was purified on a QIAquick PCR purification kit (Qiagen) according to manufactures instructions and eluted in 30 ul H2O.

NGS Setup:

The 4 reactions were normalized to the same concentration and were pooled together to create a 10 nM NGS library. A phiX control mix was spiked into this sample to a final concentration of 20% of the total molecules to give sequence variation for the subsequent illumine sequencing. The NGS library was prepared according to Illumina's Denature and Dilute Library Guide for the MiniSeq System. The library was sequenced on an Illumina miniSeq system using a MID output cassette. The sequencing was setup to generate fastq files use only read 1 and without indexes performing 151 cycles.

NGS Data Analysis:

The generated fastq files were imported into the CLC Genomics Workbench 10 software (Qiagen). The reads was separated according to the barcode build into the different Capture Probes 1 and the remaining reading from capture probe 1 was trimmed away from the 5′end of the reads. Subsequently the sequence originating from the Capture Probe 2 was trimmed away from the 3′end leaving behind only the sequence inserted between the capture probes 1 and 2. Using awk command lines all reads shorter than 18 was then trimmed away, and finally al reads longer than 18 bp or 32 bp was trimmed down to 18 or 32 bp by removing bases from the 3 end of the sequencing read. The number of unique reads was quantified and the top 10 most frequent reads are presented in FIG. 6 panel B. In FIG. 6 the reads have been reversed complemented in order to reads the original LNA oligo in a 5′->3′ sense manner.

### Example 7

### Example 1: NGS Sequencing of LNA as Quality Control(QC)

We illustrate here that sequencing of fully phosphorothioated LNA oligoes for QC proposes is possible.

The following LNA oligoes were used for sequencing.

LNA Mix:

| (SEQ ID NO 7) | |
|---|---|
| Oligo 1: | CTaccTgagTggcATcCT |
| (SEQ ID NO 21) | |
| Oligo 2: | CAGcttttaaccagagTG |
| (SEQ ID NO 9) | |
| Oligo 3: | CAgaAaaTaCCtaCctGA |
| (SEQ ID NO 10) | |
| Oligo 4: | GcttttaaccagagtGGC |

Capture Probes:

Capture probe 1 index 1:
(SEQ ID NO 12)
/5Phos/CCGCAATTGGCGTGATNNNNAGATCGGAAGAGCGTCGTGTAGT
CCGCATGTCGCGTGATAGGGATATCTTGCGGNNNNN/3AmMO/
Capture probe 1 index 2:
(SEQ ID NO 13)
/5Phos/CCGCAATTGGACATCGNNNNAGATCGGAAGAGCGTCGTGTAGT
CCGCATGTCGCGTGATAGGGATATCTTGCGGNNNNN/3AmMO/
Capture probe 1 index 3:
(SEQ ID NO 14)
/5Phos/CCGCAATTGGGCCTAANNNNAGATCGGAAGAGCGTCGTGTAGT
CCGCATGTCGCGTGATAGGGATATCTTGCGGNNNNN/3AmMO/
Capture probe 1 index 4:
(SEQ ID NO 15)
/5Phos/CCGCAATTGGTGGTCANNNNAGATCGGAAGAGCGTCGTGTAGT
CCGCATGTCGCGTGATAGGGATATCTTGCGGNNNNN/3AmMO/

Capture probe 1 RT primer:
(SEQ ID NO 16)
CTATCACGCGACATGCGG

First Ligation reaction:

2 ul Oligo 1 was mixed with 2 ul Capture probe 1 index 3 (10 uM)

2 ul Oligo 2 was mixed with 2 ul Capture probe 1 index 2 (10 uM)

2 ul Oligo 3 was mixed with 2 ul Capture probe 1 index 4 (10 uM)

2 ul Oligo 4 was mixed with 2 ul Capture probe 1 index 1 (10 uM)

Then mix was heated to 55 C then cool to 4 C.

16 ul Ligation mix was added to each tube:

Per 20 ul reaction: (2 ul T4 Ligation buffer, 6 ul PEG, 6 ul H2O, 2 ul T4 Ligase). Ligation:

2×(4 C; 2 min, 16 C; 20 min, 22 C 5 min, 30 C 1 min) 75 C; 10 min then hold on 4 C.
Gel electrophoresis:

To each of the above mentioned reactions an equal volume of 2× Novex® TBE-Urea Sample Buffer (Thermo Fisher Scientific) were added and samples were heat denatured for 2 min at 95° C. and placed on ice. Fifteen μl of the prepared samples were loaded onto Novex® TBE-Urea Gels, 15%, 15 well (Thermo Fisher Scientific) and the electrophoresis was conducted for 75 min with constant voltage of 180 V. DNA was stained using SYBR Gold Nucleic Acid Gel Stain (Thermo Scientific) for 10 min. Gel was visualized with ChemiDoc Touch Imaging System(Bio Rad) on a Blue Tray (see FIG. 8 A).

The band containing the ligation product between the capture probe and the oligo were cut from the gel. The cut area is indicated by a white box in FIG. 1A. The Gel pieces were crunch and soaked in 500 ul TE buffer over night the extract the ligated oligoes. Following the soaking the ligated oligoes were washed and concentrated using Amicon Ultra 0.5 mL centrifugal MWCO 3 kDa filters. Finally the samples were concentrated to approximately 10 ul using a speedvac.
1.Strand synthesis and purification:

1. Strand synthesis was performed using the Vulcano2G DNA Polymerase in a 20 ul reaction:

4 ul: 5× Vulcano2G buffer

0.4 ul: Vulcano2G Polymerase

0.5 ul: dNTP (10 uM)

0.4 ul: Capture RT Primer (10 uM)

4 ul: Ligation template

H20 ad 20 ul

Using the following program on a thermocycler:

95 C; 3 min 10×(55 C; 5 min, 72 C; 1 min) then hold 4 C

The 1.strandsynthesis reaction were purified using the Monarch® PCR & DNA Cleanup Kit (New England Biolabs) using the manufactures Oligonucleotide Cleanup Protocol. Samples were eluted in 10 ul Elution buffer.
2.Ligation reaction:

Capture Probe 2
(SEQ ID NO 17)
/5Phos/CCGCAAGATCGGAAGAGCGGTTCAGCAGGAATGCATATGCTTG
CGGNNNNNN/3AmMO/

8 ul 1.strand synthesis reaction was mix with 2 ul Capture probe 2 (1 uM)

Then mix was heated to 55 C then cool to 4 C.

16 ul Ligation mix was added to each tube:

Per 20 ul reaction: (2 ul T4 Ligation buffer, 6 ul PEG, 6 ul H2O, 2 ul T4 Ligase). Ligation:

2×(4 C; 2 min, 16 C; 2 hours, 22 C 5 min) 75 C; 10 min then hold on 4 C.
PCR amplification of NGS Library:

NGS_PCR_primer1
AATGATACGGCGACCACCGAGATCTACACTCTTTCCCTACACGACGCTCT
TCCGATCT
NGS_PCR_primer2
CAAGCAGAAGACGGCATACGAGATCGGTCTCGGCATTCCTGCTGAACCGC
TCTTCCGATCTT

PCR amplification of the ligated 1.strand synthesis was performed with a phusion DNA polymerase using the NGS_PCR_primer1 and 2. These primers contain 5′ overhang compatible with illuminas TruSeq NGS protocol.

| | |
|---|---|
| 4 ul: | 5x HF Buffer |
| 0.4 ul: | dNTP (10 uM) |
| 1 ul: | NGS_PCR_primer1 (10 uM) |
| 1 ul: | NGS_PCR_primer2 (10 uM) |
| 0.2 ul: | Phusion DNA polymerase |
| 11.4 ul: | H₂O |
| 2 ul: | Sample from 2. Ligation reaction |

PCR cycling: 98 C; 30s, 15×(98 C; 15 s, 60 C; 20s, 72 C; 20s), 72 5 min then hold 4 C

The PCR product was purified on a QIAquick PCR purification kit (Qiagen) according to manufactures instructions and eluted in 30 ul H₂O.
NGS setup:

The 4 reactions were normalized to the same concentration and were pooled together to create a 10 nM NGS library. A phiX control mix was spiked into this sample to a final concentration of 20% of the total molecules to give sequence variation for the subsequent illumine sequencing. The NGS library was prepared according to Illuminas Denature and Dilute Library Guide for the MiniSeq System. The library was sequenced on an Illumina miniSeq system using a MID output cassette. The sequencing was setup to generate fastq files use only read 1 and without indexes performing 151 cycles.
NGS data analysis:

The generated fastq files were imported into the CLC Genomics Workbench 10 software (Qiagen). The reads was separated according to the barcode build into the four Capture Probes 1's and the remaining reading from capture probe 1 was trimmed away from the 5′end of the reads. Subsequently the sequence originating from the Capture Probe 2 was trimmed away from the 3′end leaving behind only the sequence inserted between the capture probes 1 and 2. Using awk command lines all reads shorter than 18 was then trimmed away, and finally al reads longer than 18 bp was trimmed down to 18 bp by removing bases from the 3 end of the sequencing read. The number of unique reads was quantified and the top 10 most frequent reads are presented in FIG. 8 panel B-E. In FIG. 8 the reads have been reversed complemented in order to reads the original LNA oligo in a 5′->3′ sense manner.

### Example 8: NGS Sequencing of GalNac-LNA for QC

We illustrate here that sequencing of fully phosphorothioated LNA oligoes conjugated with a GalNac in the 5′ end for QC proposes is possible.

The following LNA oligo was used for sequencing.

LNA Mix:

Oligo 1:
(SEQ ID NO 20)
5′-GN2-C6caGCattggtatTCA

Capture Probes:

Capture probe 1 index 1:
(SEQ ID NO 12)
/5Phos/CCGCAATTGGCGTGATNNNNAGATCGGAAGAGCGTCGTGTAGT
CCGCATGTCGCGTGATAGGGATATCTTGCGGNNNNN/3AmMO/

Capture probe 1 RT primer:
(SEQ ID NO 16)
CTATCACGCGACATGCGG

First Ligation reaction:

2 ul Oligo 1 was mixed with 2 ul Capture probe 1 index 3 (10 uM)

Then mix was heated to 55 C then cool to 4 C.

16 ul Ligation mix was added to the tube:

Per 20 ul reaction: (2 ul T4 Ligation buffer, 6 ul PEG, 6 ul H2O, 2 ul T4 Ligase). Ligation:

2×(4 C; 2 min, 16 C; 20 min, 22 C 5 min, 30 C 1 min) 75 C; 10 min then hold on 4 C.
Gel electrophoresis:

To the above mentioned reaction an equal volume of 2× Novex® TBE-Urea Sample Buffer (Thermo Fisher Scientific) were added and the sample were heat denatured for 2 min at 95° C. and placed on ice. Fifteen μl of the prepared samples were loaded onto Novex® TBE-Urea Gels, 15%, 15 well (Thermo Fisher Scientific) and the electrophoresis was conducted for 75 min with constant voltage of 180 V. DNA was stained using SYBR Gold Nucleic Acid Gel Stain (Thermo Scientific) for 10 min. Gel was visualized with ChemiDoc Touch Imaging System(Bio Rad) on a Blue Tray

The band containing the ligation product between the capture probe and the oligo were cut from the gel. The Gel pieces were crunch and soaked in 500 ul TE buffer over night the extract the ligated oligoes. Following the soaking the ligated oligoes were washed and concentrated using Amicon Ultra 0.5 mL centrifugal MWCO 3 kDa filters. Finally the samples were concentrated to approximately 10 ul using a speedvac.
1.Strand synthesis and purification:

1. Strand synthesis was performed using the Vulcano2G DNA Polymerase in a 20 ul reaction:

4 ul: 5× Vulcano2G buffer

0.4 ul: Vulcano2G Polymerase

0.5 ul: dNTP (10 uM)

0.4 ul: Capture RT Primer (10 uM)

4 ul: Ligation template

H20 ad 20 ul

Using the following program on a thermocycler:

95 C; 3 min 10×(55 C; 5 min, 72 C; 1 min) then hold 4 C

The 1.strandsynthesis reaction were purified using the Monarch® PCR & DNA Cleanup Kit (New England Biolabs) using the manufactures Oligonucleotide Cleanup Protocol. Samples were eluted in 10 ul Elution buffer.
2.Ligation reaction:

Capture Probe 2
(SEQ ID NO 17)
/5Phos/CCGCAAGATCGGAAGAGCGGTTCAGCAGGAATGCATATGCTTG
CGGNNNNNN/3AmMO/

8 ul 1.strand synthesis reaction was mix with 2 ul Capture probe 2 (1 uM)

Then mix was heated to 55 C then cool to 4 C.

16 ul Ligation mix was added to each tube:

Per 20 ul reaction: (2 ul T4 Ligation buffer, 6 ul PEG, 6 ul H2O, 2 ul T4 Ligase). Ligation:

2×(4 C; 2 min, 16 C; 2 hours, 22 C 5 min) 75 C; 10 min then hold on 4 C.
PCR amplification of NGS Library:

NGS_PCR_primer1
(SEQ ID NO 18)
AATGATACGGCGACCACCGAGATCTACACTCTTTCCCTACACGACGCTCT
TCCGATCT
NGS_PCR_primer2
(SEQ ID NO 19)
CAAGCAGAAGACGGCATACGAGATCGGTCTCGGCATTCCTGCTGAACCGC
TCTTCCGATCTT

PCR amplification of the ligated 1.strand synthesis was performed with a phusion DNA polymerase using the NGS_PCR_primer1 and 2. These primers contain 5′ overhang compatible with illuminas TruSeq NGS protocol.

| | |
|---|---|
| 4 ul: | 5x HF Buffer |
| 0.4 ul: | dNTP (10 uM) |
| 1 ul: | NGS_PCR_primer1 (10 uM) |
| 1 ul: | NGS_PCR_primer2 (10 uM) |
| 0.2 ul: | Phusion DNA polymerase |
| 11.4 ul: | H₂O |
| 2 ul: | Sample from 2. Ligation reaction |

PCR cycling: 98 C; 30s, 15×(98 C; 15 s, 60 C; 20s, 72 C; 20s), 72 5 min then hold 4 C The PCR product was purified on a QIAquick PCR purification kit (Qiagen) according to manufactures instructions and eluted in 30 ul H₂O.
NGS setup:

The reaction were normalized to 10 nM and pooled with another NGS library containing a different barcoding system to create a 10 nM NGS library. This single reaction comprised approximately 10% of the total NGS library. The NGS library was prepared according to Illuminas Denature and Dilute Library Guide for the MiniSeq System. The library was sequenced on an Illumina miniSeq system using a MID output cassette. The sequencing was setup to generate fastq files use only read 1 and without indexes performing 151 cycles.
NGS data analysis:

The generated fastq files were imported into the CLC Genomics Workbench 10 software (Qiagen). The reads originating from the Capture Probes 1 index 1 was isolated and the remaining reading from capture probe 1 was trimmed away from the 5′end of the reads. Subsequently the sequence originating from the Capture Probe 2 was trimmed away from the 3′end leaving behind only the sequence inserted between the capture probes 1 and 2. Using awk command lines all reads shorter than 15 was then trimmed away, and finally al reads longer than 15 bp was trimmed down to 15 bp by removing bases from the 3 end of the sequencing read. The number of unique reads was quantified and the top 5 most frequent reads are presented in FIG. 9. In FIG. 9 the reads have been reversed complemented in order to reads the original LNA oligo in a 5′->3′ sense manner.

### Example 9: Comparing SuperScript III Reverse Transcriptase Vs Vulcano2G 1. Strand Synthesis Assay of LTT1

Crouzier et al. describes that Superscript III Reverse Transcriptase (RT) (Thermos Scientific) has the ability to read through LNA nucleotides (LNA-T and LNA-A) when reverse transcribing a RNA strand. The authors show that SuperScript III RT can incorporate nonconsecutive LNA-T's and LNA-As when reading a RNA template. The authors also show that Superscript III RT can reverse transcribe an RNA template containing 2 LNA A's and 2 LNA T's (nonconsecutive) using just normal dNTPs (Crouzier et al. 2012 FIG. 3 panel C lane 2).

Since SuperScript III RT has been shown to be able to reverse transcribe a an strand containing nonconsecutive LNAs in an RNA phosphodiester linked strand (Crouzier et al. 2012) we want to see if it is also able to read across consecutive LNAs in a DNA strand that also contains phosphorothioate backbone. Hence we perform 1 strand synthesis of the LTT1 template followed by 1.strand detection using EvaGreen ddPCR as described in Example 2. We compared the SuperScript III RT ability to create a copy of the LTT1 with Vulcano2G Polymerases ability. Reaction conditions for the 1.strand SuperScript III RT reaction was the same as described in Crouzier et al. 2012.

10 ul 1.Strand synthesis reactions:

All reaction contained (1 ul LTT1 (31 pM), 0.5 ul DCP1_primer1 (1 uM) and water add 10 ul. The enzymes were run with the buffer provided from the vendor.
a) 2 ul 5× First Strand Buffer, 1 ul MgCL₂ (50 mM), 0.75 ul DTT (100 mM), 0.75 ul dNTP (10 mM)
b) 2 ul 5× First Strand Buffer, 1 ul MgCL₂ (50 mM), 0.75 ul DTT (100 mM), 0.75 ul dNTP (10 mM), 0.5 SuperScript III RT enzyme
c) 2 ul 5× Vulcano2G Buffer, 1 ul dNTP(10 mM)
d) 2 ul 5× Vulcano2G Buffer, 1 ul dNTP(10 mM), 0.2 ul Vulcano2G

All components were added except the enzyme at the mixes were headed to 65 C 5 min then on ice 1 min before the RT enzyme was finally added. 1.Strand synthesis was done under 4 conditions.
a) 45 min 50 C, 5 min 80 C, hold 4 C
b) 5 min 50 C, hold 4 C
c) 3× cycles of (5 min 50 C, 30 sek 98 C) then hold 4 C
d) 5× cycles of (5 min 50 C, 30 sek 98 C) then hold 4 C

The cycling conditions in condition c and d were added since the Vulcano2G enzyme is thermostable, and hence multiple rounds of 1.strand synthesis can be done to increase sensitivity with this enzyme unlike Superscript III RT which is inactivated by higher temperatures needed to denature the doublestrand.

All 1.strand reactions were diluted 100× in water, and 2 ul sample was used as input for a normal QX200™ ddPCR™ EvaGreen Supermix PCR reaction as described in Example 2.

Results:

FIG. 10 panel A displays the fluoresce intensities of the droplets in the EvaGreen ddPCR reactions performed on the 1×45 min 1 strand synthesis reaction. We saw no sign of SuperScript III RT ability to make a 1.strand copy of LTT1 as the same number of positive droplets were also seen in the reaction without enzyme. The quantification of detected copies are show in FIG. 10 panel B displaying Vulcano2G far superior ability to make a 1 strand copy of the LTT1 template. FIG. 10 panel C displays the fluoresce intensities of the droplets in the EvaGreen ddPCR reactions performed on the reaction done with 1 3 or 5 rounds of 1. Strand synthesis reaction. The quantification of detected copies are show in FIG. 10 panel D displaying that Vulcano2G can perform several rounds of 1 strand synthesis reactions due to its thermos stability, which can be used to increase the detection of the LNA containing molecules. Again we saw no sign that superscript III RT can reverse transcribe the LTT1 template.

Ref:

Crouzier et al. (2012) Efficient Reverse Transcription Using Locked Nucleic Acid Nucleotides towards the Evolution of Nuclease Resistant RNA Aptamers. PLoS ONE April 2012, Volume 7, Issue 4, e35990

### Example 10: Enabling Ligation of a Capture Probe to the 5′ End of LNA Oligonucleotides

Objective of this example is to find conditions, which allow for covalently attaching a capture probe to the 5′ end of an LNA-containing, phosphorothioated oligonucleotide (LNA-oligonucleotide).

To prepare adapter-oligonucleotide mixes, 5.5 μl of 10 pM oligonucleotide L1 (table B) was mixed with 5.5 μl of 100 pM capture probe C1 or C2 or C3 or C4 or C5 (table a).

TABLE a
Oligonucleotides. All linkages are phosphodiesters, all nucleotides
are DNA nucleotides unless preceded by “r”, in which case they are
ribonucleotides. “/36-FAM/” indicates 3′ FAM group, /iSp18/
indicates 18-atom hexaethyleneglycol spacer, /3AmMO/ indicates a
3′Amino modifier, “5Cy5/” indicates 5′ Cy5 group. Nucleotide codes
are as per IUPAC nucleotide code.

| | | |
|---|---|---|
| C1 | /5Cy5/GTTCAGAGTTCTACAGTCCGACGATC | SEQ ID NO 95 |
| C2 | /56-FAM/NNNNNNGATCGTCG/iSp18/GTTCAGAGTTCTACAGTCCGACGATC | SEQ ID NO 96 |
| C3 | /56-FAM/NNNNNNGATCGTCG/iSp18/GTTCAGAGTTCTACAGTCCGACrGrArUrC | SEQ ID NO 97 |
| C4 | /56-FAM/rNrNrNrNrNrNrGrArUrCrGrUrCrG/iSp18/GTTCAGAGTTCTACAG | SEQ ID NO 98 |
| | TCCGACGATC | |
| C5 | /56-FAM/rNrNrNrNrNrNrGrArUrCrGrUrCrG/iSp18/GTTCAGAGTTCTACAG | SEQ ID NO 99 |
| | TCCGACrGrArUrC | |
| a4 | /5Phos/CTATCCAGCNNNTGGAATTCTCGGGTGCCAAGGA/iSp18/GCTGGATAGNN | SEQ ID NO 100 |
| | NNNN/36-FAM/ | |
| RTP | GCCTTGGCACCCGAGAATTCCA | SEQ ID NO 101 |
| RP1 | AATGATACGGCGACCACCGAGATCTACACGTTCAGAGTTCTACAGTCCGA | SEQ ID NO 102 |
| RP\|12 | CAAGCAGAAGACGGCATACGAGATTACAAGGTGACTGGAGTTCCTTGGCACCCGAGAAT | SEQ ID NO 103 |
| | TCCA | |
| RP\|27 | CAAGCAGAAGACGGCATACGAGATAGGAATGTGACTGGAGTTCCTTGGCACCCGAGAAT | SEQ ID NO 104 |
| | TCCA | |
| RP\|28 | CAAGCAGAAGACGGCATACGAGATCTTTTGGTGACTGGAGTTCCTTGGCACCCGAGAAT | SEQ ID NO 105 |
| | TCCA | |
| RP\|29 | CAAGCAGAAGACGGCATACGAGATTAGTTGGTGACTGGAGTTCCTTGGCACCCGAGAAT | SEQ ID NO 106 |
| | TCCA | |
| RP\|30 | CAAGCAGAAGACGGCATACGAGATCCGGTGGTGACTGGAGTTCCTTGGCACCCGAGAAT | SEQ ID NO 107 |
| | TCCA | |
| RP\|11 | CAAGCAGAAGACGGCATACGAGATGTAGCCGTGACTGGAGTTCCTTGGCACCCGAGAAT | SEQ ID NO 108 |
| | TCCA | |

TABLE B
Modified Oligonucleotides - All linkages
are phosphothioate. Upper-case nucleotides
are LNA nucleotides. Lower-case nucleotides
are DNA nucleotides. Upper-case “C”
nucleotides is a 5-methyl-cytidine.
“5′p” indicates 5′ phosphorylation.

| L1 | 5′pNNNnnnnnnnnnnNNN | SEQ ID NO |
|---|---|---|
| | | 109 |
| L2 | 5′pAGCattggtatTCA | SEQ ID NO |
| | | 110 |

Those capture probe—LNA-oligonucleotide mixtures were incubated for 5 minutes at 50° C. and placed on ice. Five master mixes were prepared, named M1, M2, M3, M4 and M5. For master mix M1, 4 volumes of 50% PEG 4000 were combined with 3 volumes of H2O and 1 volume of 10×T4 DNA Ligase Buffer (Thermo Fisher Scientific). For master mix M2, 4 volumes of 50% PEG 4000 were combined with 1 volume 10×T4 RNA Ligase Buffer (New England Biolabs), 1 volume of 1 mg/ml BSA, 1 volume of 10 mM ATP and 1 volume of T4 RNA Ligase (Thermo Fisher Scientific, cat. EL0021). For master mix M3, 4 volumes of 50% PEG 4000 were combined with 2 volumes of H2O, 1 volume of 10×T4 DNA Ligase Buffer (Thermo Fisher Scientific) and 1 volume of T4 DNA DNA Ligase HC (Thermo Fisher Scientific, cat. EL0013). For master mix M4, 4 volumes of 50% PEG 4000 were combined with 2 volumes of H2O, 1 volume of 10×T4 RNA Ligase 2 buffer (New England Biolabs) and 1 volume of T4 RNA Ligase 2 (New England Biolabs, cat. M0239S). For master mix M5, 2 volumes of 50% PEG 4000 were combined with 5 volumes of 2×T7 DNA Ligase buffer (New England Biolabs) and 1 volume of T7 DNA Ligase (New England Biolabs, cat. M0318). Each of the master mixes M1 to M5 was split into five 8 μl aliquots. Two μl of the capture probe—LNA-oligonucleotide mixtures were added to the master mix aliquots, thus creating 25 different combinations. Reactions were incubated in thermocycler: two cycles of (2 min at 37° C., 3 min at 30° C., 5 min at 22° C., 80 min at 16° C.), followed by addition of 2p1 of 2× Novex® TBE-Urea Sample Buffer (Thermo Fisher Scientific), incubation for 2 minutes at 95° C. and placing on ice.

To analyze the reactions, 7.5 μl of thus prepared samples were loaded onto Novex® TBE-Urea Gels, 15%, 15 well (Thermo Fisher Scientific) and the electrophoresis was conducted for 75 min with constant voltage of 180 V. Gel was visualized with ChemiDoc Touch Imaging System (Bio Rad) on a Blue Tray (FIG. 11). Following first visualization, the gels were stained with 1×SYBR Gold Nucleic Acid Gel Stain (Thermo Fisher Scientific) in 1× Novex TBE Running Buffer (Thermo Fisher Scientific) for 30 minutes and were again visualized with the same instrument (FIG. 12).

The experiment indicates that the most efficient ligation to the 5′ end of 5′ phosphorylated, phosphorothioated, LNA-containing oligonucleotide was achieved using C3 or C5 capture probes and T4 RNA Ligase or T4 RNA Ligase 2 enzymes (in any combination).

### Example 11: Ligation of Both 3′ and 5′ Capture Probes to the LNA Oligonucleotides

Capture probes and LNA-oligonucleotides were mixed according to table C.

TABLE C

| | | LNA- | Master | Master mix |
|---|---|---|---|---|
| Sample | Capture probe | oligonucleotide | mix | volume |
| 1 | a4 + C5 | H2O | M6 | 14 |
| 2 | a4 + C5 | L1 | M6 | 14 |
| 3 | a4 + C5 | L1 | M6 | 14 |
| 4 | a4 + C5 | L1 | M11 | 14 |
| 5 | a4 + C5 | L1 | M12 | 14 |
| 6 | a4 + C5 | L1 | M9 | 14 |
| 7 | C5 + H2O | L1 | M6 | 14 |
| 8 | a4 + H2O | L1 | M6 | 14 |
| 9 | H2O + H2O | L1 | M6 | 14 |
| 10 | C5 | H2O | M7 | 12 |
| 11 | a4 | H2O | M8 | 12 |
| 12 | C5 | L1 | M7 | 12 |
| 13 | a4 | L1 | M8 | 12 |
| 14 | a4 + C5 | L1 | M10 | 14 |
| 15 | a4 + C5 | L2 | M6 | 14 |

Each indicated capture probe was added as 2p1 of 20 pM. Each indicated LNA-oligonucleotide was added as 2 μl of 10 pM. For samples with “H2O”, for each occurrence of “H2O” in a table, B μl of H2O was added. Each of the samples was incubated for 5 minutes at 50° C. and placed on ice.

Master mix M6 was prepared by combining 3 volumes of 50% PEG 4000, 1 volume of H2O, 1 volume of 10×T4 DNA Ligase Buffer (Thermo Fisher Scientific), 1 volume of T4 DNA DNA Ligase HC (Thermo Fisher Scientific, cat. EL0013) and 1 volume of T4 RNA Ligase 2 (MCLAB, cat. T4RL2-200). Master mix M7 was prepared by combining 3 volumes of 50% PEG 4000, 1 volume of H2O, 1 volume of 10×T4 DNA Ligase Buffer (Thermo Fisher Scientific), and 1 volume of T4 RNA Ligase 2 (MCLAB, cat. T4RL2-200). Master mix M8 was prepared by combining 3 volumes of 50% PEG 4000, 1 volume of H2O, 1 volume of 10×T4 DNA Ligase Buffer (Thermo Fisher Scientific), and 1 volume of T4 DNA DNA Ligase HC (Thermo Fisher Scientific, cat. EL0013). Master mix M9 was prepared by combining 3 volumes of 50% PEG 4000, 3 volumes of H2O and 1 volume of 10×T4 DNA Ligase Buffer (Thermo Fisher Scientific). Master mix M10 was prepared by incubating master mix M6 for 10 minutes at 70° C. Master mix M11 was prepared by adding 2 μl of H2O to 12 μl of master mix M7. Master mix M12 was prepared by adding 2 μl of H2O to 12 μl of master mix M8. 12 μl or 14 μl of master mixes were added to prepared capture probe—LNA-oligonucleotide mixtures according to “Master mix volume” column in table C.

Samples (capture probe with LNA-oligonucleotide and appropriate master mix) were incubated in thermocycler: four cycles of (2 min at 37° C., 3 min at 30° C., 5 min at 22° C., 10 min at 16° C.), followed by incubating 10 min at 70° C. Following the thermal cycling, to samples 10 and 12, 2 μl of 20 pM capture probe a4 and 2 μl T4 DNA DNA Ligase HC was added. To samples 11 and 13, 2 μl of 20 pM capture probe C5 and 2 μl T4 RNA Ligase 2 was added. Samples 10, 11, 12 and 13 were incubated in thermocycler: four cycles of (2 min at 37° C., 3 min at 30° C., 5 min at 22° C., 10 min at 16° C.), followed by incubating 10 min at 70° C.

To analyze the reactions, 5 μl of each reaction was mixed with 5 μl of 2× Novex® TBE-Urea Sample Buffer (Thermo Fisher Scientific), incubated for 2 minutes at 95° C. and placed on ice. Thus prepared samples have been loaded onto Novex® TBE-Urea Gels, 15%, 15 well (Thermo Fisher Scientific) and the electrophoresis was conducted for 75 min with constant voltage of 180 V. Gel was visualized with ChemiDoc Touch Imaging System(Bio Rad) on a Blue Tray (FIG. 13).

The experiment indicates that to minimize the extent of unwanted direct ligation between 3′capture probe and 5′ capture probe, the preferred reaction setup is to first ligate the 5′ capture probe (C5) to the LNA-oligonucleotide, followed by ligating the 3′ capture probe (a4) (as in sample 12).

### Example 12 Preparation of Sequencing Library

Ligation of Capture Probes to LNA-Containing Oligonucleotides

Sample n1 was prepared by first mixing 5 μl of 20 pM capture probe C5 with 5 μl of 10 pM LNA-oligonucleotide L1 and was incubated for 5 minutes at 50° C. and placed on ice, followed by addition of 30 μl master mix M7 (as described in example 10). Sample was incubated in thermocycler four cycles of (2 min at 37° C., 3 min at 30° C., 5 min at 22° C., 10 min at 16° C.), followed by incubating 10 min at 70° C. 5 μl of 20 pM capture probe a4 and 5 μl T4 DNA DNA Ligase HC were added, followed by incubation in thermal cycler for 25 cycles of (2 min at 37° C., 3 min at 30° C., 5 min at 22° C., 10 min at 16° C., 10 min at 8° C.), followed by incubating 10 min at 70° C. Sample n2 was prepared in a similar way as sample n1, with a change that instead of LNA-oligonucleotide L1, LNA-oligonucleotide L2 was used. To check the reaction performance, 5 μl of samples n1 and n2 were mixed with 5 μl of 2× Novex® TBE-Urea Sample Buffer (Thermo Fisher Scientific), incubated for 2 minutes at 95° C. and placed on ice, loaded onto Novex® TBE-Urea Gels, 15%, 15 well (Thermo Fisher Scientific) and the electrophoresis was conducted with constant voltage of 180 V. Gel was visualized with ChemiDoc Touch Imaging System (Bio Rad) on a Blue Tray (FIG. 14). Products indicated by arrows on FIG. 14 are LNA-oligonucleotides covalently bound with both a4 and C5 capture probes.

First Strand Synthesis

Sequencing libraries were prepared with samples n1, n2 (as described above, ligation reactions were not purified) and samples 2, 10 and 12 (as described in example 10, ligation reactions were not purified). To 5 μl of each sample, 2 μl 10 pM RTP primer and 3 μl H2O were added, followed by 5 minute incubation at 65° C. and placing on ice. Master mix for the first strand synthesis was prepared by mixing 4 volumes of 5× PrimeScript buffer (Takara), 4 volumes H2O, 1 volume 10 mM dNTP and 1 volume PrimeScript enzyme (Takara, Code No. 2680A). 10 μl of this master mix was added to each of the samples, and they were incubated 30 min at 42° followed by 15 min at 50° C. and 15 min at 70° C.

PCR

PCR master mix for the PCR reaction was prepared by mixing 62 volumes of H2O, 10 volumes of 10× Phusion HF buffer, 10 volumes of 10 pM RP1 primer, 2 volumes and 1 volume of Phusion® High-Fidelity DNA Polymerase (New England Biolabs, cat. M0530). To 1 μl of the first strand synthesis reaction, 2 μl of the RPI12, RP127, RP128, RP129 or RP130 primer was added to samples 2, 10, 12, n1 or n2, respectively. 17 p1 of the PCR master mix was added to the first strand synthesis reaction-primer mix. Samples were cycled in a thermocycler with a program: 3 min at 98° C., 8 cycles of (80 seconds at 98° C., 30 seconds at 60° C., 15 seconds at 72° C.), 10 min at 72° C., followed by hold at 4° C. The PCR amplified samples were diluted 6× with H2O and assayed with Bioanalyzer DNA High Sensitivity kit (FIG. 15). Bioanalyzer traces display two predominant peaks, one for empty sequencing library (marked as E on the plots), derived from direct ligation of capture probes C5 and a4, and filled sequencing library (marked as F on the plots), which includes the sequence of the assayed LNA-oligonucleotide. Negative control sample was obtained by using H2O (instead of ligation reaction) as input for first strand synthesis, and was amplified in PCR using the same protocol as other samples, but with RPI11 primer (instead of RPIxx primers).

Sequencing

To 3.8 μl of 50 mM EDTA we added 10 μl, 0.48 μl, 4.52 μl, 6.54 μl 1.26 μl of PCR products obtained with primers RPI12, RP127, RP128, RP129, RPI30, respectively. Such obtained mixture was Ampure XP (Beckman Coulter, item number A63881) purified according to manufacturer's instructions. Sample was mixed with another Illumina-compatible sequencing library, and were sequenced with HiSeq Rapid V2 protocol (Illumina) with 50 bp single-end reads, including index reads.

### Example 13 Sequencing Data Analysis

Demultiplexed reads were first adapter-trimmed with cutadapt utility version 1.12 (described in http://dx.doi.orq/10.14806/ej.17.1.200), using command: “cutadapt-a CTATCCAGCNNNTGGAATTCTCGGGTGCCAAGGA-q 20-m 10-M 20 input.fastq”, where input.fastq.gz was replaced with an appropriate fastq file. Used cutadapt options ensure that only the reads that after trimming the adapter are of length between 10 to 20 nucleotides are kept. Statistics for adapter trimming are shown in table D.

TABLE D

| | | Total | After | After |
|---|---|---|---|---|
| Sample | Index | reads | cutadapt | cutadapt % |
| 2 | 12 | 11395093 | 4653848 | 41% |
| 10 | 27 | 619001 | 64156 | 10% |
| 12 | 28 | 6647678 | 4646007 | 70% |
| n1 | 29 | 13006844 | 7020142 | 54% |
| n2 | 30 | 3564400 | 926273 | 26% |

Finally, to assess the quality of sequencing we have analyzed for the consistence of obtained sequences with the characteristics of the input LNA-oligonucleotides. Input for samples 2, 12 and n1 was 16 mer LNA-oligonucleotide with randomized sequence (L1), input for sample n2 was 14 mer LNA-oligonucleotide with fixed sequence (L2), input for sample 10 was only H2O, and no oligonucleotide. Counting the occurrences of 14 mers and 16 mers in adapter-trimmed dataset, reveals enrichment for 14 mers for sample n2 and for 16 mers for samples 2, 10 and n1 (Table E).

TABLE E

| | | 14 mers | 16 mers | | | | | | |
|---|---|---|---|---|---|---|---|---|---|
| | | after | after | 14 mers | 16 mers | L2 | Unique | L2 | Unique |
| Sample | Index | cutadapt | cutadapt | % | % | match | 16 mers | % | % |
| 2 | 12 | 76696 | 3678617 | 2% | 79% | 495 | 03621115 | 1% | 98% |
| 10 | 27 | 1671 | 2903 | 3% | 5% | 5 | 1965 | 0% | 68% |
| 12 | 28 | 72777 | 3691428 | 2% | 79% | 393 | 3641931 | 1% | 99% |
| n1 | 29 | 120461 | 5469403 | 2% | 78% | 646 | 5378001 | 1% | 98% |
| n2 | 30 | 665311 | 5459 | 72% | 1% | 644073 | 1443 | 97% | 26% |

Remarkably, approximately 97% of 14 mers for sample n2 perfectly matches the sequence of the oligonucleotide L2. Moreover, for the samples for which the input was the randomized L1 oligonucleotide, more than 98% of 16 mers are uniquely represented.

### Example 14: In Vivo Conjugate Discovery

Data showing proof of principle that a library of various chemical moieties conjugated to DNA/PS oligonucleotides containing barcodes can be investigated for their liver enrichment in single animals using sequencing. Data are showing as a proof of principle that a GalNAc-conjugated oligonucleotide (SEQ ID 22) is enriched in the liver 4 h after subcutaneous injection compared to naked oligo (SEQ ID 35). Data also identifies, that SEQ ID 26 are enriched in the liver 4 h after subcutaneous injection compared to the naked oligo SEQ ID 37. A solution of 5 pM each of the oligos (table below) was injected subcutaneously into C57BL/6J mice (n=3) at a dose of 0.25 mL pr mouse and liver were harvested 4 h after injection.

Table. Library of barcoded oligos with conjugations. Barcodes for identification are shown in bold.

Conjugations are shown as either chemical structures or in words. In the chemical structures the wavy line represents the covalent bond to the oligonucleotide, suitable the 5′ terminus optionally via a linker such as a C6 alkyl linker group.

TABLE 13

| | Oligonucleotide for | |
|---|---|---|
| Compound | Conjugation - All PS linkages | Conjugation |
| SEQ ID | C6-Aminolink- T GTC TAG | GalNAc (A) |
| NO NO 22 | AATGC GCA CGT C -3′ | |
| | (SEQ ID NO 22) | |
| SEQ ID NO 23 | C6-Aminolink- T GTC TAG AGCCT GCA CGT C -3′ (SEQ ID NO 23) | 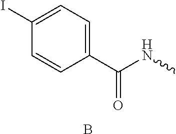 |
| SEQ ID NO 24 | C6-Aminolink- T GTC TAG CATGT GCA CGT C -3′ (SEQ ID NO 24) | 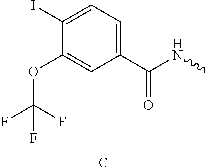 |
| SEQ ID NO 25 | C6-Aminolink- T GTC TAG ATGTA GCA CGT C -3′ (SEQ ID NO 25) | 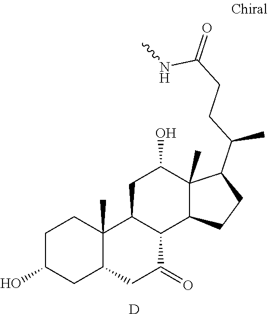 |
| SEQ ID NO 26 | C6-Aminolink- T GTC TAG CGTAC GCA CGT C -3′ (SEQ ID NO 26) | 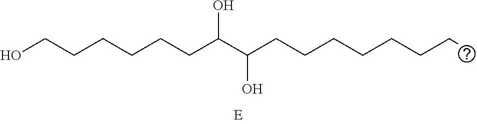 [SMILES: CCCCCCCCC(O)C(O)CCCCCCO] |
| SEQ ID NO 27 | C6-Aminolink- T GTC TAG GAATG GCA CGT C -3′ (SEQ ID NO 27) | 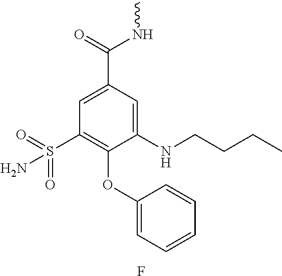 |
| SEQ ID NO 28 | C6-Aminolink- T GTC TAG GTAAT GCA CGT C -3′ (SEQ ID NO 28) | 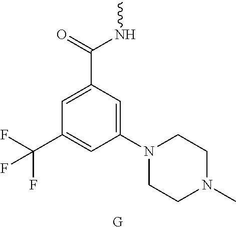 |
| SEQ ID NO 29 | C6-Aminolink- T GTC TAG GCGTG GCA CGT C -3′ (SEQ ID NO 29) | 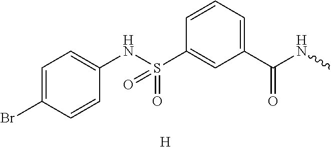 |
| SEQ IN NO 30 | C6-Aminolink- T GTC TAG ACCTA GCA CGT C -3′ (SEQ ID NO 30) | 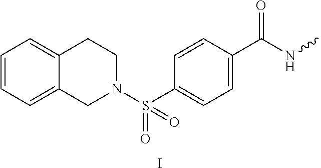 |
| SEQ ID NO 31 | C6-Aminolink- T GTC TAG CAACT GCA CGT C -3′ (SEQ ID NO 31) | 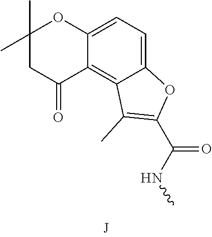 |
| SEQ ID NO 32 | C6-Aminolink- T GTC TAG ATTCA GCA CGT C -3′ (SEQ ID NO 32) | 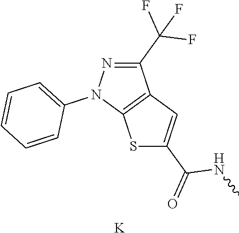 |
| SEQ ID NO 33 | C6-Aminolink- T GTC TAG GTCCT GCA CGT C -3′ (SEQ ID NO 33) | 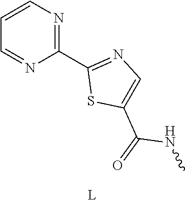 |
| SEQ ID NO 34 | C6-Aminolink- T GTC TAG ACTTG GCA CGT C -3′ (SEQ ID NO 34) | 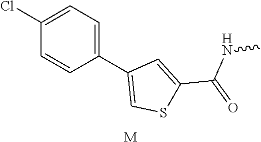 |
| SEQ ID | C6-Aminolink- T GTC TAG | no acid |
| NO 35 | ACGCT GCA CGT C -3′ | |
| | (SEQ ID NO 35) | |

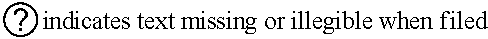

4 h after in vivo injection, liver tissue samples (100 mg) were homogenized using Tissue Lyzer (Qiagen) in a 400 μL buffer containing 0.1 M CaCl₂), 0.1 M Tris pH 8.0 and 1% NP-40. In parallel, 10 μL of the solution containing 5 pM each oligo was spiked into ex vivo liver samples (100 mg, n=3) that were homogenized as described above. After adding 25 μL Proteinase K (Sigma), samples were incubated overnight at 50° C. 1 μL RNAseif (New England Biolabs) and 1 μL DNase I (Qiagen) was then added and samples were incubated for 1 h at 37° C., inactivation of nucleases was done by incubation at 99° C. for 15 min. Samples were spun down 10000 g for 10 min, and supernatant were washed three times using Amicon Ultra 0.5 mL centrifugal MWCO 3 kDa filters, and washing was performed by adding approximately 400 μL of distilled water at each washing step. 2 μL of the remaining Supernatant (of approximately 50 μL) were used in the first ligation reaction containing 1 μL Capture probe 1 (10 pM), 2 μL T4 ligase buffer, 6 μL PEG, 1 μL T4 Ligase (ThermoFisher Scientific) and 8 μL distilled water. Each samples were ligated to a specific capture probe with a specific index sequence for later identification of sequences from each individual sample (see table below).

TABLE 14
Library of Capture probe 1. Index for
identification is shown in bold

| Capture probe | Sequence |
|---|---|
| F1_SeqCapP1_v6_I1 | /5Phos/CCG CAA GTG GCG TGA TNN NNA GAT CGG AAG |
| | AGC GTC GTG TAG TCC GCA TGT CGC GTG ATA GGG ATA |
| | TCT TGC GGG ACG TG/3ddC/ (SEQ ID NO 36) |
| F1_CapP1_v6_I2 | /5Phos/CCG CAA GTG GAC ATC GNN NNA GAT CGG AAG |
| | AGC GTC GTG TAG TCC GCA TGT CGC GTG ATA GGG ATA |
| | TCT TGC GGG ACG TG/3ddC/ (SEQ ID NO 37) |
| F1_CapP1_v6_I3 | /5Phos/CCG CAA GTG GGC CTA ANN NNA GAT CGG AAG |
| | AGC GTC GTG TAG TCC GCA TGT CGC GTG ATA GGG ATA |
| | TCT TGC GGG ACG TG/3ddC/ (SEQ ID NO 38) |
| F1_CapP1_v6_I4 | /5Phos/CCG CAA GTG GTG GTC ANN NNA GAT CGG AAG |
| | AGC GTC GTG TAG TCC GCA TGT CGC GTG ATA GGG ATA |
| | TCT TGC GGG ACG TG/3ddC/ (SEQ ID NO 39) |
| F1_CapP1_v6_I5 | /5Phos/CCG CAA GTG GCA CTG TNN NNA GAT CGG AAG |
| | AGC GTC GTG TAG TCC GCA TGT CGC GTG ATA GGG ATA |
| | TCT TGC GGG ACG TG/3ddC/ (SEQ ID NO 40) |
| F1_CapP1_v6_I6 | /5Phos/CCG CAA GTG GAT TGG CNN NNA GAT CGG AAG |
| | AGC GTC GTG TAG TCC GCA TGT CGC GTG ATA GGG ATA |
| | TCT TGC GGG ACG TG/3ddC/ (SEQ ID NO 41) |

After incubation for 1 h at 16° C. and inactivation for 15 min at 75° C., an equal volume of 2× Novex® TBE-Urea Sample Buffer (Thermo Fisher Scientific) was added to this first ligation product reaction and the sample were heat denatured for 2 min at 95° C. and placed on ice. Fifteen μl of the prepared samples were loaded onto Novex® TBE-Urea Gels, 15%, 15 well (Thermo Fisher Scientific) and the electrophoresis was conducted for 75 min with constant voltage of 180 V. DNA was stained using SYBR Gold Nucleic Acid Gel Stain (Thermo Scientific) for 10 min. Gel was visualized with ChemiDoc Touch Imaging System (Bio Rad) on a Blue Tray. The bands containing the ligation product of the capture probe ligated to the oligonucleotides were cut out from the gel. The gel pieces were then crunched and soaked in 500p1 distilled water and left overnight at 4° C. to extract the ligated oligonucleotides. The extracted ligated oligonucleotides were then washed 3× by adding approximately 400 μL of distilled water at each washing step using Amicon Ultra 0.5 mL centrifugal MWCO 3 kDa filters. After the final wash the concentrated oligonucleotides was then used in the first strand synthesis reaction. First strand synthesis was performed using 4 μL of the homogenized ligated gel input, 4 μL 5× Volcano2G buffer (MyPols), 0.4 μL of First strand primer 1 pM, 0.5 μL 10 mM dNTP, 0.4 μL Volcano2G Polymerase (MyPols), and 10.7 μL distilled water. PCR conditions were 95° C. for 3 min, followed by 15 cycles of 95° C. for 30 s, 55° C. for 5 min, and 72° C. for 1 min.

| First Strand | |
|---|---|
| primer | Sequence |
| RT_Primer | 5CCCTATGACGCGACATGCGGA3 (SEQ ID NO 42) |
| SeqCapP1_V6 | |

The first strand PCR product was purified using the Monarch PCR and DNA clean up kit (New England Biolabs) according to manufactorer's instruction and eluated in 10 μL elution buffer. 8 μL of the eluted first strand product was then ligated to 2 μL Capture probe 2 (100 nM) and heated to 60° C. for 5 min followed by a slow (0.1° C./s) decline in temperature to 4° C. Then 10 μL consisting of 2 μL T4 ligase buffer, 6 μL PEG, 1 μL T4 Ligase (ThermoFisher Scientific) and 1 μL distilled water was added.

| Capture probe 2 | Sequence |
|---|---|
| F1_SeqCapP2_v6 | /5Phos/CCG CAA GGA GAT CGG |
| | AAG AGC ACA CGT CTG AAC TCC |
| | ATA TGC TTG CGG CGT CTA C/ |
| | 3AmMO/ (SEQ ID NO 43) |

After incubation for 1 h at 16° C. and inactivation for 15 min at 75° C., 2 μL of the ligation reaction was applied to the PCR reaction containing 4 μL 5×HF buffer (New England Biolabs), 0.4 μL 10 mM dNTP, 1 μL 10 pM Forward Seq primer (PE1) and 1 μL 10 pM Reverse Seq primer (PE2V2), 0.2 μL Phusion DNA polymerase (New England Biolabs) and 11.4 μL distilled water. PCR conditions were 98° C. for 30 s, followed by 20 cycles of 98° C. for 15s, 60° C. for 20s, and 72° C. for 20 s, and finally 72° C. for 5 min. PCR product was purified using Qiaquick PCR purification kit (Qiagen) according to manufactorer's instructions and eluated in 30 μL distilled water.

| Capture | |
|---|---|
| probe | |
| 2 | Sequence |
| PE1 | 5AATGATACGGCGACCACCGAGATCTACACTCTTTCCCTA |
| | CACGACGCTCTTCCGATCT3 |
| | (SEQ ID NO 44) |
| PE2V2 | CAAGCAGAAGACGGCATACGAGATCGGTCTCGGCATTCCT |
| | GCTGAACCGCTCTTCCGATCT |
| | (SEQ ID NO 45) |

The PCR product was sequenced according to the protocol from Illumina MiniSeq System (Illumina). By using the software CLC Genomics Workbench, 11.0.1 (Qiagen) number of reads for the different Barcodes were identified and relative abundance of the barcodes were calculated. Finally, the ratios of the in vivo liver relative abundances to the ex vivo spike in liver samples relative abundances were calculated.

TABLE 15
4 h liver samples number of reads. and relative abundance for each sample (n = 3).

| | Liver Spike in | | | | | | Liver in vivo | | | | | |
|---|---|---|---|---|---|---|---|---|---|---|---|---|
| | Index | | Index | | Index | | Index | | Index | | Index | |
| Compound | 1 | % | 2 | % | 3 | % | 4 | % | 5 | % | 6 | % |
| SEQ ID | 47047 | 27.5 | 118134 | 29.4 | 93326 | 28.8 | 57631 | 57.6 | 21319 | 45.6 | 15343 | 46.5 |
| NO 22 | | | | | | | | | | | | |
| SEQ ID | 4762 | 2.8 | 13529 | 3.4 | 9242 | 2.9 | 787 | 0.8 | 428 | 0.9 | 347 | 1.1 |
| NO 23 | | | | | | | | | | | | |
| SEQ ID | 1221 | 0.7 | 2701 | 0.7 | 2167 | 0.7 | 447 | 0.4 | 200 | 0.4 | 269 | 0.8 |
| NO 24 | | | | | | | | | | | | |
| SEQ ID | 11869 | 6.9 | 27492 | 6.8 | 20992 | 6.5 | 4868 | 4.9 | 1510 | 3.2 | 847 | 2.6 |
| NO 25 | | | | | | | | | | | | |
| SEQ ID | 13665 | 8.0 | 35678 | 8.9 | 27137 | 8.4 | 11667 | 11.7 | 7017 | 15.0 | 5972 | 18.1 |
| NO 26 | | | | | | | | | | | | |
| SEQ ID | 1893 | 1.1 | 3758 | 0.9 | 2233 | 0.7 | 538 | 0.5 | 349 | 0.7 | 310 | 0.9 |
| NO 27 | | | | | | | | | | | | |
| SEQ ID | 13244 | 7.7 | 31129 | 7.7 | 23849 | 7.4 | 5067 | 5.1 | 1535 | 3.3 | 1643 | 5.0 |
| NO 28 | | | | | | | | | | | | |
| SEQ ID | 1983 | 1.2 | 3716 | 0.9 | 2962 | 0.9 | 590 | 0.6 | 378 | 0.8 | 407 | 1.2 |
| NO 29 | | | | | | | | | | | | |
| SEQ ID | 4405 | 2.6 | 12758 | 3.2 | 7608 | 2.3 | 1561 | 1.6 | 799 | 1.7 | 714 | 2.2 |
| NO 30 | | | | | | | | | | | | |
| SEQ ID | 7164 | 4.2 | 16922 | 4.2 | 10805 | 3.3 | 2141 | 2.1 | 815 | 1.7 | 949 | 2.9 |
| NO 31 | | | | | | | | | | | | |
| SEQ ID | 1306 | 0.8 | 3780 | 0.9 | 2269 | 0.7 | 497 | 0.5 | 283 | 0.6 | 192 | 0.6 |
| NO 32 | | | | | | | | | | | | |
| SEQ ID | 11707 | 6.8 | 28659 | 7.1 | 24125 | 7.4 | 4098 | 4.1 | 1935 | 4.1 | 2269 | 6.9 |
| NO 33 | | | | | | | | | | | | |
| SEQ ID | 4545 | 2.7 | 9787 | 2.4 | 7724 | 2.4 | 1323 | 1.3 | 750 | 1.6 | 613 | 1.9 |
| NO 34 | | | | | | | | | | | | |
| SEQ ID | 46353 | 27.1 | 94060 | 23.4 | 89553 | 27.6 | 8854 | 8.8 | 9415 | 20.1 | 3143 | 9.5 |
| NO 35 | | | | | | | | | | | | |
| Total | 171164 | | 402103 | | 323992 | | 100069 | | 46733 | | 33018 | |
| reads | | | | | | | | | | | | |

| | | | | | in vivo |
|---|---|---|---|---|---|
| | | | | | ratio |
| | Liver Spike in | | Liver in vivo | | relative |
| | abundance | standard | abundance | standard | to liver |
| Compound | (%) | dev | (%) | dev | spike in |
| SEQ ID NO 22 | 28.6 | 1.0 | 49.9 | 6.7 | 1.7 |
| SEQ ID NO 23 | 3.0 | 0.3 | 0.9 | 0.1 | 0.3 |
| SEQ ID NO 24 | 0.7 | 0.0 | 0.6 | 0.2 | 0.8 |
| SEQ ID NO 25 | 6.8 | 0.2 | 3.6 | 1.2 | 0.5 |
| SEQ ID NO 26 | 8.4 | 0.4 | 14.9 | 3.2 | 1.8 |
| SEQ ID NO 27 | 0.9 | 0.2 | 0.7 | 0.2 | 0.8 |
| SEQ ID NO 28 | 7.6 | 0.2 | 4.4 | 1.0 | 0.6 |
| SEQ ID NO 29 | 1.0 | 0.1 | 0.9 | 0.3 | 0.9 |
| SEQ ID NO 30 | 2.7 | 0.4 | 1.8 | 0.3 | 0.7 |
| SEQ ID NO 31 | 3.9 | 0.5 | 2.3 | 0.6 | 0.6 |
| SEQ ID NO 32 | 0.8 | 0.1 | 0.6 | 0.1 | 0.7 |
| SEQ ID NO 33 | 7.1 | 0.3 | 5.0 | 1.6 | 0.7 |
| SEQ ID NO 34 | 2.5 | 0.1 | 1.6 | 0.3 | 0.6 |
| SEQ ID NO 35 | 26.0 | 2.3 | 12.8 | 6.3 | 0.5 |

FIG. 16 shows the fold liver enrichment relative to unconjugated oligonucleotide (SEQ ID 35) 4 h after subcutaneous injection. GalNAc conjugated oligonucleotide (SEQ ID 22) as well as SEQ ID 26 show 3.5-fold liver enrichment compared to the unconjugated oligonucleotide (SEQ ID 35).

### Example 15

Data showing proof of principle that a library of various chemical moieties conjugated to DNA/PS oligonucleotides containing barcodes can be investigated for their plasma retention in single animals using sequencing. Data showing as proof of principle that an albumin binding 016 palmitate conjugated to an oligonucleotide is enriched in plasma 4 h after subcutaneous injection. Data also identifies that a GalNAc conjugated oligonucleotide is removed from circulation compared to a naked oligonucleotide.

A solution of 5 pM each of the oligonucleotides (see table below) in PBS was injected subcutaneously into C57BL/6J mice 8 (n=3) at a dose of 0.25 mL pr mouse and plasma samples were taken 4 h after injection.

TABLE
Library of barcoded oligonucleotides with
conjugations. Barcodes for identification
are shown in bold.
Compounds used are shown as the conjugates
in Table 13 above, and further included
Compound SEQ ID NO 46:
Oligonucleotide for conjugation - All

| Compound | PS linkages | Conjugations |
|---|---|---|
| SEQ ID NO | C6-Aminolink- T GTC TAG | Palmitate |
| 46 | TGCCA GCA CGT C-3′ | |
| | (SEQ ID NO 46) | |

4 h after injection, 10 μL plasma samples were homogenized in 240 μL RIPA buffer (Pierce) using Tissue Lyzer (Qiagen). In parallel, 10 μL of the solution containing 5 pM each oligos was spiked into 10 μL ex vivo plasma samples and samples were homogenized in 240 μL RIPA buffer as described above. 2 μL of the homogenized plasma RIPA solution was then used in the first ligation reaction containing 1 μL capture probe 1 (1 nM), 2 μL T4 ligase buffer, 6 μL PEG, 1 μL T4 Ligase (ThermoFisher Scientific) and 8 μL distilled water. Each samples were ligated to a specific capture probe 1 with a specific index sequence for later identification of each individual sample.

TABLE 16
Library of Capture probe 1. Index for
identification is shown in bold

| Capture probe | Sequence |
|---|---|
| F1_SeqCapP1_v6_I1 | /5Phos/CCG CAA GTG GCG TGA TNN NNA GAT CGG AAG |
| | AGC GTC GTG TAG TCC GCA TGT CGC GTG ATA GGG ATA |
| | TCT TGC GGG ACG TG/3ddC/ (SEQ ID NO 36) |
| F1_CapP1_v6_I2 | /5Phos/CCG CAA GTG GAC ATC GNN NNA GAT CGG AAG |
| | /AGC GTC GTG TAG TCC GCA TGT CGC GTG ATA GGG ATA |
| | TCT TGC GGG ACG TG/3ddC/ (SEQ ID NO 37) |
| F1_CapP1_vy6_I3 | /5Phos/CCG CAA GTG GGC CTA ANN NNA GAT CGG AAG |
| | AGC GTC GTG TAG TCC GCA TGT CGC GTG ATA GGG ATA |
| | TCT TGC GGG ACG TG/3ddC/ (SEQ ID NO 38) |
| F1_CapP1_v6_I4 | /5Phos/CCG CAA GTG GTG GTC ANN NNA GAT CGG AAG |
| | AGC GTC GTG TAG TCC GCA TGT CGC GTG ATA GGG ATA |
| | TCT TGC GGG ACG TG/3ddC/ (SEQ ID NO 39) |
| F1_CapP1_v6_I5 | /5Phos/CCG CAA GTG GCA CTG TNN NNA GAT CGG AAG |
| | AGC GTC GTG TAG TCC GCA TGT CGC GTG ATA GGG ATA |
| | TCT TGC GGG ACG TG/3ddC/ (SEQ ID NO 40) |
| F1_CapP1_v6_I6 | /5Phos/CCG CAA GTG GAT TGG CNN NNA GAT CGG AAG |
| | AGC GTC GTG TAG TCC GCA TGT CGC GTG ATA GGG ATA |
| | TCT TGC GGG ACG TG/3ddC/ (SEQ ID NO 41) |

After incubation for 1 h at 16° C. and inactivation for 15 min at 75° C., 2p1 of the ligation reaction was used for first Strand synthesis containing 2 μL of the ligation reaction, 4 μL 5× Volcano buffer (MyPols), 0.4 μL of first strand primer 1 pM, 0.5 μL 10 mM dNTP, 0.4 μL Volcano PG (MyPols), and 12.7 μL distilled water. PCR conditions were 95° C. for 3 min, followed by 15 cycles of 95° C. for 30 s, 55° C. for 30 min, and 72° C. for 1 min.

| First Strand primer | Sequence |
|---|---|
| RT_Primer SeqCapP1_V6 | 5CCCTATGACGCGACATGC |
| | GGA (SEQ ID NO 42) |

All the first strand PCRs product were pooled and 50 μL of the pooled first Strand PCR products were purified using the Monarch PCR and DNA clean up kit (New England Biolabs) according to manufactorer's instruction and eluated in 10 μL elution buffer. 8 μL of the eluted first strand product was then ligated to 2 μL Capture probe 2 (100 nM) and heated to 60° C. for 5 min followed by a slow (0.1° C./s) decline in temperature to 4° C. Then 10 μL consisting of 2 μL T4 ligase buffer, 6 μL PEG, 1 μL T4 Ligase (ThermoFisher Scientific) and 1 μL distilled water was added.

| Capture probe 2 | Sequence |
|---|---|
| F1_SeqCapP2_v6 | /5Phos/CCG CAA GGA GAT CGG |
| | AAG AGC ACA CGT CTG AAC TCC |
| | ATA TGC TTG CGG CGT CTA C/ |
| | 3AmMO/ (SEQ ID NO 43) |

After incubation for 1 h at 16° C. and inactivation for 15 min at 75° C., 2 μL of the ligation reaction was applied to the PCR reaction containing 4 μL 5×HF buffer, 0.4 μL 10 mM dNTP, 1 μL 10 pM Forward Seq primer (PE1) and 1 μL 10 pM Reverse Seq primer (PE2V2), 0.2 μL Phusion DNA polymerase (New England Biolabs) and 11.4 μL distilled water. PCR conditions were 98° C. for 30 s, followed by 20 cycles of 98° C. for 15s, 60° C. for 20s, and 72° C. for 20 s, and finally 72° C. for 5 min.

| PCR | |
|---|---|
| Primers | Sequence |
| PE1 | 5AATGATACGGCGACCACCGAGATCTACACTCTTTCCC |
| | TACACGACGCTCTTCCGATCT3 (SEQ ID NO 44) |
| PE2V2 | CAAGCAGAAGACGGCATACGAGATCGGTCTCGGCATTC |
| | CTGCTGAACCGCTCTTCCGATCT (SEQ ID NO 45) |

PCR product was purified using Qiaquick PCR purification kit (Qiagen) according to manufactorer's instructions and eluated in 30 μL distilled water. The PCR product was sequenced according to the protocol from Illumina MiniSeq System (Illumina). By using the software CLC Genomics Workbench, 11.0.1. number of reads for the different Barcodes were identified and relative number of barcodes were calculated. Finally, the ratios of the in vivo plasma relative abundances relative to the ex vivo spike in plasma samples were calculated.

TABLE 17
4 h plasma samples number of reads. and relative abundance for each sample (n = 3).

| | Plasma spike in | | | | | | In vivo Plasma | | | | | |
|---|---|---|---|---|---|---|---|---|---|---|---|---|
| | Index | | Index | | Index | | Index | | Index | | Index | |
| Compound | 1 | % | 2 | % | 3 | % | 4 | % | 5 | % | 6 | % |
| SEQ ID | 32047 | 33.0 | 221150 | 32.1 | 10388 | 25.9 | 352 | 1.8 | 78 | 3.6 | 176 | 2.3 |
| NO 22 | | | | | | | | | | | | |
| SEQ ID | 4909 | 5.1 | 32594 | 4.7 | 2377 | 5.9 | 2974 | 15.2 | 225 | 10.4 | 847 | 11.0 |
| NO 23 | | | | | | | | | | | | |
| SEQ ID | 2234 | 2.3 | 16392 | 2.4 | 1355 | 3.4 | 2077 | 10.6 | 140 | 6.5 | 501 | 6.5 |
| NO 24 | | | | | | | | | | | | |
| SEQ ID | 7658 | 7.9 | 51606 | 7.5 | 3712 | 9.2 | 869 | 4.4 | 154 | 7.1 | 485 | 6.3 |
| NO 25 | | | | | | | | | | | | |
| SEQ ID | 2963 | 3.1 | 20503 | 3.0 | 786 | 2.0 | 1144 | 5.8 | 119 | 5.5 | 563 | 7.3 |
| NO 26 | | | | | | | | | | | | |
| SEQ ID | 5475 | 5.6 | 45554 | 6.6 | 3008 | 7.5 | 1808 | 9.2 | 286 | 13.2 | 609 | 7.9 |
| NO 27 | | | | | | | | | | | | |
| SEQ ID | 10723 | 11.0 | 73483 | 10.7 | 5623 | 14.0 | 1125 | 5.7 | 189 | 8.7 | 614 | 8.0 |
| NO 28 | | | | | | | | | | | | |
| SEQ ID | 1103 | 1.1 | 8985 | 1.3 | 405 | 1.0 | 644 | 3.3 | 112 | 5.2 | 286 | 3.7 |
| NO 29 | | | | | | | | | | | | |
| SEQ ID | 5285 | 5.4 | 43510 | 6.3 | 2494 | 6.2 | 1315 | 6.7 | 220 | 10.1 | 725 | 9.5 |
| NO 30 | | | | | | | | | | | | |
| SEQ ID | 2514 | 2.6 | 23191 | 3.4 | 1217 | 3.0 | 1010 | 5.2 | 65 | 3.0 | 411 | 5.4 |
| NO 31 | | | | | | | | | | | | |
| SEQ ID | 6247 | 6.4 | 42786 | 6.2 | 2963 | 7.4 | 1221 | 6.2 | 168 | 7.7 | 482 | 6.3 |
| NO 32 | | | | | | | | | | | | |
| SEQ ID | 7631 | 7.9 | 49005 | 7.1 | 2929 | 7.3 | 1146 | 5.8 | 94 | 4.3 | 733 | 9.6 |
| NO 33 | | | | | | | | | | | | |
| SEQ ID | 5920 | 6.1 | 39661 | 5.8 | 2067 | 5.1 | 1104 | 5.6 | 114 | 5.3 | 614 | 8.0 |
| NO 34 | | | | | | | | | | | | |
| SEQ ID | 1496 | 1.5 | 12203 | 1.8 | 344 | 0.9 | 169 | 0.9 | 11 | 0.5 | 110 | 1.4 |
| NO 35 | | | | | | | | | | | | |
| SEQ ID | 855 | 0.9 | 9092 | 1.3 | 496 | 1.2 | 2634 | 13.4 | 195 | 9.0 | 512 | 6.7 |
| NO 44 | | | | | | | | | | | | |
| Total | 97060 | | 689715 | | 40164 | | 19592 | | 2170 | | 7668 | |
| Reads | | | | | | | | | | | | |

TABLE 18
4 h plasma samples relative abundance of reads (average and
standard deviations) and in vivo ratio relative to plasma spike in.

| | | | | | ratio in |
|---|---|---|---|---|---|
| | Spike in plasma | | In vivo plasma | | vivo/ |
| | Abundance | Standard | Abundance | Standard | spike |
| Compound | % | deviation | % | deviation | in |
| SEQ ID NO 22 | 30.3 | 3.9 | 2.6 | 0.9 | 0.1 |
| SEQ ID NO 23 | 5.2 | 0.6 | 12.2 | 2.6 | 2.3 |
| SEQ ID NO 24 | 2.7 | 0.6 | 7.9 | 2.4 | 2.9 |
| SEQ ID NO 25 | 8.2 | 0.9 | 6.0 | 1.4 | 0.7 |
| SEQ ID NO 26 | 2.7 | 0.6 | 6.2 | 1.0 | 2.3 |
| SEQ ID NO 27 | 6.6 | 0.9 | 10.1 | 2.7 | 1.5 |
| SEQ ID NO 28 | 11.9 | 1.8 | 7.5 | 1.6 | 0.6 |
| SEQ ID NO 29 | 1.1 | 0.1 | 4.1 | 1.0 | 3.5 |
| SEQ ID NO 30 | 6.0 | 0.5 | 8.8 | 1.8 | 1.5 |
| SEQ ID NO 31 | 3.0 | 0.4 | 4.5 | 1.3 | 1.5 |
| SEQ ID NO 32 | 6.7 | 0.6 | 6.8 | 0.9 | 1.0 |
| SEQ ID NO 33 | 7.4 | 0.4 | 6.6 | 2.7 | 0.9 |
| SEQ ID NO 34 | 5.7 | 0.5 | 6.3 | 1.5 | 1.1 |
| SEQ ID NO 35 | 1.4 | 0.5 | 0.9 | 0.5 | 0.7 |
| SEQ ID NO 44 | 1.1 | 0.2 | 9.7 | 3.4 | 8.5 |

FIG. 17 shows plasma enrichment relative to unconjugated oligo compound SEQ ID 35, 4 h after subcutaneous injection. Oligonucleotide with 016 fatty acid conjugation (SEQ ID 46) showed 12.5-fold plasma abundance compared to Naked oligonucleotide SEQ ID 35.

GalNAc conjugated oligonucleotide (SEQ ID 22) showed depletion from plasma.

### Example 16

Data showing proof of principle that a library of various chemical moieties conjugated to LNA/DNA/PS containing oligonucleotides containing barcodes can be investigated for tissue delivery properties in multiple tissues in single animals using sequencing. Data also identifies, that cholesterol conjugated (SEQ ID 49 and 50) and tocopherol conjugated oligonucleotides (SEQ ID 60 and 61) are enriched in the liver and are reduced in the kidney 3 days after iv injection. Data also identifies that a Bile amine conjugation (SEQ ID 51 and 52) increases oligonucleotide content in Pancreas.

A library of 15 barcoded oligonucleotides (table below) was injected intravenously into C57BL/6J mice (n=2) at a dose (all ollgonucleotides) of 10 mg/kg. The following organs were harvested and analysed 3 days later: adipose tissue, cortex, eye, femur, heart, ilium, kidney, liver, lung, lymph node, pancreas, serum, spinal cord spleen, and stomach.

TABLE 19
Library of barcoded oligos with conjugations.
Barcodes for identification are shown in bold.
Conjugations are shown in wording. LNA
nucleotides are shown as capital letters.

| | Oligo |
|---|---|
| Compound | for Conjugation - All PS linkages |
| SEQ ID NO 47 | 5′-Stearyl-CgTctacatccacccacgTC |
| SEQ ID NO 48 | 5′-Stearyl-CgTctacgcttgtccacgTC |
| SEQ ID NO 49 | 5′-Cholesterol-CgTctacggacttccacgTC |
| SEQ ID NO 50 | 5′-Cholesterol-CgTctacaagtccccacgTC |
| SEQ ID NO 51 | 5′-Bile amine-CgTctactctctaccacgTC |
| SEQ ID NO 52 | 5′-Bile amine-CgTctacctctcgccacgTC |
| SEQ ID NO 53 | 5′-Bile alcohol-CgTctacagttcaccacgTC |
| SEQ ID NO 54 | 5′-Bile alcohol-CgTctacgacctgccacgTC |
| SEQ ID NO 55 | 5′-Bile acid-CgTctaccaagctccacgTC |
| SEQ ID NO 56 | 5′-Bile acid-CgTctactggatcccacgTC |
| SEQ ID NO 57 | CgTctacccaagtccacgTC |
| SEQ ID NO 58 | CgTctacttggacccacgTC |
| SEQ ID NO 59 | CgTctacggcttaccacgTC |
| SEQ ID NO 60 | 5′-Tocopherol-CgTctacccgcggccacgTC |
| SEQ ID NO 61 | 5′-Tocopherol-CgTctacttataaccacgTC |

3 days after injection, the tissue samples were homogenized in RIPA buffer (Pierce) using Tissue Lyzer (Qiagen). Tissue were homogenized in a volume of RIPA buffer according to their weight in a ratio of 10 mg tissue/450 μL Ripa buffer. The homogenized liver, kidney and lung tissues were then diluted 100× in distilled water, whereas other tissues were diluted 10× in distilled water. DNase inactivation was done by incubating the samples at 75° C. for 40 min followed by 4° C. for 15 min. In parallel, two samples for reference normalization consisted of 10 μL of a solution containing 5 pM (each) oligonucleotide library and 490 μL RIPA buffer (final 100 nM each oligonucleotide).

4 μL of the RIPA solution was used in the first ligation reaction containing 2 μL Capture probe 1 (1 nM), 2 μL T4 ligase buffer, 6 μL PEG, 0.25 μL T4 Ligase (ThermoFisher Scientific) and 5.75 μL distilled water. Each tissue sample were ligated to a specific capture probe 1 with a specific index sequence for later identification of each individual sample see table below.

TABLE 20
Library of 32 Capture probe 1. Index
for identification is shown in bold

| Capture probe 1 | Sequence |
|---|---|
| BCS_CapP1_v6_I1 | /5Phos/CCGCAAGTGGCGTGATNNNNAGATCGGAAGAGCG |
| | TCGTGTAGTCCGCATGTCGCGTGATAGGGATATCTTGCGG |
| | GACGTG/3ddC/ (SEQ ID NO 62) |
| BCS_CapP1_v6_I2 | /5Phos/CCGCAAGTGGACATCGNNNNAGATCGGAAGAGCG |
| | TCGTGTAGTCCGCATGTCGCGTGATAGGGATATCTTGCGG |
| | GACGTG/3ddC/ (SEQ ID NO 63) |
| BCS_CapP1_v6_I3 | /5Phos/CCGCAAGTGGGCCTAANNNNAGATCGGAAGAGCG |
| | TCGTGTAGTCCGCATGTCGCGTGATAGGGATATCTTGCGG |
| | GACGTG/3ddC/ (SEQ ID NO 64) |
| BCS_CapP1_v6_I4 | /5Phos/CCGCAAGTGGTGGTCANNNNAGATCGGAAGAGCG |
| | TCGTGTAGTCCGCATGTCGCGTGATAGGGATATCTTGCGG |
| | GACGTG/3ddC/ (SEQ ID NO 65) |
| BCS_CapP1_v6_I5 | /5Phos/CCGCAAGTGGCACTGTNNNNAGATCGGAAGAGCGT |
| | CGTGTAGTCCGCATGTCGCGTGATAGGGATATCTTGCGGG |
| | ACGTG/3ddC/ (SEQ ID NO 66) |
| BCS_CapP1_v6_16 | /5Phos/CCGCAAGTGGATTGGCNNNNAGATCGGAAGAGCG |
| | TCGTGTAGTCCGCATGTCGCGTGATAGGGATATCTTGCGG |
| | GACGTG/3ddC/ (SEQ ID NO 67) |
| BCS_CapP1_v6_I7 | /5Phos/CCGCAAGTGGGATCTGNNNNAGATCGGAAGAGCG |
| | TCGTGTAGTCCGCATGTCGCGTGATAGGGATATCTTGCGG |
| | GACGTG/3ddC/ (SEQ ID NO 68) |
| BCS_CapP1_v6_18 | /5Phos/CCGCAAGTGGTCAAGTNNNNAGATCGGAAGAGCGT |
| | CGTGTAGTCCGCATGTCGCGTGATAGGGATATCTTGCGGG |
| | ACGTG/3ddC/ (SEQ ID NO 69) |
| BCS_CapP1_v6_I9 | /5Phos/CCGCAAGTGGAGCGCTNNNNAGATCGGAAGAGCG |
| | TCGTGTAGTCCGCATGTCGCGTGATAGGGATATCTTGCGG |
| | GACGTG/3ddC/ (SEQ ID NO 70) |
| BCS_CapP1_v6_110 | /5Phos/CCGCAAGTGGGATATCNNNNAGATCGGAAGAGCGT |
| | CGTGTAGTCCGCATGTCGCGTGATAGGGATATCTTGCGGG |
| | ACGTG/3ddC/ (SEQ ID NO 71) |
| BCS_CapP1_v6_111 | /5Phos/CCGCAAGTGGCGCAGANNNNAGATCGGAAGAGCG |
| | TCGTGTAGTCCGCATGTCGCGTGATAGGGATATCTTGCGG |
| | GACGTG/3ddC/ (SEQ ID NO 72) |
| BCS_CapP1_v6_I 12 | /5Phos/CCGCAAGTGGTATGAGNNNNAGATCGGAAGAGCG |
| | TCGTGTAGTCCGCATGTCGCGTGATAGGGATATCTTGCGG |
| | GACGTG/3ddC/ (SEQ ID NO 73) |
| BCS_CapP1_v6_I13 | /5Phos/CCGCAAGTGGAGGTGCNNNNAGATCGGAAGAGCG |
| | TCGTGTAGTCCGCATGTCGCGTGATAGGGATATCTTGCGG |
| | GACGTG/3ddC/ (SEQ ID NO 74) |
| BCS_CapP1_v6_I 14 | /5Phos/CCGCAAGTGGGAACATNNNNAGATCGGAAGAGCG |
| | TCGTGTAGTCCGCATGTCGCGTGATAGGGATATCTTGCGG |
| | GACGTG/3ddC/ (SEQ ID NO 75) |
| BCS_CapP1_v6_I 15 | /5Phos/CCGCAAGTGGACATAGNNNNAGATCGGAAGAGCG |
| | TCGTGTAGTCCGCATGTCGCGTGATAGGGATATCTTGCGG |
| | GACGTG/3ddC/ (SEQ ID NO 76) |
| BCS_CapP1_v6_I 16 | /5Phos/CCGCAAGTGGGTGCGANNNNAGATCGGAAGAGCG |
| | TCGTGTAGTCCGCATGTCGCGTGATAGGGATATCTTGCGG |
| | GACGTG/3ddC/ (SEQ ID NO 77) |
| BCS_CapP1_v6_I17 | /5Phos/CCGCAAGTGGCCAACANNNNAGATCGGAAGAGCG |
| | TCGTGTAGTCCGCATGTCGCGTGATAGGGATATCTTGCGG |
| | GACGTG/3ddC/ (SEQ ID NO 78) |
| BCS_CapP1_v6_I 18 | /5Phos/CCGCAAGTGGTTGGTGNNNNAGATCGGAAGAGCGT |
| | CGTGTAGTCCGCATGTCGCGTGATAGGGATATCTTGCGGG |
| | ACGTG/3ddC/ (SEQ ID NO 79) |
| BCS_CapP1_v6_I19 | /5Phos/CCGCAAGTGGCGCGGTNNNNAGATCGGAAGAGCG |
| | TCGTGTAGTCCGCATGTCGCGTGATAGGGATATCTTGCGG |
| | GACGTG/3ddC/ (SEQ ID NO 80) |
| BCS_CapP1_v6_120 | /5Phos/CCGCAAGTGGTATAACNNNNAGATCGGAAGAGCGT |
| | CGTGTAGTCCGCATGTCGCGTGATAGGGATATCTTGCGGG |
| | ACGTG/3ddC/ (SEQ ID NO 81) |
| BCS_CapP1_v6_I21 | /5Phos/CCGCAAGTGGAAGGATNNNNAGATCGGAAGAGCG |
| | TCGTGTAGTCCGCATGTCGCGTGATAGGGATATCTTGCGG |
| | GACGTG/3ddC/ (SEQ ID NO 82) |
| BCS_CapP1_v6_122 | /5Phos/CCGCAAGTGGGCAAGCNNNNAGATCGGAAGAGCG |
| | TCGTGTAGTCCGCATGTCGCGTGATAGGGATATCTTGCGG |
| | GACGTG/3ddC/ (SEQ ID NO 83) |
| BCS_CapP1_v6_123 | /5Phos/CCGCAAGTGGTCGTGANNNNAGATCGGAAGAGCG |
| | TCGTGTAGTCCGCATGTCGCGTGATAGGGATATCTTGCGG |
| | GACGTG/3ddC/ (SEQ ID NO 84) |
| BCS_CapP1_v6_124 | /5Phos/CCGCAAGTGGCTACAGNNNNAGATCGGAAGAGCG |
| | TCGTGTAGTCCGCATGTCGCGTGATAGGGATATCTTGCGG |
| | GACGTG/3ddC/ (SEQ ID NO 85) |
| BCS_CapP1_v6_125 | /5Phos/CCGCAAGTGGATATTCNNNNAGATCGGAAGAGCGT |
| | CGTGTAGTCCGCATGTCGCGTGATAGGGATATCTTGCGGG |
| | ACGTG/3ddC/ (SEQ ID NO 86) |
| BCS_CapP1_v6_126 | /5Phos/CCGCAAGTGGGCGCCTNNNNAGATCGGAAGAGCG |
| | TCGTGTAGTCCGCATGTCGCGTGATAGGGATATCTTGCGG |
| | GACGTG/3ddC/ (SEQ ID NO 87) |
| BCS_CapP1_v6_127 | /5Phos/CCGCAAGTGGACTCTANNNNAGATCGGAAGAGCGT |
| | CGTGTAGTCCGCATGTCGCGTGATAGGGATATCTTGCGGG |
| | ACGTG/3ddC/ (SEQ ID NO 88) |
| BCS_CapP1_v6_128 | /5Phos/CCGCAAGTGGGTCTCGNNNNAGATCGGAAGAGCG |
| | TCGTGTAGTCCGCATGTCGCGTGATAGGGATATCTTGCGG |
| | GACGTG/3ddC/ (SEQ ID NO 89) |
| BCS_CapP1_v6_129 | /5Phos/CCGCAAGTGGAAGACGNNNNAGATCGGAAGAGCG |
| | TCGTGTAGTCCGCATGTCGCGTGATAGGGATATCTTGCGG |
| | GACGTG/3ddC/ (SEQ ID NO 90) |
| BCS_CapP1_v6_130 | /5Phos/CCGCAAGTGGCGAGTANNNNAGATCGGAAGAGCG |
| | TCGTGTAGTCCGCATGTCGCGTGATAGGGATATCTTGCGG |
| | GACGTG/3ddC/ (SEQ ID NO 91) |
| BCS_CapP1_v6_I31 | /5Phos/CCGCAAGTGGAACCGCNNNNAGATCGGAAGAGCG |
| | TCGTGTAGTCCGCATGTCGCGTGATAGGGATATCTTGCGG |
| | GACGTG/3ddC/ (SEQ ID NO 92) |
| BCS_CapP1_v6_132 | /5Phos/CCGCAAGTGCGGTTATNNNNAGATCGGAAGAGCGT |
| | CGTGTAGTCCGCATGTCGCGTGATAGGGATATCTTGCGGG |
| | ACGTG/3ddC/ (SEQ ID NO 93) |

After incubation for 1 h at 16° C. and inactivation for 15 min at 75° C., 2p1 of the ligation reaction was used for first Strand synthesis in a reaction containing 2 μL of the ligation reaction, 4 μL 5× Volcano buffer (MyPols), 0.5 μL of first strand primer 100 nM, 0.5 μL 10 mM dNTP, 0.4 μL Volcano PG (MyPols), and 12.6 μL distilled water. PCR conditions were 95° C. for 2 min, followed by 15 cycles of 95° C. for 30 s and 60° C. for 30 min, and finally 72° C. for 5 min.

| First Strand primer | Sequence |
|---|---|
| RT_Primer | 5CCCTATGACGCGACATGCGGA3 |
| SeqCapP1_V6 | (SEQ ID NO 40) |

All the first strand PCRs product were pooled and 50 μL of the pooled first Strand PCR products were purified using the Monarch PCR and DNA clean up kit (New England Biolabs) according to manufactorer's instruction and eluated in 10 μL elution buffer. 4 μL of the eluted first strand product was mixed with 4 μL Capture probe 2 (100 nM) and heated to 60° C. for 5 min followed by a slow (0.1° C./s) decline in temperature to 4° C. Then 12 μL consisting of 2 μL T4 ligase buffer, 6 μL PEG, 0.5 μL T4 Ligase (ThermoFisher Scientific) and 3.5 μL distilled water was added.

| Capture probe 2 | Sequence |
|---|---|
| LNA_BC_seq_CapP2_v1 | /5Phos/CCG CAA GGA GAT CGG AAG |
| | AGC ACA CGT CTG AAC TCC ATA TGC |
| | TTG CGG CGT CTA C/3AmMO/ |
| | (SEQ ID NO 94) |

After incubation for 1 h at 4° C. and inactivation for 15 min at 75° C., 2 μL of the ligation reaction was applied to the PCR reaction containing 4 μL 5×HF buffer, 0.4 μL 10 mM dNTP, 1 μL 10 pM Forward Seq primer (PE1) and 1 μL 10 pM Reverse Seq primer (PE2V3), 0.2 μL Phusion DNA polymerase (New England Biolabs) and 11.4 μL distilled water. PCR conditions were 98° C. for 30 s, followed by 20 cycles of 98° C. for 15s, 60° C. for 20s, and 72° C. for 20 s, and finally 72° C. for 5 min.

| PCR | |
|---|---|
| Primers | Sequence |
| PE1 | 5AATGATACGGCGACCACCGAGATCTACACTCTTTCCCT |
| | ACACGACGCTCTTCCGATCT3 (SEQ ID NO 42) |
| PE2V3 | CAA GCA GAA GAC GGC ATA CGA GAT CGG TCT |
| | CGG GAG TTC AGA CGT GTG CTC TTC CGA TCT |
| | (SEQ ID NO 95) |

PCR product was purified using Qiaquick PCR purification kit (Qiagen) according to manufactorer's instructions and eluated in 30 μL distilled water. The PCR product was sequenced according to the protocol from Illumina MiniSeq System (Illumina). By using the software CLC Genomics Workbench, 11.0.1. The number of reads for the different Barcodes were identified and relative number of barcodes for each capture index (each tissue sample) were calculated. The relative number for each barcode were normalized (%) to the relative number of barcodes in the Index reference library 1 from the test tube reactions.

TABLE 21
Tissue samples number of reads. 3 days after 1 v injection in two C57BL/6J mice.

| | Index 1 | Index 2 | Index 9 | Index 24 | Index 3 | Index 18 | Index 6 | Index 21 |
|---|---|---|---|---|---|---|---|---|
| | ref_lib | ref_lib | Adipose_1 | Adipose_2 | Cortex_1 | Cortex_2 | Eye_1 | Eye_2 |
| SEQ | 52410 | 20126 | 2322 | 1070 | 26 | 10 | 142 | 188 |
| ID #47 | | | | | | | | |
| SEQ | 145798 | 67458 | 4588 | 2070 | 2 | 20 | 116 | 330 |
| ID #48 | | | | | | | | |
| SEQ | 196366 | 84194 | 1558 | 932 | 78 | 92 | 106 | 274 |
| ID #49 | | | | | | | | |
| SEQ | 47270 | 19462 | 450 | 212 | 8 | 36 | 48 | 56 |
| ID #50 | | | | | | | | |
| SEQ | 65174 | 41742 | 2046 | 756 | 12 | 28 | 236 | 332 |
| ID #51 | | | | | | | | |
| SEQ | 87316 | 49796 | 2520 | 912 | 30 | 22 | 198 | 320 |
| ID #52 | | | | | | | | |
| SEQ | 79232 | 49208 | 2898 | 896 | 26 | 24 | 204 | 372 |
| ID #53 | | | | | | | | |
| SEQ | 48344 | 26708 | 916 | 516 | 22 | 12 | 106 | 234 |
| ID #54 | | | | | | | | |
| SEQ | 34434 | 19772 | 776 | 314 | 0 | 16 | 70 | 120 |
| ID #55 | | | | | | | | |
| SEQ | 41238 | 27040 | 926 | 444 | 2 | 4 | 96 | 200 |
| ID #56 | | | | | | | | |
| SEQ | 202906 | 106560 | 4590 | 2266 | 6 | 8 | 362 | 686 |
| ID #57 | | | | | | | | |
| SEQ | 222504 | 96394 | 4272 | 2648 | 28 | 30 | 500 | 590 |
| ID #58 | | | | | | | | |
| SEQ | 339218 | 156754 | 8276 | 4206 | 32 | 46 | 622 | 1136 |
| ID #59 | | | | | | | | |
| SEQ | 85968 | 33400 | 382 | 206 | 36 | 34 | 60 | 250 |
| ID #60 | | | | | | | | |
| SEQ | 221606 | 101102 | 1676 | 804 | 36 | 32 | 262 | 524 |
| ID #61 | | | | | | | | |
| Total | 1869784 | 899716 | 38196 | 18252 | 344 | 414 | 3128 | 5612 |
| reads | | | | | | | | |
| | Index 10 | Index 25 | Index 7 | Index 22 | Index 13 | Index 28 | Index 17 | Index 32 |
| | Femur_1 | Femur_2 | Heart_1 | Heart_2 | Illium_1 | Illium_2 | Kidney_1 | Kidney_2 |
| SEQ | 3190 | 3196 | 2354 | 1570 | 1210 | 1128 | 1512 | 530 |
| ID #47 | | | | | | | | |
| SEQ | 5338 | 3838 | 2186 | 2258 | 1326 | 1290 | 1988 | 1104 |
| ID #48 | | | | | | | | |
| SEQ | 2028 | 2364 | 2430 | 2946 | 1674 | 1402 | 1406 | 586 |
| ID #49 | | | | | | | | |
| SEQ | 874 | 872 | 750 | 1032 | 512 | 348 | 384 | 158 |
| ID #50 | | | | | | | | |
| SEQ | 4188 | 4272 | 1526 | 1272 | 6684 | 5456 | 3762 | 3614 |
| ID #51 | | | | | | | | |
| SEQ | 4276 | 3272 | 1340 | 1206 | 4954 | 4638 | 3888 | 3336 |
| ID #52 | | | | | | | | |
| SEQ | 4316 | 4424 | 1656 | 1128 | 5784 | 6018 | 3792 | 3408 |
| ID #53 | | | | | | | | |
| SEQ | 2282 | 2040 | 1038 | 910 | 3550 | 3352 | 2176 | 1740 |
| ID #54 | | | | | | | | |
| SEQ | 1230 | 1414 | 414 | 500 | 2230 | 1998 | 1572 | 1200 |
| ID #55 | | | | | | | | |
| SEQ | 2002 | 2084 | 874 | 660 | 3644 | 3816 | 2302 | 1708 |
| ID #56 | | | | | | | | |
| SEQ | 12980 | 14522 | 3860 | 1964 | 12306 | 12064 | 16504 | 13714 |
| ID #57 | | | | | | | | |
| SEQ | 14514 | 16224 | 4134 | 2614 | 10698 | 11986 | 24720 | 16690 |
| ID #58 | | | | | | | | |
| SEQ | 20832 | 23202 | 7126 | 4332 | 21788 | 23826 | 28592 | 26306 |
| ID #59 | | | | | | | | |
| SEQ | 1058 | 1570 | 1538 | 1522 | 1042 | 744 | 704 | 306 |
| ID #60 | | | | | | | | |
| SEQ | 2860 | 2562 | 3096 | 3130 | 2356 | 2124 | 2518 | 910 |
| ID #61 | | | | | | | | |
| Total | 81968 | 85856 | 34322 | 27044 | 79758 | 80190 | 95820 | 75310 |
| reads | | | | | | | | |
| | | | | | Index 8 | Index 23 | | |
| | Index 16 | Index 31 | Index 15 | Index 30 | Lymph | Lymph | Index 12 | Index 27 |
| | Liver_1 | Liver_2 | Lung_1 | Lung_2 | node_1 | node_2 | Pancreas_1 | Pancreas_2 |
| SEQ | 1138 | 3512 | 78 | 208 | 2104 | 8244 | 906 | 358 |
| ID #47 | | | | | | | | |
| SEQ | 2410 | 9480 | 70 | 278 | 2596 | 8358 | 1786 | 1402 |
| ID #48 | | | | | | | | |
| SEQ | 5428 | 16462 | 172 | 414 | 2812 | 8844 | 1866 | 1536 |
| ID #49 | | | | | | | | |
| SEQ | 1656 | 4650 | 44 | 108 | 940 | 3120 | 512 | 248 |
| ID #50 | | | | | | | | |
| SEQ | 1642 | 5590 | 36 | 80 | 10746 | 25208 | 17124 | 16138 |
| ID #51 | | | | | | | | |
| SEQ | 1610 | 4710 | 22 | 120 | 7050 | 18978 | 11780 | 11358 |
| ID #52 | | | | | | | | |
| SEQ | 1546 | 4184 | 62 | 174 | 11256 | 28164 | 6918 | 3794 |
| ID #53 | | | | | | | | |
| SEQ | 896 | 2684 | 16 | 72 | 8586 | 20950 | 6220 | 1912 |
| ID #54 | | | | | | | | |
| SEQ | 816 | 2014 | 22 | 20 | 4904 | 11564 | 4198 | 2530 |
| ID #55 | | | | | | | | |
| SEQ | 922 | 2672 | 28 | 54 | 6216 | 16346 | 3692 | 2002 |
| ID #56 | | | | | | | | |
| SEQ | 2274 | 4490 | 126 | 284 | 26474 | 52540 | 7772 | 3192 |
| ID #57 | | | | | | | | |
| SEQ | 2140 | 4136 | 150 | 288 | 24270 | 42040 | 6412 | 2590 |
| ID #58 | | | | | | | | |
| SEQ | 3672 | 9408 | 226 | 342 | 36940 | 74732 | 14154 | 7608 |
| ID #59 | | | | | | | | |
| SEQ | 3414 | 7958 | 38 | 128 | 1790 | 5876 | 200 | 218 |
| ID #60 | | | | | | | | |
| SEQ | 7022 | 20470 | 72 | 282 | 4476 | 14072 | 702 | 362 |
| ID #61 | | | | | | | | |
| Total | 36586 | 102420 | 1162 | 2852 | 151160 | 339036 | 84242 | 55248 |
| reads | | | | | | | | |
| | | | Index 11 | Index 26 | | | | |
| | Index 5 | Index 20 | Spinal | Spinal | Index 4 | Index 19 | Index 14 | Index 29 |
| | Serum_1 | Serum_2 | cord_1 | cord_2 | Spleen_1 | Spleen_2 | Stomach_1 | Stomach_2 |
| SEQ | 14 | 8 | 6 | 24 | 1868 | 2192 | 206 | 528 |
| ID #47 | | | | | | | | |
| SEQ | 34 | 86 | 4 | 14 | 2956 | 4970 | 180 | 470 |
| ID #48 | | | | | | | | |
| SEQ | 40 | 108 | 54 | 112 | 9722 | 11224 | 104 | 460 |
| ID #49 | | | | | | | | |
| SEQ | 14 | 68 | 4 | 36 | 1946 | 2396 | 54 | 148 |
| ID #50 | | | | | | | | |
| SEQ | 20 | 16 | 2 | 30 | 3676 | 5620 | 1174 | 168 |
| ID #51 | | | | | | | | |
| SEQ | 26 | 54 | 12 | 22 | 2702 | 3508 | 1270 | 1996 |
| ID #52 | | | | | | | | |
| SEQ | 16 | 30 | 10 | 36 | 2530 | 2278 | 1068 | 2006 |
| ID #53 | | | | | | | | |
| SEQ | 4 | 6 | 0 | 10 | 1972 | 2372 | 686 | 1524 |
| ID #54 | | | | | | | | |
| SEQ | 4 | 12 | 4 | 24 | 1354 | 1092 | 610 | 1240 |
| ID #55 | | | | | | | | |
| SEQ | 12 | 2 | 8 | 22 | 1894 | 1186 | 562 | 304 |
| ID #56 | | | | | | | | |
| SEQ | 52 | 46 | 16 | 48 | 9260 | 8196 | 2260 | 4676 |
| ID #57 | | | | | | | | |
| SEQ | 54 | 28 | 18 | 40 | 8932 | 6500 | 2428 | 4096 |
| ID #58 | | | | | | | | |
| SEQ | 68 | 54 | 46 | 48 | 14664 | 22976 | 3914 | 4080 |
| ID #59 | | | | | | | | |
| SEQ | 32 | 36 | 50 | 30 | 4810 | 6180 | 150 | 64 |
| ID #60 | | | | | | | | |
| SEQ | 92 | 84 | 18 | 62 | 7410 | 8644 | 208 | 554 |
| ID #61 | | | | | | | | |
| Total | 482 | 638 | 252 | 558 | 75696 | 89334 | 14874 | 22314 |
| reads | | | | | | | | |

TABLE 22
Relative abundance of reads for barcode oligos in each tissue sample. normalized to relative
abundance of the reads for reference library 1 (%). Relative abundance of reads shows that
tocopherol conjugation (compound SEQ ID 58 and SED ID 59) and cholesterol conjugation
(SEQ ID 47 and SEQ ID 48) have increased liver distribution to the liver and reduced
Kidney distribution compared to naked oligos SEQ ID 55, 56 and 57 and other conjugations.
Bile amine conjugation SEQ ID 49 and SEQ ID 50) show increased content in Pancreas.

| | Index 1 | Index 2 | Index9 | Index 24 | Index 3 | Index 18 | Index 6 | Index 21 |
|---|---|---|---|---|---|---|---|---|
| | ref_lib | ref_lib | Adipose_1 | Adipose_2 | Cortex_1 | Cortex_2 | Eye_1 | Eye_2 |
| SEQ | 100 | 79.8 | 216.9 | 209.1 | 269.6 | 86.2 | 162 | 119.5 |
| ID #47 | | | | | | | | |
| SEQ | 100 | 96.2 | 154 | 145.4 | 7.5 | 62 | 47.6 | 75.4 |
| ID #48 | | | | | | | | |
| SEQ | 100 | 89.1 | 38.8 | 48.6 | 215.9 | 211.6 | 32.3 | 46.5 |
| ID #49 | | | | | | | | |
| SEQ | 100 | 85.6 | 46.6 | 45.9 | 92 | 344 | 60.7 | 39.5 |
| ID #50 | | | | | | | | |
| SEQ | 100 | 133.1 | 153.7 | 118.8 | 100.1 | 194 | 216.5 | 169.7 |
| ID #51 | | | | | | | | |
| SEQ | 100 | 118.5 | 141.3 | 107 | 186.7 | 113.8 | 135.5 | 122.1 |
| ID #52 | | | | | | | | |
| SEQ | 100 | 129.1 | 179 | 115.8 | 178.4 | 136.8 | 153.9 | 156.4 |
| ID #53 | | | | | | | | |
| SEQ | 100 | 114.8 | 92.8 | 109.3 | 247.4 | 112.1 | 131.1 | 161.3 |
| ID #54 | | | | | | | | |
| SEQ | 100 | 119.3 | 110.3 | 93.4 | 0 | 209.9 | 121.5 | 116.1 |
| ID #55 | | | | | | | | |
| SEQ | 100 | 136.3 | 109.9 | 110.3 | 26.4 | 43.8 | 139.2 | 161.6 |
| ID #56 | | | | | | | | |
| SEQ | 100 | 109.1 | 110.7 | 114.4 | 16.1 | 17.8 | 106.6 | 112.6 |
| ID #57 | | | | | | | | |
| SEQ | 100 | 90 | 94 | 121.9 | 68.4 | 60.9 | 134.3 | 88.3 |
| ID #58 | | | | | | | | |
| SEQ | 100 | 96 | 119.4 | 127 | 51.3 | 61.2 | 109.6 | 111.6 |
| ID #59 | | | | | | | | |
| SEQ | 100 | 80.7 | 21.8 | 24.5 | 227.6 | 178.6 | 41.7 | 96.9 |
| ID #60 | | | | | | | | |
| SEQ | 100 | 94.8 | 37 | 37.2 | 88.3 | 65.2 | 70.7 | 78.8 |
| ID #61 | | | | | | | | |
| | Index 10 | Index 25 | Index7 | Index 22 | Index 13 | Index 28 | Index 17 | Index 32 |
| | Femur_1 | Femur_2 | Heart_1 | Heart_2 | Illium_1 | Illium_2 | Kidney_1 | Kidney_2 |
| SEQ | 138.8 | 132.8 | 244.7 | 207.1 | 54.1 | 50.2 | 56.3 | 25.1 |
| ID #47 | | | | | | | | |
| SEQ | 83.5 | 57.3 | 81.7 | 107.1 | 21.3 | 20.6 | 26.6 | 18.8 |
| ID #48 | | | | | | | | |
| SEQ | 23.6 | 26.2 | 67.4 | 103.7 | 20 | 16.6 | 14 | 7.4 |
| ID #49 | | | | | | | | |
| SEQ | 42.2 | 40.2 | 86.4 | 150.9 | 25.4 | 17.2 | 15.9 | 8.3 |
| I D #50 | | | | | | | | |
| SEQ | 146.6 | 142.8 | 127.6 | 134.9 | 240.4 | 195.2 | 112.6 | 137.7 |
| ID #51 | | | | | | | | |
| SEQ | 111.7 | 81.6 | 83.6 | 95.5 | 133 | 123.9 | 86.9 | 94.9 |
| ID #52 | | | | | | | | |
| SEQ | 124.3 | 121.6 | 113.9 | 98.4 | 171.1 | 177.1 | 93.4 | 106.8 |
| ID #53 | | | | | | | | |
| SEQ | 107.7 | 91.9 | 117 | 130.1 | 172.1 | 161.7 | 87.8 | 89.4 |
| ID #54 | | | | | | | | |
| SEQ | 81.5 | 89.4 | 65.5 | 100.4 | 151.8 | 135.3 | 89.1 | 86.5 |
| ID #55 | | | | | | | | |
| SEQ | 110.7 | 110.1 | 115.5 | 110.7 | 207.2 | 215.8 | 108.9 | 102.8 |
| ID #56 | | | | | | | | |
| SEQ | 145.9 | 155.9 | 103.6 | 66.9 | 142.2 | 138.6 | 158.7 | 167.8 |
| ID #57 | | | | | | | | |
| SEQ | 148.8 | 158.8 | 101.2 | 81.2 | 112.7 | 125.6 | 216.8 | 186.2 |
| ID #58 | | | | | | | | |
| SEQ | 140.1 | 149 | 114.4 | 88.3 | 150.6 | 163.8 | 164.5 | 192.5 |
| ID #59 | | | | | | | | |
| SEQ | 28.1 | 39.8 | 97.5 | 122.4 | 28.4 | 20.2 | 16 | 8.8 |
| ID #60 | | | | | | | | |
| SEQ | 29.4 | 25.2 | 76.1 | 97.7 | 24.9 | 22.3 | 22.2 | 10.2 |
| ID #61 | | | | | | | | |
| | | | | | Index 8 | Index 23 | | |
| | Index 16 | Index 31 | Index 15 | Index 30 | Lymph | Lymph | Index 12 | Index 27 |
| | Liver_1 | Liver_2 | Lung_1 | Lung_2 | node_1 | node_2 | Pancreas_1 | Pancreas_2 |
| SEQ | 111.0 | 122.3 | 239.5 | 260.2 | 49.7 | 86.7 | 38.4 | 23.1 |
| ID #47 | | | | | | | | |
| SEQ | 84.5 | 118.7 | 77.3 | 125.0 | 22.0 | 31.6 | 27.2 | 32.5 |
| ID #48 | | | | | | | | |
| SEQ | 141.3 | 153.0 | 140.9 | 138.2 | 17.7 | 24.8 | 21.1 | 26.5 |
| ID #49 | | | | | | | | |
| SEQ | 179.0 | 179.6 | 149.8 | 149.8 | 24.6 | 36.4 | 24.0 | 17.8 |
| ID #50 | | | | | | | | |
| SEQ | 128.8 | 156.6 | 88.9 | 80.5 | 204.0 | 213.3 | 583.2 | 838.0 |
| ID #51 | | | | | | | | |
| SEQ | 94.2 | 98.5 | 40.5 | 90.1 | 99.9 | 119.9 | 299.4 | 440.2 |
| ID #52 | | | | | | | | |
| SEQ | 99.7 | 96.4 | 125.9 | 144.0 | 175.7 | 196.0 | 193.8 | 162.1 |
| ID #53 | | | | | | | | |
| SEQ | 94.7 | 101.4 | 53.3 | 97.6 | 219.7 | 239.0 | 285.6 | 133.9 |
| ID #54 | | | | | | | | |
| SEQ | 121.1 | 106.8 | 102.8 | 38.1 | 176.2 | 185.2 | 270.6 | 248.7 |
| ID #55 | | | | | | | | |
| SEQ | 114.3 | 118.3 | 109.3 | 85.8 | 186.5 | 218.6 | 198.7 | 164.3 |
| ID #56 | | | | | | | | |
| SEQ | 57.3 | 40.4 | 99.9 | 91.8 | 161.4 | 142.8 | 85.0 | 53.2 |
| ID #57 | | | | | | | | |
| SEQ | 49.2 | 33.9 | 108.5 | 84.9 | 134.9 | 104.2 | 64.0 | 39.4 |
| ID #58 | | | | | | | | |
| SEQ | 55.3 | 50.6 | 107.2 | 66.1 | 134.7 | 121.5 | 92.6 | 75.9 |
| ID #59 | | | | | | | | |
| SEQ | 203.0 | 169.0 | 71.1 | 97.6 | 25.8 | 37.7 | 5.2 | 8.6 |
| ID #60 | | | | | | | | |
| SEQ | 161.9 | 168.6 | 52.3 | 83.4 | 25.0 | 35.0 | 7.0 | 5.5 |
| ID#61 | | | | | | | | |
| | | | Index 11 | Index 26 | | | | |
| | Index 5 | Index 20 | Spinal | Spinal | Index 4 | Index 19 | Index 14 | Index 29 |
| | Serum_1 | Serum_2 | cord_1 | cord_2 | Spleen_1 | Spleen_2 | Stomach_1 | Stomach_2 |
| SEQ | 103.6 | 44.7 | 84.9 | 153.4 | 88.0 | 87.5 | 49.4 | 84.4 |
| ID #47 | | | | | | | | |
| SEQ | 90.5 | 172.9 | 20.4 | 32.2 | 50.1 | 71.3 | 15.5 | 27.0 |
| ID# 48 | | | | | | | | |
| SEQ | 79.0 | 161.2 | 204.0 | 191.1 | 122.3 | 119.6 | 6.7 | 19.6 |
| ID #49 | | | | | | | | |
| SEQ | 114.9 | 421.6 | 62.8 | 255.2 | 101.7 | 106.1 | 14.4 | 26.2 |
| ID #50 | | | | | | | | |
| SEQ | 119.0 | 71.9 | 22.8 | 154.2 | 139.3 | 180.5 | 226.4 | 21.6 |
| ID #51 | | | | | | | | |
| SEQ | 115.5 | 181.2 | 102.0 | 84.4 | 76.4 | 84.1 | 182.8 | 191.5 |
| ID #52 | | | | | | | | |
| SEQ | 78.3 | 111.0 | 93.6 | 152.3 | 78.9 | 60.2 | 169.4 | 212.2 |
| ID #53 | | | | | | | | |
| SEQ | 32.1 | 36.4 | 0.0 | 69.3 | 100.8 | 102.7 | 178.4 | 264.2 |
| ID #54 | | | | | | | | |
| SEQ | 45.1 | 102.1 | 86.2 | 233.6 | 97.1 | 66.4 | 222.7 | 301.8 |
| ID #55 | | | | | | | | |
| SEQ | 112.9 | 14.2 | 143.9 | 178.8 | 113.4 | 60.2 | 171.3 | 61.8 |
| ID #56 | | | | | | | | |
| SEQ | 99.4 | 66.4 | 58.5 | 79.3 | 112.7 | 84.5 | 140.0 | 193.1 |
| ID #57 | | | | | | | | |
| SEQ | 94.1 | 36.9 | 60.0 | 60.2 | 99.2 | 61.1 | 137.2 | 154.3 |
| ID #58 | | | | | | | | |
| SEQ | 77.8 | 46.7 | 100.6 | 47.4 | 106.8 | 141.8 | 145.0 | 100.8 |
| ID #59 | | | | | | | | |
| SEQ | 144.4 | 122.7 | 431.5 | 116.9 | 138.2 | 150.5 | 21.9 | 6.2 |
| ID #60 | | | | | | | | |
| SEQ | 161.0 | 111.1 | 60.3 | 93.7 | 82.6 | 81.6 | 11.8 | 20.9 |
| ID #61 | | | | | | | | |

1. A method for sequencing the nucleobase sequence of a modified oligonucleotide, such as an 2′ sugar modified oligonucleotide, such as a LNA or 2′-O-methoxyethyl modified phosphorothioate oligonucleotide, said method comprising:
Step I: Perform polymerase mediated 5′-3′ first strand synthesis from the 2′ sugar modified oligonucleotide to produce a nucleic acid sequence comprising the complement of the 2′ sugar modified oligonucleotide;
Step II: Perform primer based sequencing of the first strand synthesis product obtained in step I.

2. The method according to claim 1, wherein after step I and prior to step II the first strand product of step I is PCR amplified.

3. The method according to claim 1, wherein step II comprises the clonal amplification of the first strand synthesis products or PCR amplification products, prior to the primer based sequencing.

4. The method according to claim 1, wherein the method is for the parallel sequencing of multiple modified oligonucleotide species within a population of modified oligonucleotides species present in a modified oligonucleotide sample.

5. The method according to claim 1, wherein prior to the polymerase mediated 5′-3′ first strand synthesis, a first primer which is complementary to a 3′ region of the modified oligonucleotide is hybridized to the modified oligonucleotide for initiation of the 5′-3′ first strand synthesis.

6. The method according to claim 1, wherein prior to the polymerase mediated 5′-3′ first strand synthesis, the modified oligonucleotide is ligated to a 3′ capture probe which is either a self-priming capture probe or comprises a first primer binding site.

7. The method according to claim 6, wherein prior to the first strand synthesis step a first primer is hybridized to the 3′ capture probe for initiation of the polymerase mediated first strand synthesis.

8. The method according to claim 6, wherein after ligation of the 3′ capture probe to the modified oligonucleotide, and prior to first strand synthesis the ligation product is purified e.g. via gel purification, or via enzymatic degradation of the un-ligated 3′ capture probe.

9. The method according to claim 6, wherein the PCR step is performed using a PCR primer pair, wherein one of the PCR primers is specific for the 3′ capture probe, and the second PCR primer is specific for the modified oligonucleotide, such as a 5′ region of the modified oligonucleotide.

10. The method according to claim 6, wherein the sequencing step II comprises the clonal amplification of the first strand product of step I using a clonal amplification primers, wherein one of the clonal amplification primers is specific for the 3′ capture probe, and the second clonal amplification primer is specific for the modified oligonucleotide such as a 5′ region of the modified oligonucleotide.

11. The method according to claim 6, wherein after the first strand synthesis step I, and first strand synthesis product purification if performed, an adapter probe is ligated at the 3′ end of the first strand synthesis product.

12. The method according to claim 11, wherein the PCR step is performed using a pair of PCR primers wherein one of the PCR primers is specific for the 3′ capture probe and the other PCR primer is specific for adapter probe.

13. The method according to claim 11, wherein the sequencing step II comprises the clonal amplification of the first strand synthesis product of step I using clonal amplification primers specific for the 3′ capture probe and adapter probe respectively.

14. The method according to claim 1, wherein after first strand synthesis, the first strand synthesis product is polyadenylated.

15. The method according to claim 14, wherein a second strand is synthesized from the first strand using a poly T primer.

16. The method according to claim 15, wherein the polyT primer further comprises a PCR primer binding site and/or a clonal amplification primer binding site.

17. The method according to claim 14, wherein the PCR step is performed using a primer which is specific for the 3′ capture probe and either the poly T primer or a PCR primer which is specific for the PCR primer binding site in the poly T primer.

18. The method according to claim 14, wherein the PCR step is performed using a primer which is specific for modified oligonucleotide, such as the 3′ region of the modified oligonucleotide, and either the poly T primer or a PCR primer which is specific for the PCR primer binding site in the poly T primer.

19. The method according to claim 14, wherein the sequencing step comprises clonal amplification wherein one of the clonal amplification primers is specific for the 3′ capture probe or the modified oligonucleotide (such as 3′region of the modified oligonucleotide), and the second clonal amplification primer is specific for the poly T primer.

20. The method according to claim 18, wherein the PCR primers further comprise clonal amplification primer binding sites, such as flow cell capture probe binding sites, wherein the sequencing step comprises clonal amplification using clonal amplification primers, such as flow cell binding primers, which are complementary to the clonal amplification primer binding sites.

21. The method according to claim 1, wherein prior to first strand synthesis wherein prior to the polymerase mediated 5′-3′ first strand synthesis, the modified oligonucleotide is ligated to a 5′ capture probe which comprises a primer binding site.

22. The method according to claim 21, wherein either the modified oligonucleotide comprises a 5′ phosphate group, or prior to the ligation of the 5′ capture probe the 5′ terminus of the modified oligonucleotide is phosphorylated.

23. The method according to claim 21, wherein the PCR step is performed using a first PCR primer specific for the modified oligonucleotide, such as the 3′ region of the modified oligonucleotide, and a second PCR primer which is specific for the 5′capture probe.

24. The method according to claim 21, wherein prior to first strand synthesis, a 3′ capture probe is ligated to the 3′ terminus of the modified oligonucleotide, either prior to or subsequent to or simultaneous to the ligation of the 5′ capture probe to the 5′ terminus of the modified oligonucleotide, wherein the 3′ capture probe is either a self-priming capture probe or comprises a first primer binding site.

25. The method according to claim 21, wherein prior to first strand synthesis the ligation product(s) is purified e.g. via gel purification, or via enzymatic degradation of the unligated 5′, and if used 3′ capture probes.

26. The method according to claim 24, wherein the first strand synthesis is initiated using a first primer hybridized to the 3′ capture probe or from the self-priming 3′ capture probe.

27. The method according to claim 24, wherein the PCR step is performed using a first PCR primer which is specific for the 3′ capture probe and a second PCR primer which is specific for the 5′ capture probe.

28. The method according to claim 23, wherein the PCR primers comprise clonal amplification primer binding sites (such as flow cell primer binding sites), and the sequencing step comprises a clonal amplification.

29. The method according to claim 24, wherein the second sequencing step comprises the clonal amplification of the first strand product of step I using a clonal amplification primers, wherein one of the clonal amplification primers is specific for the 3′ capture probe, and the second clonal amplification primer is specific for the modified oligonucleotide such as a 5′ region of the modified oligonucleotide.

30. The method according to claim 6, wherein the 3′ capture probe is ligated to the 3′ end of the modified oligonucleotide using T4 DNA ligase.

31. The method according to claim 21, wherein the 5′ capture probe is ligated to the 5′ end of the modified oligonucleotide using T4 RNA ligase or T4 RNA ligase II.

32. The method according to claim 1, wherein the sequencing step II comprises clonal amplification and the clonal amplification primers are bound to a solid support or are compartmentalized within an emulsion droplet.

33. The method according to claim 32, wherein the solid phase amplification is solid phase bridge amplification.

34. The method according to claim 1, wherein the primer based sequencing method is performed using sequencing by synthesis method.

35. The method according to claim 1, wherein the primer based sequencing method is a cyclic reversible termination method (CRT).

36. The method according to claim 1, wherein the sequencing is a parallel sequencing method, such as massively parallel sequencing.

37. The method according to claim 1, wherein the primer based sequencing is performed using clonal bridge amplification (e.g. Illumina sequencing-reversible dye terminator), or clonal emulsion PCR (Roche 454, GS FLX Titanium, Life Technologies SOLiD4, Life Technologies Ion Proton).

38. The method according to claim 1, wherein the first strand synthesis is performed in the presence of a polymerase and polyethylene glycol or propylene glycol.

39. The method according to claim 1, wherein the polymerase used for first strand synthesis is Taq polymerase or Volcano2G polymerase or PrimeScript reverse transcriptase.

40. The method according to claim 2, wherein the sequencing step comprises the clonal amplification of the PCR amplification product(s) using clonal amplification primers.

41. The method according to claim 1, wherein the 2′ sugar modified oligonucleotide is a 2′ sugar modified phosphorothioate oligonucleotide.

42. The method according to claim 1, wherein the modified oligonucleotide comprises at least two contiguous 2′ sugar modified nucleosides.

43. The method according to claim 1, wherein the modified oligonucleotide comprises at least one 2′-O-methoxyethyl RNA (MOE) nucleoside.

44. The method according to claim 1, wherein the modified oligonucleotide comprises at least two contiguous 2′-O-methoxyethyl RNA (MOE) nucleosides.

45. The method according to claim 1, wherein the modified oligonucleotide comprises at least one 2′-O-methoxyethyl RNA (MOE) nucleoside located at the 3′ of the modified oligonucleotide, such as at least two or at least three contiguous 2′-O-methoxyethyl RNA (MOE) nucleosides located at the 3′ end of the modified oligonucleotide.

46. The method according to claim 1, wherein the modified oligonucleotide comprises at least 1 LNA nucleoside.

47. The method according to claim 1, wherein the modified oligonucleotide comprises at least two contiguous LNA nucleotides or at least three contiguous LNA nucleotides.

48. The method according to claim 1, wherein the modified oligonucleotide comprises one or more LNA nucleotide(s) are located at the 3′ end.

49. The method according to claim 1, wherein the modified oligonucleotide is a LNA phosphorothioate oligonucleotide.

50. The method according to claim 1, wherein the modified oligonucleotide comprises both LNA nucleosides and DNA nucleosides, such as a LNA gapmer, or LNA mixmer.

51. The method according to claim 1, wherein the LNA oligonucleotide comprises at least one LNA-T nucleoside or at least one LNA-C nucleoside.

52. The method according to claim 1, wherein the modified oligonucleotide comprises one or more LNA nucleoside(s) and one or more 2′ substituted nucleoside, such as one or more 2′-O-methoxyethyl nucleosides.

53. The method according to claim 1, wherein the modified oligonucleotide is selected from the group consisting of; a 2′-O-methoxyethyl gapmer, a mixed wing gapmer, an alternating flank gapmer or a LNA gapmer.

54. The method according to claim 1, wherein the modified oligonucleotide is a mixmer or a totalmer.

55. The method according to claim 1, wherein the modified oligonucleotide comprise a conjugate group, such as a GalNAc conjugate.

56. The method according to claim 1, wherein said method is for determining the degree of purity or heterogeneity in the population of modified oligonucleotides, e.g. a single oligonucleotide synthesis batch or a pool of multiple oligonucleotide synthesis batches.

57. The method according to claim 1, wherein said method is for determining the sequence of the modified oligonucleotide, or the predominant sequences present in the population of modified oligonucleotides, e.g. a modified oligonucleotide synthesis batch or a pool of multiple oligonucleotide synthesis batches.

58. The method according to claim 1, wherein the modified oligonucleotide is a population of modified oligonucleotides, e.g. a population of modified oligonucelotides from the same oligonucleotide synthesis run or a pool of oligonucleotide synthesis runs.

59. The method according to claim 1, wherein the modified oligonucleotide is a population of modified oligonucleotides isolated from a cell, or a tissue or an organism.

60. The method according to claim 1, wherein the method is for identifying a modified oligonucleotide which is preferentially targeted to a target tissue or a target cell, wherein said method comprises of:
administering a mixture of modified oligonucleotides to an organism, such as a mammal, wherein each member of the mixture of modified oligonucleotides comprises a unique nucleobase sequence,
allow for the modified oligonucleotides to be distributed within the mammal and/or taken up by the cells;
isolate a population of modified oligonucleotides from one or more tissues or cells from the mammal, including a desired target tissue or desired target cell,
perform the method according to any one of the preceding claims, to
identify one or more modified oligonucleotide sequences which are enriched in the desired target tissue or cell of the mammal.

61. The method according to claim 1, wherein the method is for identifying a modified oligonucleotide which is preferentially taken up by a cell, such as a target cell, wherein said method comprises of:
administering a mixture of modified oligonucleotides to a cell, wherein each member of the mixture of modified oligonucleotides comprises a unique nucleobase sequence,
incubate the cells for a period of time;
isolate a population of modified oligonucleotides from the cell,
perform the method according to claim 1, to
identify one or more modified oligonucleotide sequences which are enriched in the cell.

62. The method according to claim 1, wherein the modified oligonucleotide comprises a population of modified oligonucleotides, wherein each member of the population of modified oligonucleotides comprises an aptameric region of nucleotides.

63. The method according to claim 1, wherein the modified oligonucleotide comprises a population of modified oligonucleotides, wherein each member of the population of modified oligonucleotides comprises a different conjugate group.

64. The method according to claim 1, wherein the 5′ capture probe is as defined in claim 1.

65. A 5′ capture probe oligonucleotide, for T4 RNA ligase or T4 RNA ligase II mediated ligation to the 5′ terminus of 5′ phosphorylated modified oligonucleotides, comprising 3′-5′:
a first region comprising a 3′terminal RNA nucleoside or a 3′ terminal region of RNA nucleotides with a 3′ terminal —OH group;
a second region of nucleotides which comprises the complement of a primer binding site, e.g. a PCR primer binding site (or a primer site for second strand synthesis);
a third region, comprising:
a sequence of nucleotides; or
a linker region which blocks polymerase read through; or
a 5′ terminal group other than 5′ phosphate;
a fourth region of nucleotides which is either:
covalently attached with the third region, or
in the event that the third region is a 5′ terminal group other than 5′-phosphase is discontinuous with the fourth region;
a fifth region of nucleotides which are complementary to a 5′ region of the modified oligonucleotide, or may optionally be a region of degenerate nucleotides;
a 5′ blocking group—i.e. a 5′ group other than 5′ phosphate.
wherein the second region and the fourth region comprise complementary regions of nucleotides which are capable of forming a duplex between the second a fourth regions.

66. The 5′ capture probe of claim 65, wherein the first region comprises 2-20 RNA nucleotides, such as 3-10 RNA nucleotides.

67. The 5′ capture probe of claim 65, wherein the second region comprises a sequencing primer binding site and/or a flow cell binding site.

68. The 5′ capture probe of claim 65, wherein the second region further comprises a molecular bar-code region.

69. The 5′ capture probe of claim 65, wherein the second region further comprises a sequencing reaction bar-code region.

70. The 5′ capture probe of claim 65, wherein the second region further comprises a sequencing primer binding region.

71. The 5′ capture probe according to claim 65, which comprises a linker region III which prevents polymerase read through during first strand synthesis.

72. The 5′capture probe according to claim 71, wherein the linker region III comprises a non-nucleotide linker, such as a C₆₋₃₂ polyethyleneglycol linker, such as a C18 polyethyleneglycol linker or an alkyl linker, or is a region of nucleotides comprising an inverted nucleoside linkage.

73. The 5′ capture probe according to claim 71 wherein the linker region III comprises or is a discontinuity resulting in a two stranded 5′ capture probe, the first strand comprising the first and second regions, and a second strand comprising the fourth and fifth regions.

74. The 5′ capture probe according to claim 65, wherein region V comprises a predetermined region of nucleotides which are complementary to the 5′ region of a modified oligonucleotide.

75. The 5′ capture probe according to claim 65, wherein region V comprises a degenerate sequence of nucleotides.

76. The 5′ capture probe according to claim 65, wherein the 5′ terminus of region V comprises a fluorescent label such as 5′-FAM.

77. A kit for use in the capture of modified oligonucleotides, said kit comprising a 3′ capture probe and a 5′ capture probe, wherein the 5′ capture probe is as according to claim 65.
